SEQUENCE LISTING (4018 sequences)

SEQ ID NO: 1 - protein, 9 aa, synthetic construct
  REGION 1..9: note = Synthetic: 2 - VH CDR1
FTFSEYTMN

SEQ ID NO: 2 - protein, 17 aa, synthetic construct
  REGION 1..17: note = Synthetic: 2 - VH CDR2
AIVGSGDSTYYADSVKG

SEQ ID NO: 3 - protein, 16 aa, synthetic construct
  REGION 1..16: note = Synthetic: 2 - VH CDR3
AKDYSSGDWIDYGMDV

SEQ ID NO: 4 - protein, 11 aa, synthetic construct
  REGION 1..11: note = Synthetic: 2 - VL CDR1
QASQDISNYLN

SEQ ID NO: 5 - protein, 7 aa, synthetic construct
  REGION 1..7: note = Synthetic: 2 - VL CDR2
DASNLAT

SEQ ID NO: 6 - protein, 9 aa, synthetic construct
  REGION 1..9: note = Synthetic: 2 - VL CDR3
QQFDLLPPT

SEQ ID NO: 7 - protein, 26 aa, synthetic construct
  REGION 1..26: note = Synthetic: 2 - VH FR1
EVQLVESGGGLVKPGGSLRLSCAASG

SEQ ID NO: 8 - protein, 14 aa, synthetic construct
  REGION 1..14: note = Synthetic: 2 - VH FR2
WVRQAPGKGLEWVS

SEQ ID NO: 9 - protein, 30 aa, synthetic construct
  REGION 1..30: note = Synthetic: 2 - VH FR3
RFTISRDNSKNTLYLQMNSLRAEDTAVYYC

SEQ ID NO: 10 - protein, 11 aa, synthetic construct
  REGION 1..11: note = Synthetic: 2 - VH FR4
WGQGTTVTVSS

SEQ ID NO: 11 - protein, 369 aa, synthetic construct
  REGION 1..369: note = Synthetic: 2 - VH DNA
GAGGTGCAGCTGGTGGAGTCTGGGGGAGGCCTGGTCAAGCCTGGGGGGTCCCTGAGACTCTCCTGTGCAGCCTCTGGATTCACCTTCAGTGAATATACCATGAACTGGGTCCGCCAGGCTCCAGGGAAGGGGCTGGAGTGGGTCTCAGCTATTGTAGGTAGTGGTGACAGCACATACTACGCAGACTCCGTGAAGGGCCGGTTCACCATCTCCAGAGACAATTCCAAGAACACGCTGTATCTGCAAATGAACAGCCTGAGAGCCGAGGACACGGCGGTGTACTACTGCGCCAAGGACTACAGCTCCGGAGACTGGATCGATTATGGAATGGACGTATGGGGCCAGGGAACAACTGTCACCGTCTCCTCA

SEQ ID NO: 12 - protein, 123 aa, synthetic construct
  REGION 1..123: note = Synthetic: 2 - VH Protein
EVQLVESGGGLVKPGGSLRLSCAASGFTFSEYTMNWVRQAPGKGLEWVSAIVGSGDSTYYADSVKGRFTISRDNSKNTLYLQMNSLRAEDTAVYYCAKDYSSGDWIDYGMDVWGQGTTVTVSS

SEQ ID NO: 13 - protein, 23 aa, synthetic construct
  REGION 1..23: note = Synthetic: 2 - VL FR1
DIQMTQSPSSLSASVGDRVTITC

SEQ ID NO: 14 - protein, 15 aa, synthetic construct
  REGION 1..15: note = Synthetic: 2 - VL FR2
WYQQKPGKAPKLLIY

SEQ ID NO: 15 - protein, 32 aa, synthetic construct
  REGION 1..32: note = Synthetic: 2 - VL FR3
GVPSRFSGSGSGTDFTFTISSLQPEDIATYYC

SEQ ID NO: 16 - protein, 10 aa, synthetic construct
  REGION 1..10: note = Synthetic: 2 - VL FR4
FGGGTKVEIK

SEQ ID NO: 17 - protein, 321 aa, synthetic construct
  REGION 1..321: note = Synthetic: 2 - VL DNA
GACATCCAGATGACCCAGTCTCCATCCTCCCTGTCTGCATCTGTAGGAGACAGAGTCACCATCACTTGCCAGGCGAGTCAGGACATTAGCAACTATTTAAATTGGTATCAGCAGAAACCAGGGAAAGCCCCTAAGCTCCTGATCTACGATGCATCCAATTTGGCAACAGGGGTCCCATCAAGGTTCAGTGGAAGTGGATCTGGGACAGATTTTACTTTCACCATCAGCAGCCTGCAGCCTGAAGATATTGCAACATATTACTGTCAGCAGTTCGATCTCCTCCCTCCTACTTTTGGCGGAGGGACCAAGGTTGAGATCAAA

SEQ ID NO: 18 - protein, 107 aa, synthetic construct
  REGION 1..107: note = Synthetic: 2 - VL Protein
DIQMTQSPSSLSASVGDRVTITCQASQDISNYLNWYQQKPGKAPKLLIYDASNLATGVPSRFSGSGSGTDFTFTISSLQPEDIATYYCQQFDLLPPTFGGGTKVEIK

SEQ ID NO: 19-100 - skipped (82 entries)

SEQ ID NO: 101 - protein, 9 aa, synthetic construct
  REGION 1..9: note = Synthetic: 5 - VH CDR1
FTFSDYAMI

SEQ ID NO: 102 - protein, 17 aa, synthetic construct
  REGION 1..17: note = Synthetic: 5 - VH CDR2
AISGGGESTYYADSVKG

SEQ ID NO: 103 - protein, 16 aa, synthetic construct
  REGION 1..16: note = Synthetic: 5 - VH CDR3
AKDYSSGDWIDYGMDV

SEQ ID NO: 104 - protein, 11 aa, synthetic construct
  REGION 1..11: note = Synthetic: 5 - VL CDR1
QASQDISNYLN

SEQ ID NO: 105 - protein, 7 aa, synthetic construct
  REGION 1..7: note = Synthetic: 5 - VL CDR2
DASNLAT

SEQ ID NO: 106 - protein, 9 aa, synthetic construct
  REGION 1..9: note = Synthetic: 5 - VL CDR3
QQFDLLPPT

SEQ ID NO: 107 - protein, 26 aa, synthetic construct
  REGION 1..26: note = Synthetic: 5 - VH FR1
EVQLLESGGGLVQPGGSLRLSCAASG

SEQ ID NO: 108 - protein, 14 aa, synthetic construct
  REGION 1..14: note = Synthetic: 5 - VH FR2
WVRQAPGKGLEWVS

SEQ ID NO: 109 - protein, 30 aa, synthetic construct; identical to SEQ ID NO: 9
  REGION 1..30: note = Synthetic: 5 - VH FR3

SEQ ID NO: 110 - protein, 11 aa, synthetic construct
  REGION 1..11: note = Synthetic: 5 - VH FR4
WGQGTTVTVSS

SEQ ID NO: 111 - DNA, 369 bp, synthetic construct
  misc_feature 1..369: note = Synthetic: 5 - VH DNA
  source 1..369: mol_type = other DNA
gaggtgcagctgttggagtctgggggaggcttggtacagcctggggggtccctgagactctcctgtgcagcctctggattcacctttagcgactatgccatgatatgggtccgccaggctccagggaaggggctggagtgggtctcagctattagtggtggaggtgaaagcacatactacgcagactccgtgaagggccggttcaccatctccagagacaattccaagaacacgctgtatctgcaaatgaacagcctgagagccgaggacacggcggtgtactactgcgccaaggactacagctccggagactggatcgattatggaatggacgtatggggccagggaacaactgtcaccgtctcctca

SEQ ID NO: 112 - protein, 123 aa, synthetic construct
  REGION 1..123: note = Synthetic: 5 - VH Protein
EVQLLESGGGLVQPGGSLRLSCAASGFTFSDYAMIWVRQAPGKGLEWVSAISGGGESTYYADSVKGRFTISRDNSKNTLYLQMNSLRAEDTAVYYCAKDYSSGDWIDYGMDVWGQGTTVTVSS

SEQ ID NO: 113 - protein, 23 aa, synthetic construct
  REGION 1..23: note = Synthetic: 5 - VL FR1
DIQMTQSPSSLSASVGDRVTITC

SEQ ID NO: 114 - protein, 15 aa, synthetic construct
  REGION 1..15: note = Synthetic: 5 - VL FR2
WYQQKPGKAPKLLIY

SEQ ID NO: 115 - protein, 32 aa, synthetic construct; identical to SEQ ID NO: 15
  REGION 1..32: note = Synthetic: 5 - VL FR3

SEQ ID NO: 116 - protein, 10 aa, synthetic construct
  REGION 1..10: note = Synthetic: 5 - VL FR4
FGGGTKVEIK

SEQ ID NO: 117 - DNA, 321 bp, synthetic construct
  misc_feature 1..321: note = Synthetic: 5 - VL DNA
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggcaacaggggtcccatcaaggttcagtggaagtggatctgggacagattttactttcaccatcagcagcctgcagcctgaagatattgcaacatattactgtcagcagttcgatctcctccctcctacttttggcggagggaccaaggttgagatcaaa

SEQ ID NO: 118 - protein, 107 aa, synthetic construct; identical to SEQ ID NO: 18
  REGION 1..107: note = Synthetic: 5 - VL Protein

SEQ ID NO: 119-200 - skipped (82 entries)

SEQ ID NO: 201 - protein, 9 aa, synthetic construct
  REGION 1..9: note = Synthetic: 44 - VH CDR1
GTFDNYYIS

SEQ ID NO: 202 - protein, 17 aa, synthetic construct
  REGION 1..17: note = Synthetic: 44 - VH CDR2
GIFPIFGTANYAQKFQG

SEQ ID NO: 203 - protein, 16 aa, synthetic construct
  REGION 1..16: note = Synthetic: 44 - VH CDR3
AREVGHYSGSPYYMDV

SEQ ID NO: 204 - protein, 11 aa, synthetic construct
  REGION 1..11: note = Synthetic: 44 - VL CDR1
RASQSINSWLA

SEQ ID NO: 205 - protein, 7 aa, synthetic construct
  REGION 1..7: note = Synthetic: 44 - VL CDR2
DASSLES

SEQ ID NO: 206 - protein, 8 aa, synthetic construct
  REGION 1..8: note = Synthetic: 44 - VL CDR3
QQVGPYLT

SEQ ID NO: 207 - protein, 26 aa, synthetic construct
  REGION 1..26: note = Synthetic: 44 - VH FR1
QVQLVQSGAEVKKPGSSVKVSCKASG

SEQ ID NO: 208 - protein, 14 aa, synthetic construct
  REGION 1..14: note = Synthetic: 44 - VH FR2
WVRQAPGQGLEWMG

SEQ ID NO: 209 - protein, 30 aa, synthetic construct
  REGION 1..30: note = Synthetic: 44 - VH FR3
RVTITADESTSTAYMELSSLRSEDTAVYYC

SEQ ID NO: 210 - protein, 11 aa, synthetic construct
  REGION 1..11: note = Synthetic: 44 - VH FR4
WGKGTTVTVSS

SEQ ID NO: 211 - DNA, 369 bp, synthetic construct
  misc_feature 1..369: note = Synthetic: 44 - VH DNA
  source 1..369: mol_type = other DNA
caggtgcagctggtgcagtctggggctgaggtgaagaagcctgggtcctcggtgaaggtctcctgcaaggcttctggaggcaccttcgacaactattacatcagctgggtgcgacaggcccctggacaagggcttgagtggatgggagggatcttccctatcttcggtaccgcaaactacgcacagaagttccagggcagagtcacgattaccgcggacgaatccacgagcacagcctacatggagctgagcagcctgagatctgaggacacggcggtgtactactgcgccagagaagtcggacactactccggcagcccatactacatggacgtatggggcaagggtacaactgtcaccgtctcctca

SEQ ID NO: 212 - protein, 123 aa, synthetic construct
  REGION 1..123: note = Synthetic: 44 - VH Protein
QVQLVQSGAEVKKPGSSVKVSCKASGGTFDNYYISWVRQAPGQGLEWMGGIFPIFGTANYAQKFQGRVTITADESTSTAYMELSSLRSEDTAVYYCAREVGHYSGSPYYMDVWGKGTTVTVSS

SEQ ID NO: 213 - protein, 23 aa, synthetic construct
  REGION 1..23: note = Synthetic: 44 - VL FR1
DIQMTQSPSTLSASVGDRVTITC

SEQ ID NO: 214 - protein, 15 aa, synthetic construct
  REGION 1..15: note = Synthetic: 44 - VL FR2
WYQQKPGKAPKLLIS

SEQ ID NO: 215 - protein, 32 aa, synthetic construct
  REGION 1..32: note = Synthetic: 44 - VL FR3
GVPSRFSGSGSGTEFTLTISSLQPDDFATYYC

SEQ ID NO: 216 - protein, 10 aa, synthetic construct
  REGION 1..10: note = Synthetic: 44 - VL FR4
FGGGTKVEIK

SEQ ID NO: 217 - DNA, 318 bp, synthetic construct
  misc_feature 1..318: note = Synthetic: 44 - VL DNA
  source 1..318: mol_type = other DNA
gacatccagatgacccagtctccttccaccctgtctgcatctgtaggagacagagtcaccatcacttgccgggccagtcagagtattaatagctggttggcctggtatcagcagaaaccagggaaagcccctaagctcctgatctccgatgcctccagtttggaaagtggggtcccatcaaggttcagcggcagtggatctgggacagaattcactctcaccatcagcagcctgcagcctgatgattttgcaacttattactgccagcaggtcggcccctacctcacttttggcggagggaccaaggttgagatcaaa

SEQ ID NO: 218 - protein, 106 aa, synthetic construct
  REGION 1..106: note = Synthetic: 44 - VL Protein
DIQMTQSPSTLSASVGDRVTITCRASQSINSWLAWYQQKPGKAPKLLISDASSLESGVPSRFSGSGSGTEFTLTISSLQPDDFATYYCQQVGPYLTFGGGTKVEIK

SEQ ID NO: 219-300 - skipped (82 entries)

SEQ ID NO: 301 - protein, 9 aa, synthetic construct
  REGION 1..9: note = Synthetic: 58 - VH CDR1
FTFGDYAMS

SEQ ID NO: 302 - protein, 19 aa, synthetic construct
  REGION 1..19: note = Synthetic: 58 - VH CDR2
FIGSKFYGGETEYTASVKG

SEQ ID NO: 303 - protein, 13 aa, synthetic construct
  REGION 1..13: note = Synthetic: 58 - VH CDR3
ARGPRRYTYGMDV

SEQ ID NO: 304 - protein, 11 aa, synthetic construct
  REGION 1..11: note = Synthetic: 58 - VL CDR1
RASQSISSYLN

SEQ ID NO: 305 - protein, 7 aa, synthetic construct
  REGION 1..7: note = Synthetic: 58 - VL CDR2
AASSLQS

SEQ ID NO: 306 - protein, 8 aa, synthetic construct
  REGION 1..8: note = Synthetic: 58 - VL CDR3
QQSSTPLT

SEQ ID NO: 307 - protein, 26 aa, synthetic construct
  REGION 1..26: note = Synthetic: 58 - VH FR1
EVQLVESGGGLVQPGRSLRLSCTASG

SEQ ID NO: 308 - protein, 14 aa, synthetic construct
  REGION 1..14: note = Synthetic: 58 - VH FR2
WFRQAPGKGLEWVG

SEQ ID NO: 309 - protein, 30 aa, synthetic construct
  REGION 1..30: note = Synthetic: 58 - VH FR3
RFTISRDGSKSIAYLQMNSLKTEDTAVYYC

SEQ ID NO: 310 - protein, 11 aa, synthetic construct
  REGION 1..11: note = Synthetic: 58 - VH FR4
WGQGTTVTVSS

SEQ ID NO: 311 - DNA, 366 bp, synthetic construct
  misc_feature 1..366: note = Synthetic: 58 - VH DNA
  source 1..366: mol_type = other DNA
gaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattggaagcaaattctatggtggggaaacagaatacaccgcgtctgtgaaaggcagattcaccatctcaagagatggttccaaaagcatcgcctatctgcaaatgaacagcctgaaaaccgaggacacggcggtgtactactgcgccagaggaccaagacgctacacatacggaatggacgtatggggccagggaacaactgtcaccgtctcctca

SEQ ID NO: 312 - protein, 122 aa, synthetic construct
  REGION 1..122: note = Synthetic: 58 - VH Protein
EVQLVESGGGLVQPGRSLRLSCTASGFTFGDYAMSWFRQAPGKGLEWVGFIGSKFYGGETEYTASVKGRFTISRDGSKSIAYLQMNSLKTEDTAVYYCARGPRRYTYGMDVWGQGTTVTVSS

SEQ ID NO: 313 - protein, 23 aa, synthetic construct
  REGION 1..23: note = Synthetic: 58 - VL FR1
DIQMTQSPSSLSASVGDRVTITC

SEQ ID NO: 314 - protein, 15 aa, synthetic construct
  REGION 1..15: note = Synthetic: 58 - VL FR2
WYQQKPGKAPKLLIY

SEQ ID NO: 315 - protein, 32 aa, synthetic construct
  REGION 1..32: note = Synthetic: 58 - VL FR3
GVPSRFSGSGSGTDFTLTISSLQPEDFATYYC

SEQ ID NO: 316 - protein, 10 aa, synthetic construct
  REGION 1..10: note = Synthetic: 58 - VL FR4
FGGGTKVEIK

SEQ ID NO: 317 - DNA, 318 bp, synthetic construct
  misc_feature 1..318: note = Synthetic: 58 - VL DNA
  source 1..318: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagctccacccccctcacttttggcggagggaccaaggttgagatcaaa

SEQ ID NO: 318 - protein, 106 aa, synthetic construct
  REGION 1..106: note = Synthetic: 58 - VL Protein
DIQMTQSPSSLSASVGDRVTITCRASQSISSYLNWYQQKPGKAPKLLIYAASSLQSGVPSRFSGSGSGTDFTLTISSLQPEDFATYYCQQSSTPLTFGGGTKVEIK

SEQ ID NO: 319-400 - skipped (82 entries)

SEQ ID NO: 401 - protein, 9 aa, synthetic construct
  REGION 1..9: note = Synthetic: 10 - VH CDR1
FTFDDYAVH

SEQ ID NO: 402 - protein, 17 aa, synthetic construct
  REGION 1..17: note = Synthetic: 10 - VH CDR2
GISWSSGLIGYADSVKG

SEQ ID NO: 403 - protein, 13 aa, synthetic construct
  REGION 1..13: note = Synthetic: 10 - VH CDR3
AKGPPTYQDYFDL

SEQ ID NO: 404 - protein, 11 aa, synthetic construct
  REGION 1..11: note = Synthetic: 10 - VL CDR1
RASQSVSRYLA

SEQ ID NO: 405 - protein, 7 aa, synthetic construct
  REGION 1..7: note = Synthetic: 10 - VL CDR2
DASNRAT

SEQ ID NO: 406 - protein, 10 aa, synthetic construct
  REGION 1..10: note = Synthetic: 10 - VL CDR3
QQVSFFPPIT

SEQ ID NO: 407 - protein, 26 aa, synthetic construct
  REGION 1..26: note = Synthetic: 10 - VH FR1
EVQLVESGGGLVQPGRSLRLSCAASG

SEQ ID NO: 408 - protein, 14 aa, synthetic construct
  REGION 1..14: note = Synthetic: 10 - VH FR2
WVRQAPGKGLEWVS

SEQ ID NO: 409 - protein, 30 aa, synthetic construct
  REGION 1..30: note = Synthetic: 10 - VH FR3
RFTISRDNAKNSLYLQMNSLRAEDTAVYYC

SEQ ID NO: 410 - protein, 11 aa, synthetic construct
  REGION 1..11: note = Synthetic: 10 - VH FR4
WGRGTLVTVSS

SEQ ID NO: 411 - DNA, 360 bp, synthetic construct
  misc_feature 1..360: note = Synthetic: 10 - VH DNA
  source 1..360: mol_type = other DNA
gaagtgcagctggtggagtctgggggaggcttggtacagcctggcaggtccctgagactctcctgtgcagcctctggattcacctttgatgattatgccgtgcactgggtccggcaagctccagggaagggcctggagtgggtctcaggtattagttggagtagtggactaataggctatgcggactctgtgaagggccgattcaccatctccagagacaacgccaagaactccctgtatctgcaaatgaacagtctgagagctgaggacacggcggtgtactactgcgccaagggccctcctacctaccaagactacttcgacctatgggggagaggtaccttggtcaccgtctcctca

SEQ ID NO: 412 - protein, 120 aa, synthetic construct
  REGION 1..120: note = Synthetic: 10 - VH Protein
EVQLVESGGGLVQPGRSLRLSCAASGFTFDDYAVHWVRQAPGKGLEWVSGISWSSGLIGYADSVKGRFTISRDNAKNSLYLQMNSLRAEDTAVYYCAKGPPTYQDYFDLWGRGTLVTVSS

SEQ ID NO: 413 - protein, 23 aa, synthetic construct
  REGION 1..23: note = Synthetic: 10 - VL FR1
EIVLTQSPATLSLSPGERATLSC

SEQ ID NO: 414 - protein, 15 aa, synthetic construct
  REGION 1..15: note = Synthetic: 10 - VL FR2
WYQQKPGQAPRLLIY

SEQ ID NO: 415 - protein, 32 aa, synthetic construct
  REGION 1..32: note = Synthetic: 10 - VL FR3
GIPARFSGSGSGTDFTLTISSLEPEDFAVYYC

SEQ ID NO: 416 - protein, 10 aa, synthetic construct
  REGION 1..10: note = Synthetic: 10 - VL FR4
FGGGTKVEIK

SEQ ID NO: 417 - DNA, 324 bp, synthetic construct
  misc_feature 1..324: note = Synthetic: 10 - VL DNA
  source 1..324: mol_type = other DNA
gaaattgtgttgacacagtctccagccaccctgtctttgtctccaggggaaagagccaccctctcctgcagggccagtcagagtgttagcaggtacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaggtcagtttcttccctcctatcacttttggcggagggaccaaggttgagatcaaa

SEQ ID NO: 418 - protein, 108 aa, synthetic construct
  REGION 1..108: note = Synthetic: 10 - VL Protein
EIVLTQSPATLSLSPGERATLSCRASQSVSRYLAWYQQKPGQAPRLLIYDASNRATGIPARFSGSGSGTDFTLTISSLEPEDFAVYYCQQVSFFPPITFGGGTKVEIK

SEQ ID NO: 419-500 - skipped (82 entries)

SEQ ID NO: 501 - protein, 9 aa, synthetic construct
  REGION 1..9: note = Synthetic: 38 - VH CDR1
FTFSGHLMS

SEQ ID NO: 502 - protein, 17 aa, synthetic construct
  REGION 1..17: note = Synthetic: 38 - VH CDR2
AISGSAGETYYADSVKG

SEQ ID NO: 503 - protein, 19 aa, synthetic construct
  REGION 1..19: note = Synthetic: 38 - VH CDR3
ARDAYYDDWSGWADWYFDL

SEQ ID NO: 504 - protein, 11 aa, synthetic construct
  REGION 1..11: note = Synthetic: 38 - VL CDR1
RASQSVSRYLA

SEQ ID NO: 505 - protein, 7 aa, synthetic construct
  REGION 1..7: note = Synthetic: 38 - VL CDR2
DASNRAT

SEQ ID NO: 506 - protein, 9 aa, synthetic construct
  REGION 1..9: note = Synthetic: 38 - VL CDR3
QQVSLLPPT

SEQ ID NO: 507 - protein, 26 aa, synthetic construct
  REGION 1..26: note = Synthetic: 38 - VH FR1
EVQLLESGGGLVQPGGSLRLSCAASG

SEQ ID NO: 508 - protein, 14 aa, synthetic construct
  REGION 1..14: note = Synthetic: 38 - VH FR2
WVRQAPGKGLEWVS

SEQ ID NO: 509 - protein, 30 aa, synthetic construct; identical to SEQ ID NO: 9
  REGION 1..30: note = Synthetic: 38 - VH FR3

SEQ ID NO: 510 - protein, 11 aa, synthetic construct
  REGION 1..11: note = Synthetic: 38 - VH FR4
WGRGTLVTVSS

SEQ ID NO: 511 - DNA, 378 bp, synthetic construct
  misc_feature 1..378: note = Synthetic: 38 - VH DNA
  source 1..378: mol_type = other DNA
gaggtgcagctgttggagtctgggggaggcttggtacagcctggggggtccctgagactctcctgtgcagcctctggattcacctttagcggacacctaatgagctgggtccgccaggctccagggaaggggctggagtgggtctcagctattagtggatccgcaggtgaaacatactacgcagactccgtgaagggccggttcaccatctccagagacaattccaagaacacgctgtatctgcaaatgaacagcctgagagccgaggacacggcggtgtactactgcgccagagatgcgtactacgacgactggagcggatgggccgattggtacttcgatttatgggggagaggtaccttggtcaccgtctcctca

SEQ ID NO: 512 - protein, 126 aa, synthetic construct
  REGION 1..126: note = Synthetic: 38 - VH Protein
EVQLLESGGGLVQPGGSLRLSCAASGFTFSGHLMSWVRQAPGKGLEWVSAISGSAGETYYADSVKGRFTISRDNSKNTLYLQMNSLRAEDTAVYYCARDAYYDDWSGWADWYFDLWGRGTLVTVSS

SEQ ID NO: 513 - protein, 23 aa, synthetic construct
  REGION 1..23: note = Synthetic: 38 - VL FR1
EIVLTQSPATLSLSPGERATLSC

SEQ ID NO: 514 - protein, 15 aa, synthetic construct
  REGION 1..15: note = Synthetic: 38 - VL FR2
WYQQKPGQAPRLLIY

SEQ ID NO: 515 - protein, 32 aa, synthetic construct; identical to SEQ ID NO: 415
  REGION 1..32: note = Synthetic: 38 - VL FR3

SEQ ID NO: 516 - protein, 10 aa, synthetic construct
  REGION 1..10: note = Synthetic: 38 - VL FR4
FGGGTKVEIK

SEQ ID NO: 517 - DNA, 321 bp, synthetic construct
  misc_feature 1..321: note = Synthetic: 38 - VL DNA
  source 1..321: mol_type = other DNA
gaaattgtgttgacacagtctccagccaccctgtctttgtctccaggggaaagagccaccctctcctgcagggccagtcagagtgttagcaggtacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcaggtcagtctcctccctcctacttttggcggagggaccaaggttgagatcaaa

SEQ ID NO: 518 - protein, 107 aa, synthetic construct
  REGION 1..107: note = Synthetic: 38 - VL Protein
EIVLTQSPATLSLSPGERATLSCRASQSVSRYLAWYQQKPGQAPRLLIYDASNRATGIPARFSGSGSGTDFTLTISSLEPEDFAVYYCQQVSLLPPTFGGGTKVEIK

SEQ ID NO: 519-600 - skipped (82 entries)

SEQ ID NO: 601 - protein, 9 aa, synthetic construct
  REGION 1..9: note = Synthetic: 15 - VH CDR1
FTFGDVAMS

SEQ ID NO: 602 - protein, 19 aa, synthetic construct
  REGION 1..19: note = Synthetic: 15 - VH CDR2
YIGSKAYGGETEYTASVKG

SEQ ID NO: 603 - protein, 17 aa, synthetic construct
  REGION 1..17: note = Synthetic: 15 - VH CDR3
ARAGHSYGSIASNWFDP

SEQ ID NO: 604 - protein, 11 aa, synthetic construct
  REGION 1..11: note = Synthetic: 15 - VL CDR1
RASQSISSYLN

SEQ ID NO: 605 - protein, 7 aa, synthetic construct
  REGION 1..7: note = Synthetic: 15 - VL CDR2
GASSLQS

SEQ ID NO: 606 - protein, 9 aa, synthetic construct
  REGION 1..9: note = Synthetic: 15 - VL CDR3
QQGFYTPWT

SEQ ID NO: 607 - protein, 26 aa, synthetic construct
  REGION 1..26: note = Synthetic: 15 - VH FR1
EVQLVESGGGLVQPGRSLRLSCTASG

SEQ ID NO: 608 - protein, 14 aa, synthetic construct
  REGION 1..14: note = Synthetic: 15 - VH FR2
WFRQAPGKGLEWVG

SEQ ID NO: 609 - protein, 30 aa, synthetic construct; identical to SEQ ID NO: 309
  REGION 1..30: note = Synthetic: 15 - VH FR3

SEQ ID NO: 610 - protein, 11 aa, synthetic construct
  REGION 1..11: note = Synthetic: 15 - VH FR4
WGQGTLVTVSS

SEQ ID NO: 611 - DNA, 378 bp, synthetic construct
  misc_feature 1..378: note = Synthetic: 15 - VH DNA
  source 1..378: mol_type = other DNA
gaggtgcagctggtggagtctgggggaggcttggtacagccagggcggtccctgagactctcctgtacagcttctggattcacctttggtgatgtcgctatgtcctggttccgccaggctccagggaaggggctggagtgggtaggttacattggaagcaaagcttatggtggggaaacagaatacaccgcgtctgtgaaaggcagattcaccatctcaagagatggttccaaaagcatcgcctatctgcaaatgaacagcctgaaaaccgaggacacggcggtgtactactgcgccagagctggacacagctacggatccatcgccagcaactggttcgacccatggggacagggtacattggtcaccgtctcctca

SEQ ID NO: 612 - protein, 126 aa, synthetic construct
  REGION 1..126: note = Synthetic: 15 - VH Protein
EVQLVESGGGLVQPGRSLRLSCTASGFTFGDVAMSWFRQAPGKGLEWVGYIGSKAYGGETEYTASVKGRFTISRDGSKSIAYLQMNSLKTEDTAVYYCARAGHSYGSIASNWFDPWGQGTLVTVSS

SEQ ID NO: 613 - protein, 23 aa, synthetic construct
  REGION 1..23: note = Synthetic: 15 - VL FR1
DIQMTQSPSSLSASVGDRVTITC

SEQ ID NO: 614 - protein, 15 aa, synthetic construct
  REGION 1..15: note = Synthetic: 15 - VL FR2
WYQQKPGKAPKLLIY

SEQ ID NO: 615 - protein, 32 aa, synthetic construct; identical to SEQ ID NO: 315
  REGION 1..32: note = Synthetic: 15 - VL FR3

SEQ ID NO: 616 - protein, 10 aa, synthetic construct
  REGION 1..10: note = Synthetic: 15 - VL FR4
FGGGTKVEIK

SEQ ID NO: 617 - DNA, 321 bp, synthetic construct
  misc_feature 1..321: note = Synthetic: 15 - VL DNA
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaaggattctacactccttggacttttggcggagggaccaaggttgagatcaaa

SEQ ID NO: 618 - protein, 107 aa, synthetic construct
  REGION 1..107: note = Synthetic: 15 - VL Protein
DIQMTQSPSSLSASVGDRVTITCRASQSISSYLNWYQQKPGKAPKLLIYGASSLQSGVPSRFSGSGSGTDFTLTISSLQPEDFATYYCQQGFYTPWTFGGGTKVEIK

SEQ ID NO: 619-700 - skipped (82 entries)

SEQ ID NO: 701 - protein, 9 aa, synthetic construct
  REGION 1..9: note = Synthetic: 35 - VH CDR1
GTFSSAAIS

SEQ ID NO: 702 - protein, 17 aa, synthetic construct
  REGION 1..17: note = Synthetic: 35 - VH CDR2
NIIPIVGIANYAQKFQG

SEQ ID NO: 703 - protein, 16 aa, synthetic construct
  REGION 1..16: note = Synthetic: 35 - VH CDR3
ARDTGRGYTRHFWFDP

SEQ ID NO: 704 - protein, 11 aa, synthetic construct
  REGION 1..11: note = Synthetic: 35 - VL CDR1
RASQSISSYLN

SEQ ID NO: 705 - protein, 7 aa, synthetic construct
  REGION 1..7: note = Synthetic: 35 - VL CDR2
AASSLQS

SEQ ID NO: 706 - protein, 8 aa, synthetic construct
  REGION 1..8: note = Synthetic: 35 - VL CDR3
QQSDILYT

SEQ ID NO: 707 - protein, 26 aa, synthetic construct
  REGION 1..26: note = Synthetic: 35 - VH FR1
QVQLVQSGAEVKKPGSSVKVSCKASG

SEQ ID NO: 708 - protein, 14 aa, synthetic construct
  REGION 1..14: note = Synthetic: 35 - VH FR2
WVRQAPGQGLEWMG

SEQ ID NO: 709 - protein, 30 aa, synthetic construct; identical to SEQ ID NO: 209
  REGION 1..30: note = Synthetic: 35 - VH FR3

SEQ ID NO: 710 - protein, 11 aa, synthetic construct
  REGION 1..11: note = Synthetic: 35 - VH FR4
WGQGTLVTVSS

SEQ ID NO: 711 - DNA, 369 bp, synthetic construct
  misc_feature 1..369: note = Synthetic: 35 - VH DNA
  source 1..369: mol_type = other DNA
caggtgcagctggtgcagtctggggctgaggtgaagaagcctgggtcctcggtgaaggtctcctgcaaggcttctggaggcaccttcagctccgccgctatcagctgggtgcgacaggcccctggacaagggcttgagtggatgggaaacatcatccctatcgtaggtatagcaaactacgcacagaagttccagggcagagtcacgattaccgcggacgaatccacgagcacagcctacatggagctgagcagcctgagatctgaggacacggcggtgtactactgcgccagagacacgggacggggatacaccagacacttctggtttgacccctggggacagggtacattggtcaccgtctcctca

SEQ ID NO: 712 - protein, 123 aa, synthetic construct
  REGION 1..123: note = Synthetic: 35 - VH Protein
QVQLVQSGAEVKKPGSSVKVSCKASGGTFSSAAISWVRQAPGQGLEWMGNIIPIVGIANYAQKFQGRVTITADESTSTAYMELSSLRSEDTAVYYCARDTGRGYTRHFWFDPWGQGTLVTVSS

SEQ ID NO: 713 - protein, 23 aa, synthetic construct
  REGION 1..23: note = Synthetic: 35 - VL FR1
DIQMTQSPSSLSASVGDRVTITC

SEQ ID NO: 714 - protein, 15 aa, synthetic construct
  REGION 1..15: note = Synthetic: 35 - VL FR2
WYQQKPGKAPKLLIY

SEQ ID NO: 715 - protein, 32 aa, synthetic construct; identical to SEQ ID NO: 315
  REGION 1..32: note = Synthetic: 35 - VL FR3

SEQ ID NO: 716 - protein, 10 aa, synthetic construct
  REGION 1..10: note = Synthetic: 35 - VL FR4
FGGGTKVEIK

SEQ ID NO: 717 - DNA, 318 bp, synthetic construct
  misc_feature 1..318: note = Synthetic: 35 - VL DNA
  source 1..318: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagcgacatcctctacacttttggcggagggaccaaggttgagatcaaa

SEQ ID NO: 718 - protein, 106 aa, synthetic construct
  REGION 1..106: note = Synthetic: 35 - VL Protein
DIQMTQSPSSLSASVGDRVTITCRASQSISSYLNWYQQKPGKAPKLLIYAASSLQSGVPSRFSGSGSGTDFTLTISSLQPEDFATYYCQQSDILYTFGGGTKVEIK

SEQ ID NO: 719-800 - skipped (82 entries)

SEQ ID NO: 801 - protein, 9 aa, synthetic construct
  REGION 1..9: note = Synthetic: 47 - VH CDR1
GTFSNYAIS

SEQ ID NO: 802 - protein, 17 aa, synthetic construct
  REGION 1..17: note = Synthetic: 47 - VH CDR2
GIIPIFGTANYAQKFQG

SEQ ID NO: 803 - protein, 18 aa, synthetic construct
  REGION 1..18: note = Synthetic: 47 - VH CDR3
ARGRGALALVGPYYGMDV

SEQ ID NO: 804 - protein, 16 aa, synthetic construct
  REGION 1..16: note = Synthetic: 47 - VL CDR1
RSSQSLLHSNGYNYLD

SEQ ID NO: 805 - protein, 7 aa, synthetic construct
  REGION 1..7: note = Synthetic: 47 - VL CDR2
LGSHRAS

SEQ ID NO: 806 - protein, 8 aa, synthetic construct
  REGION 1..8: note = Synthetic: 47 - VL CDR3
MQALRAPT

SEQ ID NO: 807 - protein, 26 aa, synthetic construct
  REGION 1..26: note = Synthetic: 47 - VH FR1
EVQLVQSGAEVKKPGSSVKVSCKASG

SEQ ID NO: 808 - protein, 14 aa, synthetic construct
  REGION 1..14: note = Synthetic: 47 - VH FR2
WVRQAPGQGLEWMG

SEQ ID NO: 809 - protein, 30 aa, synthetic construct; identical to SEQ ID NO: 209
  REGION 1..30: note = Synthetic: 47 - VH FR3

SEQ ID NO: 810 - protein, 11 aa, synthetic construct
  REGION 1..11: note = Synthetic: 47 - VH FR4
WGQGTTVTVSS

SEQ ID NO: 811 - DNA, 375 bp, synthetic construct
  misc_feature 1..375: note = Synthetic: 47 - VH DNA
  source 1..375: mol_type = other DNA
gaggtgcagctggtgcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagaggcagaggcgctctggcactcgtcggaccatactacggaatggacgtatggggccagggaacaactgtcaccgtctcctca

SEQ ID NO: 812 - protein, 125 aa, synthetic construct
  REGION 1..125: note = Synthetic: 47 - VH Protein
EVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGRGALALVGPYYGMDVWGQGTTVTVSS

SEQ ID NO: 813 - protein, 23 aa, synthetic construct
  REGION 1..23: note = Synthetic: 47 - VL FR1
DIVMTQSPLSLPVTPGEPASISC

SEQ ID NO: 814 - protein, 15 aa, synthetic construct
  REGION 1..15: note = Synthetic: 47 - VL FR2
WYLQKPGQSPQLLIY

SEQ ID NO: 815 - protein, 32 aa, synthetic construct
  REGION 1..32: note = Synthetic: 47 - VL FR3
GVPDRFSGSGSGTDFTLKISRVEAEDVGVYYC

SEQ ID NO: 816 - protein, 10 aa, synthetic construct
  REGION 1..10: note = Synthetic: 47 - VL FR4
FGGGTKVEIK

SEQ ID NO: 817 - DNA, 333 bp, synthetic construct
  misc_feature 1..333: note = Synthetic: 47 - VL DNA
  source 1..333: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctcatcgggcctccggggtccctgacaggttcagtggcagtggatcaggcacagattttacactgaaaatcagcagagtggaggctgaggatgttggggtttattactgcatgcaggcactccgagcccctacttttggcggagggaccaaggttgagatcaaa

SEQ ID NO: 818 - protein, 111 aa, synthetic construct
  REGION 1..111: note = Synthetic: 47 - VL Protein
DIVMTQSPLSLPVTPGEPASISCRSSQSLLHSNGYNYLDWYLQKPGQSPQLLIYLGSHRASGVPDRFSGSGSGTDFTLKISRVEAEDVGVYYCMQALRAPTFGGGTKVEIK

SEQ ID NO: 819-900 - skipped (82 entries)

SEQ ID NO: 901 - protein, 9 aa, synthetic construct
  REGION 1..9: note = Synthetic: 46 - VH CDR1
FTFGDYAMS

SEQ ID NO: 902 - protein, 19 aa, synthetic construct
  REGION 1..19: note = Synthetic: 46 - VH CDR2
FIGSKAYGGTTEYTASVKG

SEQ ID NO: 903 - protein, 13 aa, synthetic construct
  REGION 1..13: note = Synthetic: 46 - VH CDR3
ARGPRRYTYGMDV

SEQ ID NO: 904 - protein, 11 aa, synthetic construct
  REGION 1..11: note = Synthetic: 46 - VL CDR1
RASQSISSYLN

SEQ ID NO: 905 - protein, 7 aa, synthetic construct
  REGION 1..7: note = Synthetic: 46 - VL CDR2
AASSLQS

SEQ ID NO: 906 - protein, 8 aa, synthetic construct
  REGION 1..8: note = Synthetic: 46 - VL CDR3
QQSSTPLT

SEQ ID NO: 907 - protein, 26 aa, synthetic construct
  REGION 1..26: note = Synthetic: 46 - VH FR1
EVQLVESGGGLVQPGRSLRLSCTASG

SEQ ID NO: 908 - protein, 14 aa, synthetic construct
  REGION 1..14: note = Synthetic: 46 - VH FR2
WFRQAPGKGLEWVG

SEQ ID NO: 909 - protein, 30 aa, synthetic construct; identical to SEQ ID NO: 309
  REGION 1..30: note = Synthetic: 46 - VH FR3

SEQ ID NO: 910 - protein, 11 aa, synthetic construct
  REGION 1..11: note = Synthetic: 46 - VH FR4
WGQGTTVTVSS

SEQ ID NO: 911 - DNA, 366 bp, synthetic construct
  misc_feature 1..366: note = Synthetic: 46 - VH DNA
  source 1..366: mol_type = other DNA
gaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattggaagcaaagcttatggtgggacaacagaatacaccgcgtctgtgaaaggcagattcaccatctcaagagatggttccaaaagcatcgcctatctgcaaatgaacagcctgaaaaccgaggacacggcggtgtactactgcgccagaggaccaagacgctacacatacggaatggacgtatggggccagggaacaactgtcaccgtctcctca

SEQ ID NO: 912 - protein, 122 aa, synthetic construct
  REGION 1..122: note = Synthetic: 46 - VH Protein
EVQLVESGGGLVQPGRSLRLSCTASGFTFGDYAMSWFRQAPGKGLEWVGFIGSKAYGGTTEYTASVKGRFTISRDGSKSIAYLQMNSLKTEDTAVYYCARGPRRYTYGMDVWGQGTTVTVSS

SEQ ID NO: 913 - protein, 23 aa, synthetic construct
  REGION 1..23: note = Synthetic: 46 - VL FR1
DIQMTQSPSSLSASVGDRVTITC

SEQ ID NO: 914 - protein, 15 aa, synthetic construct
  REGION 1..15: note = Synthetic: 46 - VL FR2
WYQQKPGKAPKLLIY

SEQ ID NO: 915 - protein, 32 aa, synthetic construct; identical to SEQ ID NO: 315
  REGION 1..32: note = Synthetic: 46 - VL FR3

SEQ ID NO: 916 - protein, 10 aa, synthetic construct
  REGION 1..10: note = Synthetic: 46 - VL FR4
FGGGTKVEIK

SEQ ID NO: 917 - DNA, 318 bp, synthetic construct; identical to SEQ ID NO: 317
  misc_feature 1..318: note = Synthetic: 46 - VL DNA
  source 1..318: mol_type = other DNA

SEQ ID NO: 918 - protein, 106 aa, synthetic construct; identical to SEQ ID NO: 318
  REGION 1..106: note = Synthetic: 46 - VL Protein

SEQ ID NO: 919-1000 - skipped (82 entries)

SEQ ID NO: 1001 - protein, 9 aa, synthetic construct
  REGION 1..9: note = Synthetic: 32 - VH CDR1
GTFSSYAIS

SEQ ID NO: 1002 - protein, 17 aa, synthetic construct
  REGION 1..17: note = Synthetic: 32 - VH CDR2
GIIPISGTANYAQKFQG

SEQ ID NO: 1003 - protein, 16 aa, synthetic construct
  REGION 1..16: note = Synthetic: 32 - VH CDR3
ARDTGRGYTRHFWFDP

SEQ ID NO: 1004 - protein, 11 aa, synthetic construct
  REGION 1..11: note = Synthetic: 32 - VL CDR1
RASQSISSYLN

SEQ ID NO: 1005 - protein, 7 aa, synthetic construct
  REGION 1..7: note = Synthetic: 32 - VL CDR2
AASSLQS

SEQ ID NO: 1006 - protein, 8 aa, synthetic construct
  REGION 1..8: note = Synthetic: 32 - VL CDR3
QQSDILYT

SEQ ID NO: 1007 - protein, 26 aa, synthetic construct
  REGION 1..26: note = Synthetic: 32 - VH FR1
QVQLVQSGAEVKKPGSSVKVSCKASG

SEQ ID NO: 1008 - protein, 14 aa, synthetic construct
  REGION 1..14: note = Synthetic: 32 - VH FR2
WVRQAPGQGLEWMG

SEQ ID NO: 1009 - protein, 30 aa, synthetic construct; identical to SEQ ID NO: 209
  REGION 1..30: note = Synthetic: 32 - VH FR3

SEQ ID NO: 1010 - protein, 11 aa, synthetic construct
  REGION 1..11: note = Synthetic: 32 - VH FR4
WGQGTLVTVSS

SEQ ID NO: 1011 - DNA, 369 bp, synthetic construct
  misc_feature 1..369: note = Synthetic: 32 - VH DNA
  source 1..369: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctctggtacagcaaactacgcacagaagttccagggcagagtcacgattaccgcggacgaatccacgagcacagcctacatggagctgagcagcctgagatctgaggacacggcggtgtactactgcgccagagacacgggacggggatacaccagacacttctggtttgacccctggggacagggtacattggtcaccgtctcctca

SEQ ID NO: 1012 - protein, 123 aa, synthetic construct
  REGION 1..123: note = Synthetic: 32 - VH Protein
QVQLVQSGAEVKKPGSSVKVSCKASGGTFSSYAISWVRQAPGQGLEWMGGIIPISGTANYAQKFQGRVTITADESTSTAYMELSSLRSEDTAVYYCARDTGRGYTRHFWFDPWGQGTLVTVSS

SEQ ID NO: 1013 - protein, 23 aa, synthetic construct
  REGION 1..23: note = Synthetic: 32 - VL FR1
DIQMTQSPSSLSASVGDRVTITC

SEQ ID NO: 1014 - protein, 15 aa, synthetic construct
  REGION 1..15: note = Synthetic: 32 - VL FR2
WYQQKPGKAPKLLIY

SEQ ID NO: 1015 - protein, 32 aa, synthetic construct; identical to SEQ ID NO: 315
  REGION 1..32: note = Synthetic: 32 - VL FR3

SEQ ID NO: 1016 - protein, 10 aa, synthetic construct
  REGION 1..10: note = Synthetic: 32 - VL FR4
FGGGTKVEIK

SEQ ID NO: 1017 - DNA, 318 bp, synthetic construct; identical to SEQ ID NO: 717
  misc_feature 1..318: note = Synthetic: 32 - VL DNA
  source 1..318: mol_type = other DNA

SEQ ID NO: 1018 - protein, 106 aa, synthetic construct; identical to SEQ ID NO: 718
  REGION 1..106: note = Synthetic: 32 - VL Protein

SEQ ID NO: 1019-2000 - skipped (982 entries)

SEQ ID NO: 2001 - protein, 9 aa, synthetic construct
  REGION 1..9: note = Synthetic: 33 - VH CDR1
GTFGNYAIS

SEQ ID NO: 2002 - protein, 17 aa, synthetic construct
  REGION 1..17: note = Synthetic: 33 - VH CDR2
GIIPIPGIANYAQKFQG

SEQ ID NO: 2003 - protein, 16 aa, synthetic construct
  REGION 1..16: note = Synthetic: 33 - VH CDR3
ARDTGRGYTRHFWFDP

SEQ ID NO: 2004 - protein, 11 aa, synthetic construct
  REGION 1..11: note = Synthetic: 33 - VL CDR1
RASQSISSYLN

SEQ ID NO: 2005 - protein, 7 aa, synthetic construct
  REGION 1..7: note = Synthetic: 33 - VL CDR2
AASSLQS

SEQ ID NO: 2006 - protein, 8 aa, synthetic construct
  REGION 1..8: note = Synthetic: 33 - VL CDR3
QQSDILYT

SEQ ID NO: 2007 - protein, 26 aa, synthetic construct
  REGION 1..26: note = Synthetic: 33 - VH FR1
QVQLVQSGAEVKKPGSSVKVSCKASG

SEQ ID NO: 2008 - protein, 14 aa, synthetic construct
  REGION 1..14: note = Synthetic: 33 - VH FR2
WVRQAPGQGLEWMG

SEQ ID NO: 2009 - protein, 30 aa, synthetic construct; identical to SEQ ID NO: 209
  REGION 1..30: note = Synthetic: 33 - VH FR3

SEQ ID NO: 2010 - protein, 11 aa, synthetic construct
  REGION 1..11: note = Synthetic: 33 - VH FR4
WGQGTLVTVSS

SEQ ID NO: 2011 - DNA, 369 bp, synthetic construct
  misc_feature 1..369: note = Synthetic: 33 - VH DNA
  source 1..369: mol_type = other DNA
caggtgcagctggtgcagtctggggctgaggtgaagaagcctgggtcctcggtgaaggtctcctgcaaggcttctggaggcaccttcggaaactatgctatcagctgggtgcgacaggcccctggacaagggcttgagtggatgggagggatcatccctatcccaggtatcgcaaactacgcacagaagttccagggcagagtcacgattaccgcggacgaatccacgagcacagcctacatggagctgagcagcctgagatctgaggacacggcggtgtactactgcgccagagacacgggacggggatacaccagacacttctggtttgacccctggggacagggtacattggtcaccgtctcctca

SEQ ID NO: 2012 - protein, 123 aa, synthetic construct
  REGION 1..123: note = Synthetic: 33 - VH Protein
QVQLVQSGAEVKKPGSSVKVSCKASGGTFGNYAISWVRQAPGQGLEWMGGIIPIPGIANYAQKFQGRVTITADESTSTAYMELSSLRSEDTAVYYCARDTGRGYTRHFWFDPWGQGTLVTVSS

SEQ ID NO: 2013 - protein, 23 aa, synthetic construct
  REGION 1..23: note = Synthetic: 33 - VL FR1
DIQMTQSPSSLSASVGDRVTITC

SEQ ID NO: 2014 - protein, 15 aa, synthetic construct
  REGION 1..15: note = Synthetic: 33 - VL FR2
WYQQKPGKAPKLLIY

SEQ ID NO: 2015 - protein, 32 aa, synthetic construct; identical to SEQ ID NO: 315
  REGION 1..32: note = Synthetic: 33 - VL FR3

SEQ ID NO: 2016 - protein, 10 aa, synthetic construct
  REGION 1..10: note = Synthetic: 33 - VL FR4
FGGGTKVEIK

SEQ ID NO: 2017 - DNA, 318 bp, synthetic construct; identical to SEQ ID NO: 717
  misc_feature 1..318: note = Synthetic: 33 - VL DNA
  source 1..318: mol_type = other DNA

SEQ ID NO: 2018 - protein, 106 aa, synthetic construct; identical to SEQ ID NO: 718
  REGION 1..106: note = Synthetic: 33 - VL Protein

SEQ ID NO: 2019-3000 - skipped (982 entries)

SEQ ID NO: 3001 - protein, 9 aa, synthetic construct
  REGION 1..9: note = Synthetic: 34 - VH CDR1
GTFSSAAIS

SEQ ID NO: 3002 - protein, 17 aa, synthetic construct
  REGION 1..17: note = Synthetic: 34 - VH CDR2
GIFPISGHANYAQKFQG

SEQ ID NO: 3003 - protein, 16 aa, synthetic construct
  REGION 1..16: note = Synthetic: 34 - VH CDR3
ARDTGRGYTRHFWFDP

SEQ ID NO: 3004 - protein, 11 aa, synthetic construct
  REGION 1..11: note = Synthetic: 34 - VL CDR1
RASQSISSYLN

SEQ ID NO: 3005 - protein, 7 aa, synthetic construct
  REGION 1..7: note = Synthetic: 34 - VL CDR2
AASSLQS

SEQ ID NO: 3006 - protein, 8 aa, synthetic construct
  REGION 1..8: note = Synthetic: 34 - VL CDR3
QQSDILYT

SEQ ID NO: 3007 - protein, 26 aa, synthetic construct
  REGION 1..26: note = Synthetic: 34 - VH FR1
QVQLVQSGAEVKKPGSSVKVSCKASG

SEQ ID NO: 3008 - protein, 14 aa, synthetic construct
  REGION 1..14: note = Synthetic: 34 - VH FR2
WVRQAPGQGLEWMG

SEQ ID NO: 3009 - protein, 30 aa, synthetic construct; identical to SEQ ID NO: 209
  REGION 1..30: note = Synthetic: 34 - VH FR3

SEQ ID NO: 3010 - protein, 11 aa, synthetic construct
  REGION 1..11: note = Synthetic: 34 - VH FR4
WGQGTLVTVSS

SEQ ID NO: 3011 - DNA, 369 bp, synthetic construct
  misc_feature 1..369: note = Synthetic: 34 - VH DNA
  source 1..369: mol_type = other DNA
caggtgcagctggtgcagtctggggctgaggtgaagaagcctgggtcctcggtgaaggtctcctgcaaggcttctggaggcaccttcagcagcgccgctatcagctgggtgcgacaggcccctggacaagggctcgagtggatgggagggatcttccctatctccggtcacgcaaactacgcacagaagttccagggcagagtcacgattaccgcggacgaatccacgagcacagcctacatggagctgagcagcctgagatctgaggacacggcggtgtactactgcgccagagacacgggacggggatacaccagacacttctggtttgacccctggggacagggtacattggtcaccgtctcctca

SEQ ID NO: 3012 - protein, 123 aa, synthetic construct
  REGION 1..123: note = Synthetic: 34 - VH Protein
QVQLVQSGAEVKKPGSSVKVSCKASGGTFSSAAISWVRQAPGQGLEWMGGIFPISGHANYAQKFQGRVTITADESTSTAYMELSSLRSEDTAVYYCARDTGRGYTRHFWFDPWGQGTLVTVSS

SEQ ID NO: 3013 - protein, 23 aa, synthetic construct
  REGION 1..23: note = Synthetic: 34 - VL FR1
DIQMTQSPSSLSASVGDRVTITC

SEQ ID NO: 3014 - protein, 15 aa, synthetic construct
  REGION 1..15: note = Synthetic: 34 - VL FR2
WYQQKPGKAPKLLIY

SEQ ID NO: 3015 - protein, 32 aa, synthetic construct; identical to SEQ ID NO: 315
  REGION 1..32: note = Synthetic: 34 - VL FR3

SEQ ID NO: 3016 - protein, 10 aa, synthetic construct
  REGION 1..10: note = Synthetic: 34 - VL FR4
FGGGTKVEIK

SEQ ID NO: 3017 - DNA, 318 bp, synthetic construct; identical to SEQ ID NO: 717
  misc_feature 1..318: note = Synthetic: 34 - VL DNA
  source 1..318: mol_type = other DNA

SEQ ID NO: 3018 - protein, 106 aa, synthetic construct; identical to SEQ ID NO: 718
  REGION 1..106: note = Synthetic: 34 - VL Protein

SEQ ID NO: 3019-4000 - skipped (982 entries)

SEQ ID NO: 4001 - protein, 9 aa, synthetic construct
  REGION 1..9: note = Synthetic: 36 - VH CDR1
GTFATYAIS

SEQ ID NO: 4002 - protein, 17 aa, synthetic construct
  REGION 1..17: note = Synthetic: 36 - VH CDR2
GIFPLSGTANYAQKFQG

SEQ ID NO: 4003 - protein, 16 aa, synthetic construct
  REGION 1..16: note = Synthetic: 36 - VH CDR3
ARDTGRGYTRHFWFDP

SEQ ID NO: 4004 - protein, 11 aa, synthetic construct
  REGION 1..11: note = Synthetic: 36 - VL CDR1
RASQSISSYLN

SEQ ID NO: 4005 - protein, 7 aa, synthetic construct
  REGION 1..7: note = Synthetic: 36 - VL CDR2
AASSLQS

SEQ ID NO: 4006 - protein, 8 aa, synthetic construct
  REGION 1..8: note = Synthetic: 36 - VL CDR3
QQSDILYT

SEQ ID NO: 4007 - protein, 26 aa, synthetic construct
  REGION 1..26: note = Synthetic: 36 - VH FR1
QVQLVQSGAEVKKPGSSVKVSCKASG

SEQ ID NO: 4008 - protein, 14 aa, synthetic construct
  REGION 1..14: note = Synthetic: 36 - VH FR2
WVRQAPGQGLEWMG

SEQ ID NO: 4009 - protein, 30 aa, synthetic construct; identical to SEQ ID NO: 209
  REGION 1..30: note = Synthetic: 36 - VH FR3

SEQ ID NO: 4010 - protein, 11 aa, synthetic construct
  REGION 1..11: note = Synthetic: 36 - VH FR4
WGQGTLVTVSS

SEQ ID NO: 4011 - DNA, 369 bp, synthetic construct
  misc_feature 1..369: note = Synthetic: 36 - VH DNA
  source 1..369: mol_type = other DNA
caggtgcagctggtgcagtctggggctgaggtgaagaagcctgggtcctcggtgaaggtctcctgcaaggcttctggaggcaccttcgcaacctatgctatcagctgggtgcgacaggcccctggacaagggcttgagtggatgggagggatcttccctctctccggtacagcaaactacgcacagaagttccagggcagagtcacgattaccgcggacgaatccacgagcacagcctacatggagctgagcagcctgagatctgaggacacggcggtgtactactgcgccagagacacgggacggggatacaccagacacttctggtttgacccctggggacagggtacattggtcaccgtctcctca

SEQ ID NO: 4012 - protein, 123 aa, synthetic construct
  REGION 1..123: note = Synthetic: 36 - VH Protein
QVQLVQSGAEVKKPGSSVKVSCKASGGTFATYAISWVRQAPGQGLEWMGGIFPLSGTANYAQKFQGRVTITADESTSTAYMELSSLRSEDTAVYYCARDTGRGYTRHFWFDPWGQGTLVTVSS

SEQ ID NO: 4013 - protein, 23 aa, synthetic construct
  REGION 1..23: note = Synthetic: 36 - VL FR1
DIQMTQSPSSLSASVGDRVTITC

SEQ ID NO: 4014 - protein, 15 aa, synthetic construct
  REGION 1..15: note = Synthetic: 36 - VL FR2
WYQQKPGKAPKLLIY

SEQ ID NO: 4015 - protein, 32 aa, synthetic construct; identical to SEQ ID NO: 315
  REGION 1..32: note = Synthetic: 36 - VL FR3

SEQ ID NO: 4016 - protein, 10 aa, synthetic construct
  REGION 1..10: note = Synthetic: 36 - VL FR4
FGGGTKVEIK

SEQ ID NO: 4017 - DNA, 318 bp, synthetic construct; identical to SEQ ID NO: 717
  misc_feature 1..318: note = Synthetic: 36 - VL DNA
  source 1..318: mol_type = other DNA

SEQ ID NO: 4018 - protein, 106 aa, synthetic construct; identical to SEQ ID NO: 718
  REGION 1..106: note = Synthetic: 36 - VL Protein

(19) United States
(12) Patent Application Publication
(10) Pub. No.: US 2025/0051449 A1
(43) Pub. Date: Feb. 13, 2025

(54) Anti-CD112R Compositions and Methods

(71) Applicant: Surface Oncology, LLC, Cambridge, MA (US)

(72) Inventors: Bianka PRINZ, Lebanon, NH (US); Nadthakarn BOLAND, Lebanon, NH (US); Kevin SCHUTZ, Lebanon, NH (US); John BUKOWSKI, Lebanon, NH (US); Jennifer SYMONDS, Lebanon, NH (US); James MOHAN, Cambridge, MA (US); Marisella Panduro SICHEVA, Cambridge, MA (US)

(73) Assignee: Surface Oncology, LLC, Cambridge, MA (US)

(21) Appl. No.: 18/930,273

(22) Filed: Oct. 29, 2024

Related U.S. Application Data
(63) Continuation of application No. 17/531,380, filed on Nov. 19, 2021, now Pat. No. 12,162,941.
Application No. 17/531,380 is a continuation of application No. 17/261,463, filed on Jan. 19, 2021, now Pat. No. 11,214,619.
(60) Provisional application No. 62/701,065, filed on Jul. 20, 2018.
(60) Provisional application No. 62/844,958, filed on May 8, 2019.

(51) Int. Cl. C07K 16/28; A61K 39/00; A61K 45/06; A61P 35/00; A61P 37/04

(52) U.S. Cl. CPC C07K 16/2818; A61P 35/00; A61P 37/04; C07K 16/2803; C07K 16/2827; A61K 2039/505; A61K 2039/507; A61K 45/06; C07K 2317/52; C07K 2317/73; C07K 2317/74; C07K 2317/76; C07K 2317/92

(57) ABSTRACT

The invention provides anti-CD112R antibody compositions and their use in treating cancer.

### CROSS-REFERENCE TO RELATED APPLICATIONS

This application claims benefit of U.S. Provisional Application No. 62/701,065, filed Jul. 20, 2018, and U.S. Provisional Application No. 62/844,958, filed May 8, 2019, the contents of which are incorporated herein by reference in their entirety.

### FIELD OF THE INVENTION

Anti-CD112R antibodies are provided, as well as their use in enhancing, increasing, and sustaining an anti-tumor immune response, treating cancer, and enhancing CD226 interactions with CD112.

### BACKGROUND

Both the innate and adaptive arms of the immune system utilize highly specialized immune cells to patrol the body, searching for signs of malignancy. Innate immunity provides the first line of defense and a rapid response using mechanisms such as barriers and destructive peptides that are non-specific and naturally present. Natural killer (NK) cells are a type of lymphocyte that is part of the innate immune system and can recognize and destroy virally infected and tumor cells using granzymes stored in their cytoplasm.

Adaptive immunity develops over time in response to antigen and provides lasting immunity. Cytotoxic lymphocytes (CTLs), also known as CD8⁺ T cells are part of the adaptive immune response as they recognize virus and tumor derived antigens presented by antigen presenting cells (APCs). CTLs are activated by interaction with an APC such as a dendritic cell or macrophage. The APC presents the tumor antigens in the context of MHC molecules to the T cell receptor (TCR) on the T cell surface. During this cognate interaction, the APC provides a costimulatory signal which leads to T cell activation, T cell proliferation, and reduction or elimination of cells expressing the antigen via cytotoxic mechanisms.

Administration of anti-CD112R immunotherapy provides an opportunity to increase, enhance and sustain immune responses. CD112R is an inhibitory receptor primarily expressed by T cells and NK cells and competes for CD112 binding with the activating receptor CD226. The interaction of CD112 with CD112R is of higher affinity than with CD226 and thereby effectively regulates CD226 mediated cell activation. Anti-CD112R antibodies that block the interaction with CD112 limit inhibitory signaling directly downstream of CD112R while simultaneously promoting greater immune cell activation by increasing CD226 interactions with CD112. In in vitro studies, anti-CD112R antibodies have been shown to increase the proliferation, activation and cytotoxicity of immune effector cells.

CD112R mRNA expression is detected in a number of cancer tissues and based on predictive analysis using TCGA (The Cancer Genome Atlas) dataset. Its expression is strongest in tumors that are enriched for T and NK cells. In addition to being expressed on myeloid cells, the expression of the CD112R ligand, CD112, is routinely elevated on tumor cells of different cellular origins. Given these circumstances, engagement of CD112R on tumor infiltrating immune cells has a strong potential to negatively regulate local immune responses within the tumor microenvironment.

Therapeutic treatment with anti-CD112R antibodies thereby provides an opportunity to down modulate the inhibitory signaling that occurs putatively when CD112R expressing immune cells engage CD112 on tumor cells and/or myeloid cells within the tumor microenvironment and has the potential to enhance, increase and sustain anti-tumor immune responses.

### SUMMARY

In some embodiments, an isolated anti-CD112R antibody is provided. Such isolated anti-CD112R antibody binds to human CD112R, wherein said antibody blocks the binding interaction between human CD112 and human CD112R and does not block the binding interaction between mouse CD112 and mouse CD112R, wherein the antibody is optionally fully human or humanized.

In some embodiments, the disclosure provides an isolated antibody comprising:
(a) HCDR1 comprising the amino acid sequence of SEQ ID NO: 1; (b) HCDR2 comprising the amino acid sequence of SEQ ID NO: 2; (c) HCDR3 comprising the amino acid sequence of SEQ ID NO: 3; (d) LCDR1 comprising the amino acid sequence of SEQ ID NO: 4; (e) LCDR2 comprising the amino acid sequence of SEQ ID NO: 5; and (f) LCDR3 comprising the amino acid sequence of SEQ ID NO: 6; or
(a) HCDR1 comprising the amino acid sequence of SEQ ID NO: 101; (b) HCDR2 comprising the amino acid sequence of SEQ ID NO: 102; (c) HCDR3 comprising the amino acid sequence of SEQ ID NO: 103; (d) LCDR1 comprising the amino acid sequence of SEQ ID NO: 104; (e) LCDR2 comprising the amino acid sequence of SEQ ID NO: 105; and (f) LCDR3 comprising the amino acid sequence of SEQ ID NO: 106; or
(a) HCDR1 comprising the amino acid sequence of SEQ ID NO: 201; (b) HCDR2 comprising the amino acid sequence of SEQ ID NO: 202; (c) HCDR3 comprising the amino acid sequence of SEQ ID NO: 203; (d) LCDR1 comprising the amino acid sequence of SEQ ID NO: 204; (e) LCDR2 comprising the amino acid sequence of SEQ ID NO: 205; and (f) LCDR3 comprising the amino acid sequence of SEQ ID NO: 206; or
(a) HCDR1 comprising the amino acid sequence of SEQ ID NO: 301; (b) HCDR2 comprising the amino acid sequence of SEQ ID NO: 302; (c) HCDR3 comprising the amino acid sequence of SEQ ID NO: 303; (d) LCDR1 comprising the amino acid sequence of SEQ ID NO: 304; (e) LCDR2 comprising the amino acid sequence of SEQ ID NO: 305; and (f) LCDR3 comprising the amino acid sequence of SEQ ID NO: 306; or
(a) HCDR1 comprising the amino acid sequence of SEQ ID NO: 401; (b) HCDR2 comprising the amino acid sequence of SEQ ID NO: 402; (c) HCDR3 comprising the amino acid sequence of SEQ ID NO: 403; (d) LCDR1 comprising the amino acid sequence of SEQ ID NO: 404; (e) LCDR2 comprising the amino acid sequence of SEQ ID NO: 405; and (f) LCDR3 comprising the amino acid sequence of SEQ ID NO: 406; or
(a) HCDR1 comprising the amino acid sequence of SEQ ID NO: 501; (b) HCDR2 comprising the amino acid sequence of SEQ ID NO: 502; (c) HCDR3 comprising the amino acid sequence of SEQ ID NO: 503; (d) LCDR1 comprising the amino acid sequence of SEQ ID NO: 504; (e) LCDR2 comprising the amino acid sequence of SEQ ID NO: 505; and (f) LCDR3 comprising the amino acid sequence of SEQ ID NO: 506; or
(a) HCDR1 comprising the amino acid sequence of SEQ ID NO: 601; (b) HCDR2 comprising the amino acid sequence of SEQ ID NO: 602; (c) HCDR3 comprising the amino acid sequence of SEQ ID NO: 603; (d) LCDR1 comprising the amino acid sequence of SEQ ID NO: 604; (e) LCDR2 comprising the amino acid sequence of SEQ ID NO: 605; and (f) LCDR3 comprising the amino acid sequence of SEQ ID NO: 606; or
(a) HCDR1 comprising the amino acid sequence of SEQ ID NO: 701; (b) HCDR2 comprising the amino acid sequence of SEQ ID NO: 702; (c) HCDR3 comprising the amino acid sequence of SEQ ID NO: 703; (d) LCDR1 comprising the amino acid sequence of SEQ ID NO: 704; (e) LCDR2 comprising the amino acid sequence of SEQ ID NO: 705; and (f) LCDR3 comprising the amino acid sequence of SEQ ID NO: 706; or
(a) HCDR1 comprising the amino acid sequence of SEQ ID NO: 801; (b) HCDR2 comprising the amino acid sequence of SEQ ID NO: 802; (c) HCDR3 comprising the amino acid sequence of SEQ ID NO: 803; (d) LCDR1 comprising the amino acid sequence of SEQ ID NO: 804; (e) LCDR2 comprising the amino acid sequence of SEQ ID NO: 805; and (f) LCDR3 comprising the amino acid sequence of SEQ ID NO: 806; or
(a) HCDR1 comprising the amino acid sequence of SEQ ID NO: 901; (b) HCDR2 comprising the amino acid sequence of SEQ ID NO: 902; (c) HCDR3 comprising the amino acid sequence of SEQ ID NO: 903; (d) LCDR1 comprising the amino acid sequence of SEQ ID NO: 904; (e) LCDR2 comprising the amino acid sequence of SEQ ID NO: 905; and (f) LCDR3 comprising the amino acid sequence of SEQ ID NO: 906; or
(a) HCDR1 comprising the amino acid sequence of SEQ ID NO: 1001; (b) HCDR2 comprising the amino acid sequence of SEQ ID NO: 1002; (c) HCDR3 comprising the amino acid sequence of SEQ ID NO: 1003; (d) LCDR1 comprising the amino acid sequence of SEQ ID NO: 1004; (e) LCDR2 comprising the amino acid sequence of SEQ ID NO: 1005; and (f) LCDR3 comprising the amino acid sequence of SEQ ID NO: 1006; or
(a) HCDR1 comprising the amino acid sequence of SEQ ID NO: 2001; (b) HCDR2 comprising the amino acid sequence of SEQ ID NO: 2002; (c) HCDR3 comprising the amino acid sequence of SEQ ID NO: 2003; (d) LCDR1 comprising the amino acid sequence of SEQ ID NO: 2004; (e) LCDR2 comprising the amino acid sequence of SEQ ID NO: 2005; and (f) LCDR3 comprising the amino acid sequence of SEQ ID NO: 2006; or
(a) HCDR1 comprising the amino acid sequence of SEQ ID NO: 3001; (b) HCDR2 comprising the amino acid sequence of SEQ ID NO: 3002; (c) HCDR3 comprising the amino acid sequence of SEQ ID NO: 3003; (d) LCDR1 comprising the amino acid sequence of SEQ ID NO: 3004; (e) LCDR2 comprising the amino acid sequence of SEQ ID NO: 3005; and (f) LCDR3 comprising the amino acid sequence of SEQ ID NO: 3006; or
(a) HCDR1 comprising the amino acid sequence of SEQ ID NO: 4001; (b) HCDR2 comprising the amino acid sequence of SEQ ID NO: 4002; (c) HCDR3 comprising the amino acid sequence of SEQ ID NO: 4003; (d) LCDR1 comprising the amino acid sequence of SEQ ID NO: 4004; (e) LCDR2 comprising the amino acid sequence of SEQ ID NO: 4005; and (f) LCDR3 comprising the amino acid sequence of SEQ ID NO: 4006.

In some embodiments, the antibody comprises a heavy chain variable region (VH) and a light chain variable region (VL), wherein:
the VH is at least 90%, 91%, 92%, 93%, 94%, 95%, 96%, 97%, 98%, 99%, or 100% identical to the amino acid sequence of SEQ ID NO: 12 and the VL is at least 90%, 91%, 92%, 93%, 94%, 95%, 96%, 97%, 98%, 99%, or 100% identical to the amino acid sequence of SEQ ID NO: 18; or
the VH is at least 90%, 91%, 92%, 93%, 94%, 95%, 96%, 97%, 98%, 99%, or 100% identical to the amino acid sequence of SEQ ID NO: 112 and the VL is at least 90%, 91%, 92%, 93%, 94%, 95%, 96%, 97%, 98%, 99%, or 100% identical to the amino acid sequence of SEQ ID NO: 118; or
the VH is at least 90%, 91%, 92%, 93%, 94%, 95%, 96%, 97%, 98%, 99%, or 100% identical to the amino acid sequence of SEQ ID NO: 212 and the VL is at least 90%, 91%, 92%, 93%, 94%, 95%, 96%, 97%, 98%, 99%, or 100% identical to the amino acid sequence of SEQ ID NO: 218; or
the VH is at least 90%, 91%, 92%, 93%, 94%, 95%, 96%, 97%, 98%, 99%, or 100% identical to the amino acid sequence of SEQ ID NO: 312 and the VL is at least 90%, 91%, 92%, 93%, 94%, 95%, 96%, 97%, 98%, 99%, or 100% identical to the amino acid sequence of SEQ ID NO: 318; or
the VH is at least 90%, 91%, 92%, 93%, 94%, 95%, 96%, 97%, 98%, 99%, or 100% identical to the amino acid sequence of SEQ ID NO: 412 and the VL is at least 90%, 91%, 92%, 93%, 94%, 95%, 96%, 97%, 98%, 99%, or 100% identical to the amino acid sequence of SEQ ID NO: 418; or
the VH is at least 90%, 91%, 92%, 93%, 94%, 95%, 96%, 97%, 98%, 99%, or 100% identical to the amino acid sequence of SEQ ID NO: 512 and the VL is at least 90%, 91%, 92%, 93%, 94%, 95%, 96%, 97%, 98%, 99%, or 100% identical to the amino acid sequence of SEQ ID NO: 518; or
the VH is at least 90%, 91%, 92%, 93%, 94%, 95%, 96%, 97%, 98%, 99%, or 100% identical to the amino acid sequence of SEQ ID NO: 612 and the VL is at least 90%, 91%, 92%, 93%, 94%, 95%, 96%, 97%, 98%, 99%, or 100% identical to the amino acid sequence of SEQ ID NO: 618; or
the VH is at least 90%, 91%, 92%, 93%, 94%, 95%, 96%, 97%, 98%, 99%, or 100% identical to the amino acid sequence of SEQ ID NO: 712 and the VL is at least 90%, 91%, 92%, 93%, 94%, 95%, 96%, 97%, 98%, 99%, or 100% identical to the amino acid sequence of SEQ ID NO: 718; or
the VH is at least 90%, 91%, 92%, 93%, 94%, 95%, 96%, 97%, 98%, 99%, or 100% identical to the amino acid sequence of SEQ ID NO: 812 and the VL is at least 90%, 91%, 92%, 93%, 94%, 95%, 96%, 97%, 98%, 99%, or 100% identical to the amino acid sequence of SEQ ID NO: 818; or
the VH is at least 90%, 91%, 92%, 93%, 94%, 95%, 96%, 97%, 98%, 99%, or 100% identical to the amino acid sequence of SEQ ID NO: 912 and the VL is at least 90%, 91%, 92%, 93%, 94%, 95%, 96%, 97%, 98%, 99%, or 100% identical to the amino acid sequence of SEQ ID NO: 918; or
the VH is at least 90%, 91%, 92%, 93%, 94%, 95%, 96%, 97%, 98%, 99%, or 100% identical to the amino acid sequence of SEQ ID NO: 1012 and the VL is at least 90%, 91%, 92%, 93%, 94%, 95%, 96%, 97%, 98%, 99%, or 100% identical to the amino acid sequence of SEQ ID NO: 1018; or
the VH is at least 90%, 91%, 92%, 93%, 94%, 95%, 96%, 97%, 98%, 99%, or 100% identical to the amino acid sequence of SEQ ID NO: 2012 and the VL is at least 90%, 91%, 92%, 93%, 94%, 95%, 96%, 97%, 98%, 99%, or 100% identical to the amino acid sequence of SEQ ID NO: 2018; or
the VH is at least 90%, 91%, 92%, 93%, 94%, 95%, 96%, 97%, 98%, 99%, or 100% identical to the amino acid sequence of SEQ ID NO: 3012 and the VL is at least 90%, 91%, 92%, 93%, 94%, 95%, 96%, 97%, 98%, 99%, or 100% identical to the amino acid sequence of SEQ ID NO: 3018; or
the VH is at least 90%, 91%, 92%, 93%, 94%, 95%, 96%, 97%, 98%, 99%, or 100% identical to the amino acid sequence of SEQ ID NO: 4012 and the VL is at least 90%, 91%, 92%, 93%, 94%, 95%, 96%, 97%, 98%, 99%, or 100% identical to the amino acid sequence of SEQ ID NO: 4018.

In some embodiments, the antibody comprises six CDRs (HCDR1, HCDR2, HCDR3, LCDR1, LCDR2, and LCDR3) as described herein, and VH and/or VL sequences that are at least 90%, 91%, 92%, 93%, 94%, 95%, 96%, 97%, 98%, 99%, or 100% identical to VH and/or VL amino acid sequence described herein. In some embodiments, the VH and/or VL sequences are not 100% identical to an amino acid sequence described herein. In some embodiments, the antibody comprises HCDR1, HCDR2, HCDR3, LCDR1, LCDR2, and LCDR3 amino acid sequences described herein, and sequence variation within the VH and/or VL sequences that is outside of the CDR sequences. In such embodiments, the sequence variation of the VH and/or VL sequences is within one or more framework regions of the VH and/or VL.

In some embodiments, the antibody comprises VH and/or VL sequences that are at least 90%, 91%, 92%, 93%, 94%, 95%, 96%, 97%, 98%, 99%, or 100% identical to an amino acid sequence described herein. In some embodiments, the VH and/or VL sequences are not 100% identical to an amino acid described herein. In such embodiments, the sequence variation of the VH and/or VL sequences is within and/or outside of the CDR sequences, unless otherwise specified.

In some embodiments, the antibody comprises a heavy chain variable region (VH) and a light chain variable region (VL), wherein:
the VH comprises the amino acid sequence of SEQ ID NO: 12 and the VL comprises the amino acid sequence of SEQ ID NO: 18; or
the VH comprises the amino acid sequence of SEQ ID NO: 112 and the VL comprises the amino acid sequence of SEQ ID NO: 118; or
the VH comprises the amino acid sequence of SEQ ID NO: 212 and the VL comprises the amino acid sequence of SEQ ID NO: 218; or
the VH comprises the amino acid sequence of SEQ ID NO: 312 and the VL comprises the amino acid sequence of SEQ ID NO: 318; or
the VH comprises the amino acid sequence of SEQ ID NO: 412 and the VL comprises the amino acid sequence of SEQ ID NO: 418; or
the VH comprises the amino acid sequence of SEQ ID NO: 512 and the VL comprises the amino acid sequence of SEQ ID NO: 518; or
the VH comprises the amino acid sequence of SEQ ID NO: 612 and the VL comprises the amino acid sequence of SEQ ID NO: 618; or
the VH comprises the amino acid sequence of SEQ ID NO: 712 and the VL comprises the amino acid sequence of SEQ ID NO: 718; or
the VH comprises the amino acid sequence of SEQ ID NO: 812 and the VL comprises the amino acid sequence of SEQ ID NO: 818; or
the VH comprises the amino acid sequence of SEQ ID NO: 912 and the VL comprises the amino acid sequence of SEQ ID NO: 918; or
the VH comprises the amino acid sequence of SEQ ID NO: 1012 and the VL comprises the amino acid sequence of SEQ ID NO: 1018; or
the VH comprises the amino acid sequence of SEQ ID NO: 2012 and the VL comprises the amino acid sequence of SEQ ID NO: 2018; or
the VH comprises the amino acid sequence of SEQ ID NO: 3012 and the VL comprises the amino acid sequence of SEQ ID NO: 3018; or
the VH comprises the amino acid sequence of SEQ ID NO: 4012 and the VL comprises the amino acid sequence of SEQ ID NO: 4018.

In some embodiments, anti-CD112R antibodies are provided that activate NK cells. In some embodiments, anti-CD112R antibodies are provided that upregulate CD137 on NK cells. In some embodiments, the anti-CD112R antibodies that activate NK cells and/or upregulate CD137 on NK cells are antibodies any of the antibodies described in the Table of Sequences, such as, for example, antibodies 32, 33, 34, 35, and 36. In some embodiments, the anti-CD112R antibodies that activate NK cells and/or upregulate CD137 on NK cells comprise the six CDRs of antibodies 32, 33, 34, 35, and 36 respectively (see Table of Sequences).

In some embodiments, the antibody is a monoclonal antibody. In some embodiments, the antibody is a humanized antibody. In some embodiments, the antibody is a fully human antibody. In some embodiments, the antibody is an antibody fragment selected from a Fab, Fab′, Fv, scFv or (Fab′)₂. In some embodiments, the antibody is a full-length antibody. In some embodiments, the isolated antibody comprises an IgG1, IgG2, IgG3, or IgG4 Fc region. In some embodiments, the disclosure provides a composition comprising an antibody disclosed herein and a pharmaceutically acceptable carrier.

In some embodiments, the antibody increases NK cell degranulation, increases activation of NK cells, increases activation of intra-tumoral NK cells when presented in combination with an anti-TIGIT antibody, inhibits tumor growth in vivo, and/or prevents tumor engraftment upon re-challenge with tumor. In some such embodiments, the antibody comprises a human IgG1 heavy chain constant region, and the antibody increases NK cell degranulation, increases activation of NK cells, increases activation of intra-tumoral NK cells when presented in combination with an anti-TIGIT antibody, inhibits tumor growth in vivo, and/or prevents tumor engraftment upon re-challenge with tumor relative to an otherwise identical antibody comprising a human IgG heavy chain constant region of a different isotype.

In some embodiments, the disclosure provides a method of enhancing, increasing and/or sustaining an anti-tumor immune response in a subject comprising administering the antibody or composition described herein to a subject having a tumor.

In some embodiments, the disclosure provides a method of treating cancer in a subject comprising administering the antibody or composition described herein to a subject having cancer. In some embodiments, the cancer is carcinoma, lymphoma, blastoma, sarcoma, or leukemia. In some embodiments, the cancer is squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck).

In some embodiments, the disclosure provides a method of enhancing CD226 interactions with CD112 in a subject comprising administering the antibody or the composition described herein to a subject.

In some embodiments, the disclosure provides a method of enhancing CD8 T cell activation in a subject comprising administering the antibody or the composition described herein to a subject in need of CD8 T cell activation.

In some embodiments, the disclosure provides a method of enhancing CD8 T cell interferon gamma production in a subject comprising administering the antibody or the composition described herein to a subject in need of CD8 T cell interferon gamma production.

In some embodiments, the disclosure provides a method of enhancing NK cell activation in a subject comprising administering the antibody or the composition described herein to a subject in need of NK cell activation.

In some embodiments, the disclosure provides a method of enhancing NK cell mediated cytotoxicity in a subject comprising administering the antibody or the composition described herein to a subject in need of increasing NK cell mediated cytotoxicity.

In some embodiments, the methods described herein further comprise administering a second therapy. In some embodiments, the second therapy is radiotherapy, surgery or administration of a second agent. In some embodiments, the second therapy is a second agent. In some such embodiments, the second agent is an antagonist of PD-1, PD-L1, CTLA-4, Lag-3 or TIM-3. In some embodiments, the second agent is an antagonist of TIGIT or CD96. In some embodiments, the second agent is an antagonist of PVRL1, PVRL2, PVRL3, PVRL4, or CD155. In some embodiments, the second agent an antagonist of CD47, CD39, or IL-27. In some embodiments, the second agent is a STING agonist.

In some embodiments, the disclosure provides use of the antibody or the composition described herein for enhancing, and/or increasing and/or sustaining an anti-tumor immune, and/or treating cancer, and/or enhancing CD226 interactions with CD112.

In some embodiments, the disclosure provides use of the antibody or the composition described herein in the preparation of a medicament for enhancing, and/or increasing and/or sustaining an anti-tumor immune response, and/or treating cancer, and/or enhancing CD226 interactions with CD112.

In some embodiments, the disclosure provides a nucleic acid encoding an antibody disclosed herein.

In some embodiments, the disclosure provides a host cell comprising a nucleic acid encoding an antibody disclosed herein.

In some embodiments, the disclosure provides a method of producing an antibody disclosed herein comprising culturing host cell comprising a nucleic acid encoding an antibody disclosed herein, the host cell being cultured under conditions wherein the antibody is expressed. In some embodiments, the method further comprises purifying the antibody.

Exemplary embodiments of the disclosure include the following:

Embodiment 1. An isolated anti-CD112R antibody which binds to human CD112R, wherein said antibody blocks the binding interaction between human CD112 and human CD112R and does not block the binding interaction between mouse CD112 and mouse CD112R, wherein the antibody is optionally fully human or humanized.

Embodiment 2. The isolated antibody of embodiment 1, wherein the isolated antibody comprises:
i) (a) HCDR1 comprising the amino acid sequence of SEQ ID NO: 701; (b) HCDR2 comprising the amino acid sequence of SEQ ID NO: 702; (c) HCDR3 comprising the amino acid sequence of SEQ ID NO: 703; (d) LCDR1 comprising the amino acid sequence of SEQ ID NO: 704; (e) LCDR2 comprising the amino acid sequence of SEQ ID NO: 705; and (f) LCDR3 comprising the amino acid sequence of SEQ ID NO: 706; or
ii) (a) HCDR1 comprising the amino acid sequence of SEQ ID NO: 1001; (b) HCDR2 comprising the amino acid sequence of SEQ ID NO: 1002; (c) HCDR3 comprising the amino acid sequence of SEQ ID NO: 1003; (d) LCDR1 comprising the amino acid sequence of SEQ ID NO: 1004; (e) LCDR2 comprising the amino acid sequence of SEQ ID NO: 1005; and (f) LCDR3 comprising the amino acid sequence of SEQ ID NO: 1006; or
iii) (a) HCDR1 comprising the amino acid sequence of SEQ ID NO: 2001; (b) HCDR2 comprising the amino acid sequence of SEQ ID NO: 2002; (c) HCDR3 comprising the amino acid sequence of SEQ ID NO: 2003; (d) LCDR1 comprising the amino acid sequence of SEQ ID NO: 2004; (e) LCDR2 comprising the amino acid sequence of SEQ ID NO: 2005; and (f) LCDR3 comprising the amino acid sequence of SEQ ID NO: 2006; or
iv) (a) HCDR1 comprising the amino acid sequence of SEQ ID NO: 3001; (b) HCDR2 comprising the amino acid sequence of SEQ ID NO: 3002; (c) HCDR3 comprising the amino acid sequence of SEQ ID NO: 3003; (d) LCDR1 comprising the amino acid sequence of SEQ ID NO: 3004; (e) LCDR2 comprising the amino acid sequence of SEQ ID NO: 3005; and (f) LCDR3 comprising the amino acid sequence of SEQ ID NO: 3006; or
v) (a) HCDR1 comprising the amino acid sequence of SEQ ID NO: 4001; (b) HCDR2 comprising the amino acid sequence of SEQ ID NO: 4002; (c) HCDR3 comprising the amino acid sequence of SEQ ID NO: 4003; (d) LCDR1 comprising the amino acid sequence of SEQ ID NO: 4004; (e) LCDR2 comprising the amino acid sequence of SEQ ID NO: 4005; and (f) LCDR3 comprising the amino acid sequence of SEQ ID NO: 4006; or
vi) (a) HCDR1 comprising the amino acid sequence of SEQ ID NO: 1001 with 1, 2, or 3 amino acid changes to positions 4, 5, and/or 6 of SEQ ID NO: 1001; (b) HCDR2 comprising the amino acid sequence of SEQ ID NO: 1002 with 1, 2, 3, 4, or 5 amino acid changes to positions 1, 3, 5, 6, and/or 8 of SEQ ID NO: 1002; (c) HCDR3 comprising the amino acid sequence of SEQ ID NO: 1003; (d) LCDR1 comprising the amino acid sequence of SEQ ID NO: 1004; (e) LCDR2 comprising the amino acid sequence of SEQ ID NO: 1005; and (f) LCDR3 comprising the amino acid sequence of SEQ ID NO: 1006.

Embodiment 3. The isolated antibody of embodiment 1 or 2, wherein the antibody comprises a heavy chain variable region (VH) and a light chain variable region (VL), wherein:
i) the VH is at least 90%, 91%, 92%, 93%, 94%, 95%, 96%, 97%, 98%, 99%, or 100% identical to the amino acid sequence of SEQ ID NO: 712 and the VL is at least 90%, 91%, 92%, 93%, 94%, 95%, 96%, 97%, 98%, 99%, or 100% identical to the amino acid sequence of SEQ ID NO: 718; or
ii) the VH is at least 90%, 91%, 92%, 93%, 94%, 95%, 96%, 97%, 98%, 99%, or 100% identical to the amino acid sequence of SEQ ID NO: 1012 and the VL is at least 90%, 91%, 92%, 93%, 94%, 95%, 96%, 97%, 98%, 99%, or 100% identical to the amino acid sequence of SEQ ID NO: 1018; or
iii) the VH is at least 90%, 91%, 92%, 93%, 94%, 95%, 96%, 97%, 98%, 99%, or 100% identical to the amino acid sequence of SEQ ID NO: 2012 and the VL is at least 90%, 91%, 92%, 93%, 94%, 95%, 96%, 97%, 98%, 99%, or 100% identical to the amino acid sequence of SEQ ID NO: 2018; or
iv) the VH is at least 90%, 91%, 92%, 93%, 94%, 95%, 96%, 97%, 98%, 99%, or 100% identical to the amino acid sequence of SEQ ID NO: 3012 and the VL is at least 90%, 91%, 92%, 93%, 94%, 95%, 96%, 97%, 98%, 99%, or 100% identical to the amino acid sequence of SEQ ID NO: 3018; or
v) the VH is at least 90%, 91%, 92%, 93%, 94%, 95%, 96%, 97%, 98%, 99%, or 100% identical to the amino acid sequence of SEQ ID NO: 4012 and the VL is at least 90%, 91%, 92%, 93%, 94%, 95%, 96%, 97%, 98%, 99%, or 100% identical to the amino acid sequence of SEQ ID NO: 4018, optionally with the proviso that if any sequence variation is present in the CDRs, such sequence variation is within HCDR1 or HCDR2 with no more than 3 amino acid changes, such as no more than 2 amino acid changes to positions 4, 5, and/or 6 of HCDR1 and no more than 5 amino acids changes, such as no more than 2 amino acid changes to positions 1, 3, 5, 6, and/or 8 of HCDR2, optionally wherein the variation is not within HCDR3, LCDR1, LCDR2 and LCDR3.

Embodiment 4. The isolated antibody of any one of embodiments 1-3, wherein the antibody comprises a heavy chain variable region (VH) and a light chain variable region (VL), wherein:
i) the VH comprises the amino acid sequence of SEQ ID NO: 712 and the VL comprises the amino acid sequence of SEQ ID NO: 718; or
ii) the VH comprises the amino acid sequence of SEQ ID NO: 1012 and the VL comprises the amino acid sequence of SEQ ID NO: 1018; or
iii) the VH comprises the amino acid sequence of SEQ ID NO: 2012 and the VL comprises the amino acid sequence of SEQ ID NO: 2018; or
iv) the VH comprises the amino acid sequence of SEQ ID NO: 3012 and the VL comprises the amino acid sequence of SEQ ID NO: 3018; or
v) the VH comprises the amino acid sequence of SEQ ID NO: 4012 and the VL comprises the amino acid sequence of SEQ ID NO: 4018.

Embodiment 5. The isolated antibody of any one of the preceding embodiments, wherein the antibody is a monoclonal antibody.

Embodiment 6. The isolated antibody of any one of the preceding embodiments, wherein the antibody is a full-length antibody or is an antibody fragment, optionally a Fab, Fab′, Fv, scFv or (Fab′)₂.

Embodiment 7. The isolated antibody of any one of embodiments 1-5, wherein the antibody comprises an IgG1, IgG2, IgG3, or IgG4 Fc region, wherein the antibody optionally comprises a human IgG1 heavy chain constant region, a human IgG4 heavy chain constant region, or a mutant human IgG4 heavy chain constant region, wherein the mutant human IgG4 heavy chain constant region optionally comprises a mutation selected from a substitution at Ser228, a substitution at Leu235, a substitution at Asn297, or a combination thereof, numbering according to EU numbering or an S228P substitution and an L235E substitution, numbering according to EU numbering.

Embodiment 8. The isolated antibody of any one of embodiments 1-7, wherein the antibody
i) increases NK cell degranulation; and/or
ii) increases activation of NK cells; and/or
iii) increases activation of intra-tumoral NK cells when presented in combination with an anti-TIGIT antibody; and/or
iv) inhibits tumor growth in vivo; and/or
v) prevents tumor engraftment upon re-challenge with tumor, optionally wherein the antibody is IgG1 or IgG4.

Embodiment 9. A pharmaceutical composition comprising the antibody of any one of embodiments 1-8 and a pharmaceutically acceptable carrier, wherein the composition optionally comprises an opsonizing agent, a regulatory T cell depleting agent, chemotherapy, and/or an antagonist of PD-1, PD-L1, CTLA-4, Lag-3 or TIM-3.

Embodiment 10. The isolated antibody of any one of embodiments 1-8 or pharmaceutical composition of embodiment 9 for use in enhancing, increasing and/or sustaining an anti-tumor immune response in a subject, optionally wherein CD8 T cell activation is enhanced or CD8 T cell interferon gamma production is enhanced in the subject, or optionally wherein NK cell activation is enhanced in the subject or NK cell mediated cytotoxicity is enhanced in the subject, or optionally wherein CD226 interactions with CD112 are enhanced in a subject.

Embodiment 11. The isolated antibody of any one of embodiments 1-8 or pharmaceutical composition of embodiment 9 for use in treating cancer in a subject, wherein the cancer is optionally carcinoma, lymphoma, blastoma, sarcoma, or leukemia, or wherein the cancer is optionally squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck).

Embodiment 12. The isolated antibody or pharmaceutical composition for use of embodiment 10 or 11, wherein the use further comprises administering a second therapy, wherein the second therapy is optionally radiotherapy, surgery or administration of a second agent, wherein the second agent is optionally an antagonist of PD-1, PD-L1, CTLA-4, Lag-3 or TIM-3, or an antagonist of TIGIT or CD96, or an antagonist of PVRL1, PVRL2, PVRL3, PVRL4, and CD155, or is an antagonist of CD47, or is an antagonist of CD39, or is an antagonist of IL-27, or is a STING agonist, wherein the second agent is optionally an antagonist antibody.

Embodiment 13. A nucleic acid encoding the antibody of any one of embodiments 1-8.

Embodiment 14. A host cell comprising the nucleic acid of embodiment 13.

Embodiment 15. A method of producing the antibody of any one of embodiment 1-8 comprising culturing the host cell of embodiment 14 under conditions wherein the antibody is expressed, optionally further comprising purifying the antibody.

### BRIEF DESCRIPTION OF THE FIGURES

FIG. 1 depicts the ability of anti-CD112R antibodies, as compared to an IgG1 isotype control antibody, to bind to Jurkat cells engineered to overexpress human CD112R. Binding intensity was assessed by the geometric mean fluorescent intensity (gMFI) of the Alexa Fluor® 647 antibody label.

FIGS. 2A-2B depict the ability of anti-CD112R antibodies, as compared to an IgG1 isotype control antibody, to block the interaction of CD112R with CD112 on Jurkat cells engineered to overexpress human CD112R. In FIG. 2A, cells were pre-incubated with either IgG1 isotype control or anti-CD112R antibody. After washing, cells were stained with biotinylated his-labeled human CD112 and Streptavidin-PE simultaneously. The ability of anti-CD112R antibodies to block the binding of CD112 to CD112R was assessed by the geometric mean fluorescent intensity (gMFI) of the PE labeling and displayed as percent inhibition. Percent inhibition was calculated as [100−((test sample MFI/Max MFI)*100%)].

FIG. 2B shows percent inhibition as measured by ELISA of the interaction between human CD112R and CD112 by anti-CD112R antibodies described herein.

FIG. 3 shows enhanced human NK cell mediated cytotoxicity against REH cells (human leukemia cell line) in the presence of anti-CD112R antibodies as compared to an IgG1 isotype antibody. Activated NK cells and cellTrace violet labeled REH cells were co-cultured for four hours. After co-culture, the viability of REH cells was assessed by staining with 7-AAD. Cytotoxicity (percent over isotype) was calculated as ((test percent dead minus isotype percent dead) divided by isotype percent dead)×100.

FIG. 4 shows enhanced antigen driven activation of CD8+ T cells, as measured by IFNγ secretion, in the presence of anti-CD112R antibodies, as compared to an IgG1 isotype control antibody. Colo205 cells were pulsed with pp65 peptide and co-cultured with human CMV specific T cells in the presence of anti-CD112R antibody or an IgG1 isotype control. IFNγ levels in the supernatants of cultured cells was measured by Luminex. CD8+ T cells treated with anti-CD112R antibodies 2 and 5 resulted in greater IFNγ secretion than observed with isotype control.

FIG. 5 is a graph depicting that a combination of murine anti-CD112R and murine anti-TIGIT antibodies has therapeutic effect in the mouse syngeneic CT-26 tumor model. Tumor-bearing mice were randomized into four groups and treated twice weekly for two weeks, by IP injection, with 1) isotype control antibody; 2) anti-TIGIT antibody; 3) anti-CD112R antibody; or 4) anti-TIGIT antibody combined with anti-CD112R antibody. Mean tumor volumes for each treatment group are depicted as a function of time. The results demonstrate that the combination of anti-CD112R with anti-TIGIT was effective at reducing tumor growth compared to isotype treated animals while anti-CD112R or anti-TIGIT monotherapies showed either no activity or only a modest effect on reducing tumor growth.

FIG. 6 is a graph depicting increased expression of CD112R in PBMCs following anti-CD3 activation. Human PBMCs were stimulated in vitro with anti-CD3 antibody and CD112R expression was assessed by flow cytometry. Quantitation of CD112R antibody binding was assessed by the geometric mean fluorescent intensity (gMFI) of the Alexa Fluor® 647 antibody label for the indicated cell type. CD112R is depicted as fold change over the negative control (FON, (CD112R gMFI divided by isotype gMFI)).

FIG. 7 shows enhanced NK cell mediated degranulation in response to tumor cells in the presence of CD112R antibodies with an IgG1 isotype. Human NK cells and Raji.CD112 cells were co-cultured for four hours with CD107a PE antibody in the presence of CD112R antibodies. After co-culture, NK cell degranulation was determined by frequency of NK cells that were CD107a positive.

FIG. 8A-8D show enhanced NK cell activation in the presence of a CD112R antibody with an IgG1 isotype. Human PBMCs from 2 different donors and K562 cells were co-cultured for 16 hours in the presence of a CD112R antibody. After co-culture, NK cell activation was determined by the frequency of NK cells that were CD137 positive. The results for donor 1 and donor 2 in two independent assays are shown in FIGS. 8A-8D, respectively.

FIG. 9 shows tumor growth inhibition in mice treated with an anti-CD112R antibody. The figure shows a summary of 3 experiments, N=44-45 per group. Statistical analysis was performed by Mann-Whitney test on day 24 post implant.

FIG. 10A-10B show that treatment with anti-CD112R antibody increases the overall survival of mice inoculated with CT-26 tumors and protects mice from tumor rechallenge. FIG. 10A shows the survival frequency of mice following primary tumor challenge with anti-CD112R treatment. Survivor mice exhibited no palpable tumors beyond day 50 of inoculation and were deemed to be complete responders. FIG. 10B shows tumor growth inhibition in survival mice upon tumor rechallenge compared to naïve control mice. Statistical analysis was performed by Mantel-Cox test on day 50 post implant (FIG. 10A) and by Mann-Whitney test on day 15 post implant (FIG. 10B).

FIG. 11 shows the in vivo efficacy of CD112R blockade in a CT26 mouse tumor model is dependent on NK cells and CD8 T cells. The figure shows tumor growth inhibition of anti-CD112R treated mice simultaneously depleted of either NK cells or CD8 T cells.

FIG. 12A-12B show expression of CD69 (FIG. 12A) and Granzyme B (FIG. 12B) on intratumoral NK cells in CT-26 tumor model after treatment with an anti-CD112R antibody.

FIG. 13A-13F show mean (FIG. 13A) and individual (FIGS. 13B-E) tumor volume measurements as a function of time in a CT-26 tumor model after administration of anti-CD112R antibodies alone and in combination with anti-PD1 antibodies. FIG. 13F shows overall tumor-free survival on day 50 post-implantation as a fraction of tumor-free survivors per group.

FIG. 14A-14F show alignments of the CDR sequences of antibodies described herein. FIGS. 14A-14C show the CDR sequences of the heavy chain variable region, and FIGS. 14D-14F show the CDR sequences of the light chain variable region. In each of FIGS. 14A-14F, the first column shows HCDR1 (FIG. 14A), HCDR2 (FIG. 14B), and HCDR3 (FIG. 14C), LCDR1 (FIG. 14D), LCDR2 (FIG. 14E), and LCDR3 (FIG. 14F), where the antibody clone number is provided preceding H1 (for HCDR1), H2 (for HCDR2), H3 (for HCDR3). L1 (for LCDR1). L2 (for LCDR2), and L3 (for LCDR3). The second column shows the sequence, the third column shows the number of amino acids in the sequence, and the last column shows the percent identity of each sequence relative to the sequence from parent antibody clone 32. Family member clones 32, 33, 34, 35, and 36 are bolded.

FIG. 15A-15H show alignments of the framework region sequences of antibodies described herein. FIGS. 15A-15D show the framework region sequences of the heavy chain variable region, and FIGS. 15E-15H show the framework region sequences of the light chain variable region. In each of FIGS. 15A-15F, the first column shows heavy chain FR1 (FIG. 15A). FR2 (FIG. 15B). FR3 (FIG. 15C). FR4 (FIG. 15D) and light chain FR1 (FIG. 15E). FR2 (FIG. 15F), and FR3 (FIG. 15G), and FR4 (FIG. 15H), where the antibody clone number is provided preceding VH (for HFR1. HFR3. HFR3, and HFR4), and VL (for LFR1. LFR2. LFR3, and LFR4). The second column shows the sequence, and the last column shows the percent identity of each sequence relative to the sequence from parent antibody clone 32. Family member clones 32, 33, 34, 35, and 36 are bolded.

FIG. 16A-16B show alignments of the variable region sequences of antibodies described herein. FIG. 16A shows the heavy chain variable region sequences, and FIG. 16B shows the light chain variable region sequences. Each sequence is labeled with its corresponding clone number. The percent identity of each sequence relative to the sequence from antibody clone 32 is shown. Family member clones 32, 33, 34, 35, and 36 are bolded.

FIG. 17 shows extent of binding of anti-CD112R antibodies described herein and additional anti-CD112R antibodies (antibodies A. B, and C that bind human CD112R) to cells expressing mouse CD112R.

FIG. 18 shows extent of binding of anti-CD112R antibodies described herein and additional anti-CD112R antibodies (antibodies A, B, and C that bind human CD112R) to soluble mouse CD112R.

FIG. 19 shows percent inhibition of the interaction between mouse CD11R and mouse CD112 by anti-CD112R antibodies described herein and additional anti-CD112R antibodies (antibodies A, B, and C that bind human CD112R).

### DETAILED DESCRIPTION OF EMBODIMENTS OF THE INVENTION

### I. Definitions

In this application, the use of “or” means “and/or” unless stated otherwise. In the context of a multiple dependent claim, the use of “or” refers back to more than one preceding independent or dependent claim in the alternative only. The terms “comprising.” “including.” and “having” can be used interchangeably herein.

The terms “CD112R,” “PVR Related Immunoglobulin Domain Containing,” “CD112 Receptor,” “Poliovirus Receptor-Related Immunoglobulin Domain-Containing Protein” “Poliovirus Receptor Related Immunoglobulin Domain Containing,” “Nectin-2 Receptor,” “C7orf15,” and “Transmembrane Protein PVRIG” are all used interchangeably and refer to a native, human CD112R, unless otherwise specifically indicated (e.g. mouse CD112R, cynomolgus CD112R, etc.). The term includes full-length, unprocessed CD112R as well as any form of CD112R that results from processing in the cell. The term encompasses naturally occurring variants of human CD112R, e.g., splice variants or allelic variants. External ID's for CD112R gene include Entrez Gene: 79037, Ensembl: ENSG00000213413, OMIM: 617012, and UniProtKB: Q6DKI7.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations optionally resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The term “block,” in the context of an interaction between two or more molecules, is used herein to refer to inhibition or prevention of said interaction between the two or more molecules, wherein the inhibition or prevention of said interaction between the two or more molecules is complete or nearly complete under at least one condition. A “nearly complete” inhibition is a percent inhibition of about 70-99.9%, and a “complete” inhibition is 100%. For example, a molecule is said to “block” an interaction between two or more other molecules if it completely or nearly completely inhibits such interaction at certain concentrations in a dose dependent manner.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “tumor” is used herein to refer to a cell or cells that comprise a cancer. The term “tumor growth” is used herein to refer to proliferation or growth by a cell or cells that comprise a cancer that leads to a corresponding increase in the size or extent of the cancer.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

Administration “in combination with” one or more further therapeutic agents includes simultaneous (concurrent) and consecutive (sequential) administration in any order.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present (numbering in this paragraph is according to the EU numbering system, also called the EU index, as described in Kabat et al., Sequences of Proteins of Immunological Interest, 5th Ed. Public Health Service, National Institutes of Health, Bethesda, MD, 1991).

“Framework,” “framework region,” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See. e.g., Portolano et al., J. Immunol. 150:880-887 (1993); Clarkson et al., Nature 352:624-628 (1991).

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al . . . supra. In some embodiments, for the VH, the subgroup is subgroup III as in Kabat et al., supra.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3).

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

Anti-CD112R antibodies, compositions comprising the described antibodies and methods of their use are provided.

### A. Exemplary Anti-CD112R Antibodies

The Sequence Table below provides the sequences of certain embodiments of the antibodies disclosed and claimed herein.

In certain embodiments, antibodies are provided that bind to CD112R, and/or block binding of CD112R to CD112, and/or enhance activation of T cells and NK cells. In some embodiments, antibodies are provided that bind to CD112R. In some embodiments, antibodies are provided that block CD112R binding to CD112. In some embodiments, antibodies are provided that enhance activation of CD226, T cells, and/or NK cells.

Inhibition of binding between CD112R and CD112 such as on T and NK cells can be determined by measuring the inhibition of binding of cells to which CD112R binds in the presence and absence of the antibody.

Provided herein are antibodies that bind specifically to CD112R.

In some embodiments, the antibodies bind to human CD112R.

In some embodiments, the antibodies bind to human CD112R, and block the interaction of human CD112R to human CD112. In some embodiments, the antibodies bind to human CD112R, block the interaction of human CD112R to human CD112, but do not block the interaction of mouse CD112R to mouse CD112. In some embodiments, the antibodies that bind to human CD112R, block the interaction of human CD112R to human CD112, but do not block the interaction of mouse CD112R to mouse CD112 comprise antibodies 32, 33, 34, 35, and 36.

In certain embodiments, a CD112R antibody comprises a heavy chain variable region (“VH”) comprising VH CDR1, CDR2 and/or CDR3 of any of the CD112R antibodies provided herein (i.e., antibody clone numbers 2, 5, 44, 58, 10, 38, 15, 35, 46, 47, 32, 33, 34, or 36).

In certain embodiments, a CD112R antibody comprises a VH comprising VH CDR1, CDR2 and/or CDR3 of any of the CD112R antibodies provided herein and a VL comprising CDR1, CDR2 and/or CDR3 of any of the CD112R antibodies provided herein. In certain embodiments, a CD112R antibody comprises a VH comprising VH CDR1, CDR2 and/or CDR3 of any one of antibody clone numbers 2, 5, 44, 58, 10, 38, 15, 35, 46, 47, 32, 33, 34, or 36, and a VL comprising VL CDR1, CDR2, and/or CDR3 of any one of antibody clone numbers 2, 5, 44, 58, 10, 38, 15, 35, 46, 47, 32, 33, 34, or 36, optionally wherein the VH and VL CDRs are from the same antibody clone.

In some embodiments, antibodies comprising the following are provided:
(a) HCDR1 comprising the amino acid sequence of SEQ ID NO: 1; (b) HCDR2 comprising the amino acid sequence of SEQ ID NO: 2; (c) HCDR3 comprising the amino acid sequence of SEQ ID NO: 3; with or without (d) LCDR1 comprising the amino acid sequence of SEQ ID NO: 4; (e) LCDR2 comprising the amino acid sequence of SEQ ID NO: 5; and (f) LCDR3 comprising the amino acid sequence of SEQ ID NO: 6; or
(b) HCDR1 comprising the amino acid sequence of SEQ ID NO: 101; (b) HCDR2 comprising the amino acid sequence of SEQ ID NO: 102; (c) HCDR3 comprising the amino acid sequence of SEQ ID NO: 103; with or without (d) LCDR1 comprising the amino acid sequence of SEQ ID NO: 104; (e) LCDR2 comprising the amino acid sequence of SEQ ID NO: 105; and (f) LCDR3 comprising the amino acid sequence of SEQ ID NO: 106; or
(c) HCDR1 comprising the amino acid sequence of SEQ ID NO: 201; (b) HCDR2 comprising the amino acid sequence of SEQ ID NO: 202; (c) HCDR3 comprising the amino acid sequence of SEQ ID NO: 203; with or without (d) LCDR1 comprising the amino acid sequence of SEQ ID NO: 204; (e) LCDR2 comprising the amino acid sequence of SEQ ID NO: 205; and (f) LCDR3 comprising the amino acid sequence of SEQ ID NO: 206; or
(d) HCDR1 comprising the amino acid sequence of SEQ ID NO: 301; (b) HCDR2 comprising the amino acid sequence of SEQ ID NO: 302; (c) HCDR3 comprising the amino acid sequence of SEQ ID NO: 303; with or without (d) LCDR1 comprising the amino acid sequence of SEQ ID NO: 304; (e) LCDR2 comprising the amino acid sequence of SEQ ID NO: 305; and (f) LCDR3 comprising the amino acid sequence of SEQ ID NO: 306; or
(e) HCDR1 comprising the amino acid sequence of SEQ ID NO: 401; (b) HCDR2 comprising the amino acid sequence of SEQ ID NO: 402; (c) HCDR3 comprising the amino acid sequence of SEQ ID NO: 403; with or without (d) LCDR1 comprising the amino acid sequence of SEQ ID NO: 404; (e) LCDR2 comprising the amino acid sequence of SEQ ID NO: 405; and (f) LCDR3 comprising the amino acid sequence of SEQ ID NO: 406; or
(f) HCDR1 comprising the amino acid sequence of SEQ ID NO: 501; (b) HCDR2 comprising the amino acid sequence of SEQ ID NO: 502; (c) HCDR3 comprising the amino acid sequence of SEQ ID NO: 503; with or without (d) LCDR1 comprising the amino acid sequence of SEQ ID NO: 504; (e) LCDR2 comprising the amino acid sequence of SEQ ID NO: 505; and (f) LCDR3 comprising the amino acid sequence of SEQ ID NO: 506; or
(g) HCDR1 comprising the amino acid sequence of SEQ ID NO: 601; (b) HCDR2 comprising the amino acid sequence of SEQ ID NO: 602; (c) HCDR3 comprising the amino acid sequence of SEQ ID NO: 603; with or without (d) LCDR1 comprising the amino acid sequence of SEQ ID NO: 604; (e) LCDR2 comprising the amino acid sequence of SEQ ID NO: 605; and (f) LCDR3 comprising the amino acid sequence of SEQ ID NO: 606; or
(h) HCDR1 comprising the amino acid sequence of SEQ ID NO: 701; (b) HCDR2 comprising the amino acid sequence of SEQ ID NO: 702; (c) HCDR3 comprising the amino acid sequence of SEQ ID NO: 703; with or without (d) LCDR1 comprising the amino acid sequence of SEQ ID NO: 704; (e) LCDR2 comprising the amino acid sequence of SEQ ID NO: 705; and (f) LCDR3 comprising the amino acid sequence of SEQ ID NO: 706; or
(i) HCDR1 comprising the amino acid sequence of SEQ ID NO: 801; (b) HCDR2 comprising the amino acid sequence of SEQ ID NO: 802; (c) HCDR3 comprising the amino acid sequence of SEQ ID NO: 803; with or without (d) LCDR1 comprising the amino acid sequence of SEQ ID NO: 804; (e) LCDR2 comprising the amino acid sequence of SEQ ID NO: 805; and (f) LCDR3 comprising the amino acid sequence of SEQ ID NO: 806; or
(j) (a) HCDR1 comprising the amino acid sequence of SEQ ID NO: 901; (b) HCDR2 comprising the amino acid sequence of SEQ ID NO: 902; (c) HCDR3 comprising the amino acid sequence of SEQ ID NO: 903; (d) LCDR1 comprising the amino acid sequence of SEQ ID NO: 904; (e) LCDR2 comprising the amino acid sequence of SEQ ID NO: 905; and (f) LCDR3 comprising the amino acid sequence of SEQ ID NO: 906; or
(k) (a) HCDR1 comprising the amino acid sequence of SEQ ID NO: 1001; (b) HCDR2 comprising the amino acid sequence of SEQ ID NO: 1002; (c) HCDR3 comprising the amino acid sequence of SEQ ID NO: 1003; (d) LCDR1 comprising the amino acid sequence of SEQ ID NO: 1004; (e) LCDR2 comprising the amino acid sequence of SEQ ID NO: 1005; and (f) LCDR3 comprising the amino acid sequence of SEQ ID NO: 1006; or
(l) (a) HCDR1 comprising the amino acid sequence of SEQ ID NO: 2001; (b) HCDR2 comprising the amino acid sequence of SEQ ID NO: 2002; (c) HCDR3 comprising the amino acid sequence of SEQ ID NO: 2003; (d) LCDR1 comprising the amino acid sequence of SEQ ID NO: 2004; (e) LCDR2 comprising the amino acid sequence of SEQ ID NO: 2005; and (f) LCDR3 comprising the amino acid sequence of SEQ ID NO: 2006; or
(m) (a) HCDR1 comprising the amino acid sequence of SEQ ID NO: 3001; (b) HCDR2 comprising the amino acid sequence of SEQ ID NO: 3002; (c) HCDR3 comprising the amino acid sequence of SEQ ID NO: 3003; (d) LCDR1 comprising the amino acid sequence of SEQ ID NO: 3004; (e) LCDR2 comprising the amino acid sequence of SEQ ID NO: 3005; and (f) LCDR3 comprising the amino acid sequence of SEQ ID NO: 3006; or
(n) (a) HCDR1 comprising the amino acid sequence of SEQ ID NO: 4001; (b) HCDR2 comprising the amino acid sequence of SEQ ID NO: 4002; (c) HCDR3 comprising the amino acid sequence of SEQ ID NO: 4003; (d) LCDR1 comprising the amino acid sequence of SEQ ID NO: 4004; (e) LCDR2 comprising the amino acid sequence of SEQ ID NO: 4005; and (f) LCDR3 comprising the amino acid sequence of SEQ ID NO: 4006.

In certain embodiments, a CD112R antibody comprises a VL comprising VL CDR1, CDR2 and CDR3 of any of the CD112R antibodies provided herein. In certain embodiments, a CD112R antibody comprises a VL comprising VL CDR1, CDR2 and CDR3 of any one of antibody clone numbers 2, 5, 44, 58, 10, 38, 15, 35, 46, 47, 32, 33, 34, or 36.

In some embodiments, a CD112R antibody may comprise:
(a) a VH comprising the amino acid sequence of the VH CDR1, CDR2 and CDR3 of antibody clone number 2 and a VL comprising the VL CDR1, CDR2 and CDR3 of antibody clone number 2; or
(b) a VH comprising the amino acid sequence of the VH CDR1, CDR2 and CDR3 of antibody clone number 5 and a VL comprising the VL CDR1, CDR2 and CDR3 of antibody clone number 5; or
(c) a VH comprising the amino acid sequence of the VH CDR1, CDR2 and CDR3 of antibody clone number 44 and a VL comprising the VL CDR1, CDR2 and CDR3 of antibody clone number 44; or
(d) a VH comprising the amino acid sequence of the VH CDR1, CDR2 and CDR3 of antibody clone number 58 and a VL comprising the VL CDR1, CDR2 and CDR3 of antibody clone number 58; or
(e) a VH comprising the amino acid sequence of the VH CDR1, CDR2 and CDR3 of antibody clone number 10 and a VL comprising the VL CDR1, CDR2 and CDR3 of antibody clone number 10; or
(f) a VH comprising the amino acid sequence of the VH CDR1, CDR2 and CDR3 of antibody clone number 38 and a VL comprising the VL CDR1, CDR2 and CDR3 of antibody clone number 38; or
(g) a VH comprising the amino acid sequence of the VH CDR1, CDR2 and CDR3 of antibody clone number 15 and a VL comprising the VL CDR1, CDR2 and CDR3 of antibody clone number 15; or
(h) a VH comprising the amino acid sequence of the VH CDR1, CDR2 and CDR3 of antibody clone number 35 and a VL comprising the VL CDR1, CDR2 and CDR3 of antibody clone number 35; or
(i) a VH comprising the amino acid sequence of the VH CDR1, CDR2 and CDR3 of antibody clone number 47 and a VL comprising the VL CDR1, CDR2 and CDR3 of antibody clone number 47; or
(j) a VH comprising the amino acid sequence of the VH CDR1, CDR2 and CDR3 of antibody clone number 46 and a VL comprising the VL CDR1, CDR2 and CDR3 of antibody clone number 46; or
(k) a VH comprising the amino acid sequence of the VH CDR1, CDR2 and CDR3 of antibody clone number 32 and a VL comprising the VL CDR1, CDR2 and CDR3 of antibody clone number 32; or
(l) a VH comprising the amino acid sequence of the VH CDR1, CDR2 and CDR3 of antibody clone number 33 and a VL comprising the VL CDR1, CDR2 and CDR3 of antibody clone number 33; or
(m) a VH comprising the amino acid sequence of the VH CDR1, CDR2 and CDR3 of antibody clone number 34 and a VL comprising the VL CDR1, CDR2 and CDR3 of antibody clone number 34; or
(n) a VH comprising the amino acid sequence of the VH CDR1, CDR2 and CDR3 of antibody clone number 36 and a VL comprising the VL CDR1, CDR2 and CDR3 of antibody clone number 36.

The Sequence Table below provides the heavy and light chain variable region sequences of certain disclosed antibodies.

In certain embodiments, a CD112R antibody comprises a VH comprising the amino acid sequence of the VH of any of the CD112R antibodies provided herein. In certain embodiments, a CD112R antibody comprises a VH comprising the amino acid sequence of the VH of any one of the antibody clone numbers 2, 5, 44, 58, 10, 38, 15, 35, 46, 47, 32, 33, 34, or 36.

In some embodiments, a CD112R antibody comprises the VH of any one of antibody clone numbers 2, 5, 44, 58, 10, 38, 15, 35, 46, 47, 32, 33, 34, or 36 but with 1, 2, 3, 4, or 5 amino acid substitutions outside the complementarity determining regions (CDRs), such as 1, 2, 3, 4, or 5 conservative substitutions outside the CDRs. In some embodiments, a CD112R antibody comprises the VH of any one of antibody clone numbers 2, 5, 44, 58, 10, 38, 15, 35, 46, 47, 32, 33, 34, or 36 but with 1, 2, 3, 4, or 5 reversion substitutions outside the complementarity determining regions (CDRs).

In some embodiments, a CD112R antibody comprises the VH of any one of antibody clone numbers 2, 5, 44, 58, 10, 38, 15, 35, 46, 47, 32, 33, 34, or 36 but with 1, 2, 3, 4, or 5 amino acid substitutions in the framework regions of the VH sequence, such as 1, 2, 3, 4, or 5 conservative substitutions. In some embodiments, a CD112R antibody comprises the VH of any one of antibody clone numbers 2, 5, 44, 58, 10, 38, 15, 35, 46, 47, 32, 33, 34, or 36 but with 1, 2, 3, 4, or 5 reversion substitutions in the framework regions of the VH sequence.

In some embodiments, a CD112R antibody comprises the VH and VL CDRs of any of the CD112R antibodies described herein, wherein each CDR comprises 0, 1, 2 or 3 amino acid additions, substitutions (e.g., conservative substitutions), or deletions.

In certain embodiments, a CD112R antibody comprises a VH CDR1, CDR2, and CDR3 comprising the amino acid sequences of the VH CDRs of any of the CD112R antibodies provided herein and comprises a VH that is at least 90%, at least 91%, at least 92%, at least 93%, at least 94%, at least 95%, at least 96%, at least 97%, at least 98%, or at least 99% identical to the VH of any of the CD112R antibodies provided herein. In certain embodiments, a CD112R antibody comprises a VH comprising an amino acid sequence that is at least 90%, at least 91%, at least 92%, at least 93%, at least 94%, at least 95%, at least 96%, at least 97%, at least 98%, or at least 99% identical to the amino acid sequence of the VH of any one of antibody clone numbers 2, 5, 44, 58, 10, 38, 15, 35, 46, 47, 32, 33, 34, or 36. In certain embodiments, the VH of the antibody differs from that of the VH sequences shown in the Sequence Table due to 1, 2, 3, 4, or 5 amino acid substitutions in the framework regions of the VH sequence, such as 1, 2, 3, 4, or 5 conservative substitutions. In certain embodiments, the VH of the antibody differs from that of the VH sequences shown in the Sequence Table due to 1, 2, 3, 4, or 5 reversion substitutions in the framework regions of the VH sequence.

In certain embodiments, a CD112R antibody comprises a VH consisting of the amino acid sequence of the VH of any of the CD112R antibodies provided herein. In certain embodiments, a CD112R antibody comprises a VH that consists of the amino acid sequence of the VH of any one of antibody clone numbers 2, 5, 44, 58, 10, 38, 15, 35, 46, 47, 32, 33, 34, or 36.

In certain embodiments, a CD112R antibody comprises a VL comprising the amino acid sequence of the VL of any of the CD112R antibodies provided herein. In certain embodiments, a CD112R antibody comprises a VL comprising the amino acid sequence of the VL of any one of antibody clone numbers 2, 5, 44, 58, 10, 38, 15, 35, 46, 47, 32, 33, 34, or 36. In certain embodiments, a CD112R antibody comprises a VL CDR1, CDR2, and CDR3 comprising the amino acid sequences of the VL CDRs of any of the CD112R antibodies provided herein and comprises a VL that is at least 90%, at least 91%, at least 92%, at least 93%, at least 94%, at least 95%, at least 96%, at least 97%, at least 98%, or at least 99% identical to the VL of any of the CD112R antibodies provided herein. In certain embodiments, a CD112R antibody comprises a VL comprising an amino acid sequence that is at least 90%, at least 91%, at least 92%, at least 93%, at least 94%, at least 95%, at least 96%, at least 97%, at least 98%, or at least 99% identical to the amino acid sequence of the VL of any one of antibody clone numbers 2, 5, 44, 58, 10, 38, 15, 35, 46, 47, 32, 33, 34, or 36. In certain embodiments, the VL of the antibody differs from that of the VL sequences shown in the Sequence Table due to 1, 2, 3, 4, or 5 amino acid substitutions in the framework regions of the VL sequence, such as 1, 2, 3, 4, or 5 conservative substitutions. In certain embodiments, the VL of the antibody differs from that of the VL sequences shown in the Sequence Table due to 1, 2, 3, 4, or 5 reversion substitutions.

In certain embodiments, a CD112R antibody comprises a VL consisting of the amino acid sequence of the VL of any of the CD112R antibodies provided herein. In certain embodiments, a CD112R antibody comprises a VL that consists of the amino acid sequence of the VL of any one of antibody clone numbers 2, 5, 44, 58, 10, 38, 15, 35, 46, 47, 32, 33, 34, or 36.

In certain embodiments, a CD112R antibody comprises a VH comprising the amino acid sequence of the VH of any of the CD112R antibodies provided herein and comprises a VL comprising the amino acid sequence of the VL of any of the same CD112R antibodies provided herein. In certain of these embodiments, a CD112R antibody comprises a VH comprising the amino acid sequence of the VH of any one of antibody clone numbers 2, 5, 44, 58, 10, 38, 15, 35, 46, 47, 32, 33, 34, or 36 and a VL comprising the amino acid sequence of the VL of any one of antibody clone numbers 2, 5, 44, 58, 10, 38, 15, 35, 46, 47, 32, 33, 34, or 36, optionally wherein the VH and VL are from the same antibody clone number.

In certain embodiments, the VH of the antibody is that of any one of antibody clone numbers 2, 5, 44, 58, 10, 38, 15, 35, 46, 47, 32, 33, 34, or 36, but with 1, 2, 3, 4, or 5 amino acid substitutions in the framework regions of the VH sequence, such as 1, 2, 3, 4, or 5 conservative substitutions, and the VL is that of any one of the same antibody from the list above. In certain embodiments, however, the VH of the antibody is that of any one of antibody clone numbers 2, 5, 44, 58, 10, 38, 15, 35, 46, 47, 32, 33, 34, or 36, but with 1, 2, 3, 4, or 5 substitutions in the framework regions of the VH sequence.

In certain embodiments, a CD112R antibody comprises a VH and a VL comprising the amino acid sequences of the VH and VL of any one of antibody clone numbers 2, 5, 44, 58, 10, 38, 15, 35, 46, 47, 32, 33, 34, or 36.

In certain embodiments, a CD112R antibody comprises a VH CDR1, CDR2, and CDR3 comprising the amino acid sequences of the VH CDRs of any of the CD112R antibodies provided herein as well as a VL CDR1, CDR2, and CDR3 comprising the amino acid sequences of the VL CDRs of any of the CD112R antibodies provided herein, and also comprises a VH and a VL that are each at least 90%, at least 91%, at least 92%, at least 93%, at least 94%, at least 95%, at least 96%, at least 97%, at least 98%, or at least 99% identical to the corresponding VH and VL of any of the CD112R antibodies provided herein. In certain embodiments, the VH and the VL of the antibody differ from the VH and VL sequences shown in the Sequence Table due to 1, 2, 3, 4, or 5 amino acid substitutions in the framework regions of the sequences, such as 1, 2, 3, 4, or 5 conservative substitutions, or such as 1, 2, 3, 4 or 5 reversion substitutions.

In certain embodiments, a CD112R antibody comprises a VH and a VL consisting of the amino acid sequence of the VH and VL of any of the CD112R antibodies provided herein. In certain embodiments, a CD112R antibody comprises a VH and a VL that each consist of the amino acid sequences of the VH and VL of any one of antibody clone numbers 2, 5, 44, 58, 10, 38, 15, 35, 46, 47, 32, 33, 34, and 36.

A CD112R antibody may comprise:
(a) a VH comprising the amino acid sequence of the VH of antibody clone number 2 and a VL comprising the amino acid sequence of the VL of antibody clone number 2; or
(b) a VH comprising the amino acid sequence of the VH of antibody clone number 5 and a VL comprising the amino acid sequence of the VL of antibody clone number 5; or
(c) a VH comprising the amino acid sequence of the VH of antibody clone number 44 and a VL comprising the amino acid sequence of the VL of antibody clone number 44; or
(d) a VH comprising the amino acid sequence of the VH of antibody clone number 58 and a VL comprising the amino acid sequence of the VL of antibody clone number 58; or
(e) a VH comprising the amino acid sequence of the VH of antibody clone number 10 and a VL comprising the amino acid sequence of the VL of antibody clone number 10; or
(f) a VH comprising the amino acid sequence of the VH of antibody clone number 38 and a VL comprising the amino acid sequence of the VL of antibody clone number 38; or
(g) a VH comprising the amino acid sequence of the VH of antibody clone number 15 and a VL comprising the amino acid sequence of the VL of antibody clone number 15; or
(h) a VH comprising the amino acid sequence of the VH of antibody clone number 35 and a VL comprising the amino acid sequence of the VL of antibody clone number 35; or
(i) a VH comprising the amino acid sequence of the VH of antibody clone number 47 and a VL comprising the amino acid sequence of the VL of antibody clone number 47;
(j) a VH comprising the amino acid sequence of the VH of antibody clone number 46 and a VL comprising the amino acid sequence of the VL of antibody clone number 46;
(k) a VH comprising the amino acid sequence of the VH of antibody clone number 32 and a VL comprising the amino acid sequence of the VL of antibody clone number 32; or
(l) a VH comprising the amino acid sequence of the VH of antibody clone number 33 and a VL comprising the amino acid sequence of the VL of antibody clone number 33; or
(m) a VH comprising the amino acid sequence of the VH of antibody clone number 34 and a VL comprising the amino acid sequence of the VL of antibody clone number 34; or
(n) a VH comprising the amino acid sequence of the VH of antibody clone number 36 and a VL comprising the amino acid sequence of the VL of antibody clone number 36; or.

A CD112R antibody may comprise:
(a) a VH comprising the VH CDRs of the VH of antibody clone number 2, and a VL comprising the VL CDRs of antibody clone number 2, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 2; or
(b) a VH comprising the VH CDRs of the VH of antibody clone number 5, and a VL comprising the VL CDRs of antibody clone number 5, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 5; or
(c) a VH comprising the VH CDRs of the VH of antibody clone number 44, and a VL comprising the VL CDRs of antibody clone number 44, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 44; or
(d) a VH comprising the VH CDRs of the VH of antibody clone number 58, and a VL comprising the VL CDRs of antibody clone number 58, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 58; or
(e) a VH comprising the VH CDRs of the VH of antibody clone number 10, and a VL comprising the VL CDRs of antibody clone number 10, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 10; or
(f) a VH comprising the VH CDRs of the VH of antibody clone number 38, and a VL comprising the VL CDRs of antibody clone number 38, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 38; or
(g) a VH comprising the VH CDRs of the VH of antibody clone number 15, and a VL comprising the VL CDRs of antibody clone number 15, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 15; or
(h) a VH comprising the VH CDRs of the VH of antibody clone number 35, and a VL comprising the VL CDRs of antibody clone number 35, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 35; or
(i) a VH comprising the VH CDRs of the VH of antibody clone number 47, and a VL comprising the VL CDRs of antibody clone number 47, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 47; or
(j) a VH comprising the VH CDRs of the VH of antibody clone number 46, and a VL comprising the VL CDRs of antibody clone number 46, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 46; or
(k) a VH comprising the VH CDRs of the VH of antibody clone number 32, and a VL comprising the VL CDRs of antibody clone number 32, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 32; or
(l) a VH comprising the VH CDRs of the VH of antibody clone number 33, and a VL comprising the VL CDRs of antibody clone number 33, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 33; or
(m) a VH comprising the VH CDRs of the VH of antibody clone number 34, and a VL comprising the VL CDRs of antibody clone number 34, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 34; or
(n) a VH comprising the VH CDRs of the VH of antibody clone number 36, and a VL comprising the VL CDRs of antibody clone number 36, and VH and VL amino acid sequences that are at least 90%, at least 91%, at least 92%, at least 93%, at least 94%, at least 95%, at least 96%, at least 97%, at least 98%, or at least 99% identical to the VH and VL of antibody clone number 36.

In some of the above embodiments, the VH and/or VL may differ from the sequence of each of the species by the presence of 1, 2, 3, 4, or 5 amino acid substitutions, such as 1, 2, 3, 4, or 5 conservative substitutions. In some embodiments, the VH may comprise 1, 2, 3, 4, or 5 reversion substitutions.

A CD112R antibody may comprise:
(a) a VH consisting of the amino acid sequence of the VH of antibody clone number 2 and a VL consisting of the VL of antibody clone number 2; or
(b) a VH consisting of the amino acid sequence of the VH of antibody clone number 5 and a VL consisting of the VL of antibody clone number 5; or
(c) a VH consisting of the amino acid sequence of the VH of antibody clone number 44 and a VL consisting of the VL of antibody clone number 44; or
(d) a VH consisting of the amino acid sequence of the VH of antibody clone number 58 and a VL consisting of the VL of antibody clone number 58; or
(e) a VH consisting of the amino acid sequence of the VH of antibody clone number 10 and a VL consisting of the VL of antibody clone number 10; or
(f) a VH consisting of the amino acid sequence of the VH of antibody clone number 38 and a VL consisting of the VL of antibody clone number 38; or
(g) a VH consisting of the amino acid sequence of the VH of antibody clone number 15 and a VL consisting of the VL of antibody clone number 15; or
(h) a VH consisting of the amino acid sequence of the VH of antibody clone number 35 and a VL consisting of the VL of antibody clone number 35; or
(i) a VH consisting of the amino acid sequence of the VH of antibody clone number 47 and a VL consisting of the VL of antibody clone number 47; or
(j) a VH consisting of the amino acid sequence of the VH of antibody clone number 46 and a VL consisting of the VL of antibody clone number 46; or
(k) a VH consisting of the amino acid sequence of the VH of antibody clone number 32 and a VL consisting of the VL of antibody clone number 32; or
(l) a VH consisting of the amino acid sequence of the VH of antibody clone number 33 and a VL consisting of the VL of antibody clone number 33; or
(m) a VH consisting of the amino acid sequence of the VH of antibody clone number 34 and a VL consisting of the VL of antibody clone number 34; or
(n) a VH consisting of the amino acid sequence of the VH of antibody clone number 36 and a VL consisting of the VL of antibody clone number 36.

In certain embodiments, a CD112R antibody comprises any of the variable regions and/or variable region CDRs 1-3 of the antibodies described above and elsewhere herein, such as in the Sequence Table.

In some embodiments, the CD112R antibody is an IgG antibody, such as IgG1, IgG2, IgG3 or IgG4 antibody or a modified form thereof as described in the section below. In some embodiments, the constant region has effector function, and in some embodiments, the constant region is effectorless.

In certain embodiments, a CD112R antibody comprises a heavy chain (HC) comprising the amino acid sequence of the heavy chain of any of the CD112R antibodies provided herein. In certain embodiments, a CD112R antibody comprises a heavy chain comprising the amino acid sequence of the heavy chain of any one of antibody clone numbers 2, 5, 44, 58, 10, 38, 15, 35, 46, 47, 32, 33, 34, or 36.

In some embodiments, a CD112R antibody may comprise:
(a) a heavy chain comprising the amino acid sequence of the heavy chain of antibody clone number 2 and a light chain comprising the light chain amino acid sequence of antibody clone number 2; or
(b) a heavy chain comprising the amino acid sequence of the heavy chain of antibody clone number 5 and a light chain comprising the light chain amino acid sequence of antibody clone number 5; or
(c) a heavy chain comprising the amino acid sequence of the heavy chain of antibody clone number 44 and a light chain comprising the light chain amino acid sequence of antibody clone number 44; or
(d) a heavy chain comprising the amino acid sequence of the heavy chain of antibody clone number 58 and a light chain comprising the light chain amino acid sequence of antibody clone number 58; or
(e) a heavy chain comprising the amino acid sequence of the heavy chain of antibody clone number 10 and a light chain comprising the light chain amino acid sequence of antibody clone number 10; or
(f) a heavy chain comprising the amino acid sequence of the heavy chain of antibody clone number 38 and a light chain comprising the light chain amino acid sequence of antibody clone number 38; or
(g) a heavy chain comprising the amino acid sequence of the heavy chain of antibody clone number 15 and a light chain comprising the light chain amino acid sequence of antibody clone number 15; or
(h) a heavy chain comprising the amino acid sequence of the heavy chain of antibody clone number 35 and a light chain comprising the light chain amino acid sequence of antibody clone number 35; or
(i) a heavy chain comprising the amino acid sequence of the heavy chain of antibody clone number 47 and a light chain comprising the light chain amino acid sequence of antibody clone number 47; or
(j) a heavy chain comprising the amino acid sequence of the heavy chain of antibody clone number 46 and a light chain comprising the light chain amino acid sequence of antibody clone number 46; or
(k) a heavy chain comprising the amino acid sequence of the heavy chain of antibody clone number 32 and a light chain comprising the light chain amino acid sequence of antibody clone number 32; or
(l) a heavy chain comprising the amino acid sequence of the heavy chain of antibody clone number 33 and a light chain comprising the light chain amino acid sequence of antibody clone number 33; or
(m) a heavy chain comprising the amino acid sequence of the heavy chain of antibody clone number 34 and a light chain comprising the light chain amino acid sequence of antibody clone number 34; or
(n) a heavy chain comprising the amino acid sequence of the heavy chain of antibody clone number 36 and a light chain comprising the light chain amino acid sequence of antibody clone number 36.

A CD112R antibody may comprise:
(a) a heavy chain (HC) comprising the HC CDRs of the HC of antibody clone number 2 and a light chain (LC) comprising the LC CDRs of antibody clone number 2 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 2, respectively; or
(b) a HC comprising the HC CDRs of the HC of antibody clone number 5, and a light chain (LC) comprising the LC CDRs of antibody clone number 5 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 5, respectively; or
(c) a HC comprising the HC CDRs of the HC of antibody clone number 44, and a light chain (LC) comprising the LC CDRs of antibody clone number 44 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 44, respectively; or
(d) a HC comprising the HC CDRs of the HC of antibody clone number 58, and a light chain (LC) comprising the LC CDRs of antibody clone number 58 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 58, respectively; or
(e) a HC comprising the HC CDRs of the HC of antibody clone number 10, and a light chain (LC) comprising the LC CDRs of antibody clone number 10 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 10, respectively; or
(f) a HC comprising the HC CDRs of the HC of antibody clone number 38, and a light chain (LC) comprising the LC CDRs of antibody clone number 38 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 38, respectively; or
(g) a HC comprising the HC CDRs of the HC of antibody clone number 15, and a light chain (LC) comprising the LC CDRs of antibody clone number 15 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 15, respectively; or
(h) a HC comprising the HC CDRs of the HC of antibody clone number 35, and a light chain (LC) comprising the LC CDRs of antibody clone number 35 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 35, respectively; or
(i) a HC comprising the HC CDRs of the HC of antibody clone number 47, and a light chain (LC) comprising the LC CDRs of antibody clone number 47 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 47, respectively; or
(j) a HC comprising the HC CDRs of the HC of antibody clone number 46, and a light chain (LC) comprising the LC CDRs of antibody clone number 46 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 46, respectively; or
(k) a HC comprising the HC CDRs of the HC of antibody clone number 32, and a LC comprising the LC CDRs of antibody clone number 32 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 32, respectively; or
(l) a HC comprising the HC CDRs of the HC of antibody clone number 33, and a light chain (LC) comprising the LC CDRs of antibody clone number 33 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 33, respectively; or
(m) a HC comprising the HC CDRs of the HC of antibody clone number 34, and a light chain (LC) comprising the LC CDRs of antibody clone number 34 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 34, respectively; or
(n) a HC comprising the HC CDRs of the HC of antibody clone number 36, and a light chain (LC) comprising the LC CDRs of antibody clone number 36 and HC and LC amino acid sequences that are at least 90%, at least 91%, at least 92%, at least 93%, at least 94%, at least 95%, at least 96%, at least 97%, at least 98%, or at least 99% identical to the HC and LC of antibody clone number 36, respectively.

In some of the above embodiments, the HC and/or LC may differ from the sequence of each of the species by the presence of 1, 2, 3, 4, or 5 amino acid substitutions, such as 1, 2, 3, 4, or 5 conservative substitutions. In some of the above embodiments, the HC and/or LC may differ from the sequence of each of the species by the presence of 1, 2, 3, 4, or 5 amino acid substitutions, such as 1, 2, 3, 4, or 5 reversion substitutions.

1. Exemplary Class of Antibodies with Shared Structural and Functional Features

In some embodiments, anti-CD112R antibodies are provided. In certain embodiments, the anti-CD112 antibodies share certain structural and/or functional features. In some embodiments, the class of antibodies includes a parent antibody and affinity matured variants thereof. One exemplary class of antibodies includes, but is not limited to, parent clone 32, and affinity matured variants thereof. In some embodiments, the affinity matured variants comprise antibodies 33, 34, 35, and 36. In some embodiments, the affinity matured variants comprise antibodies with conservative substitutions as compared to antibodies 32, 33, 34, 35 and 36.

a. Structural Features of Exemplary Class of Antibodies

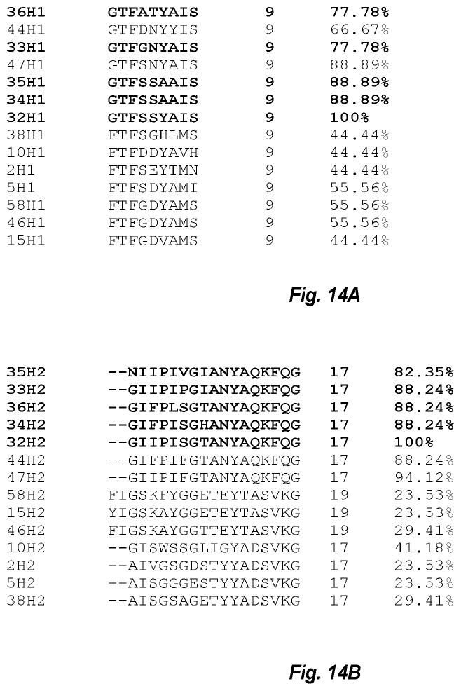

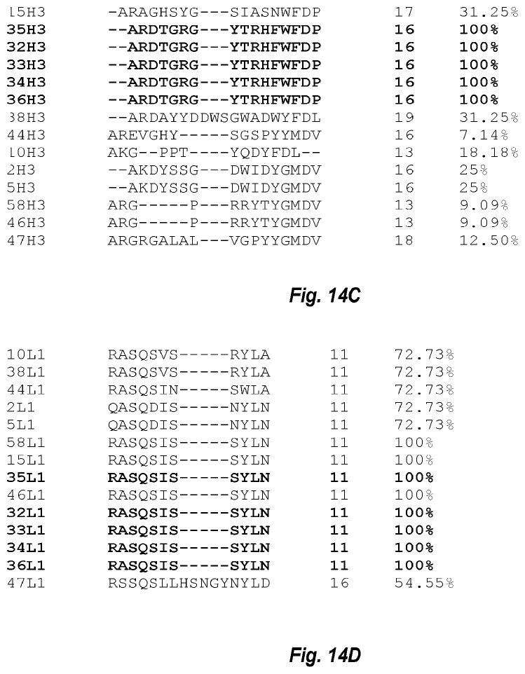

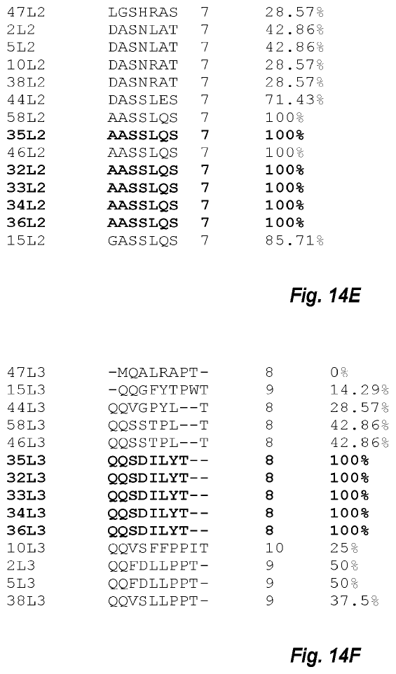

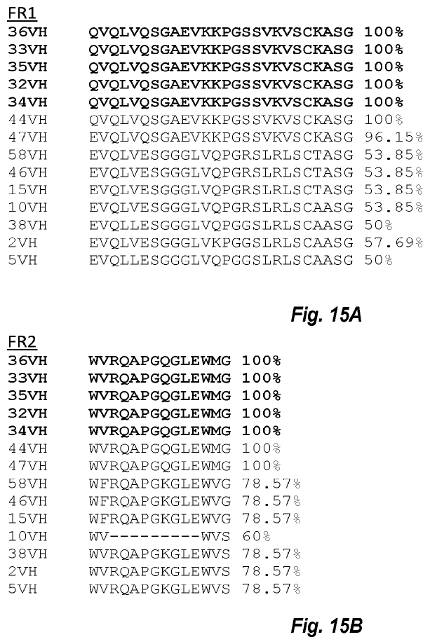

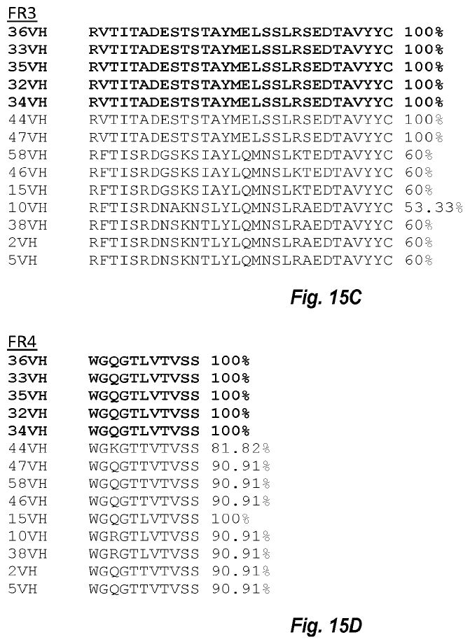

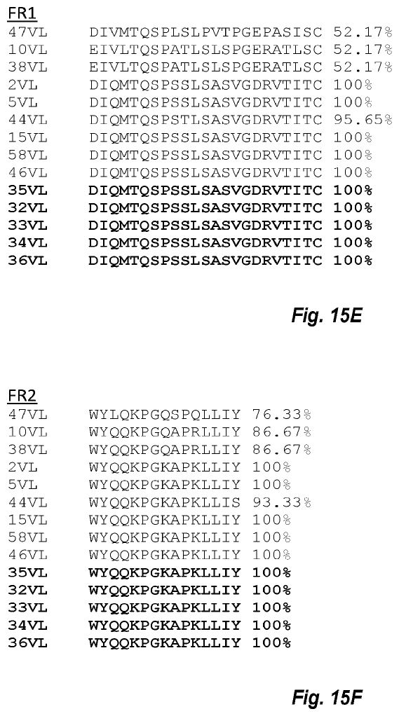

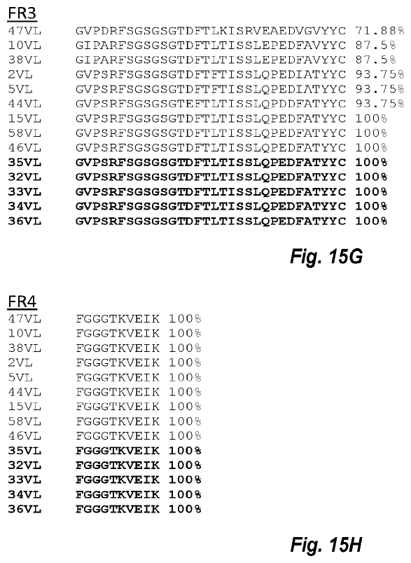

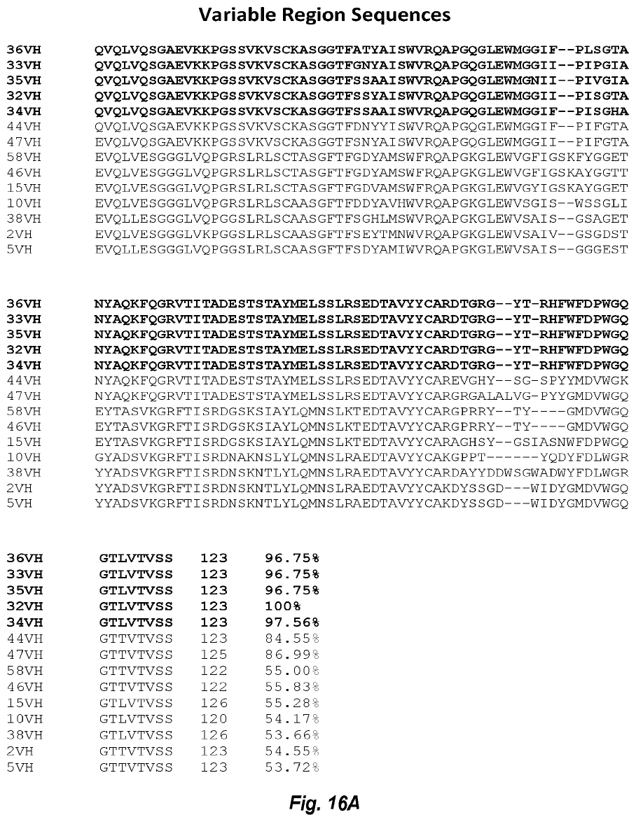

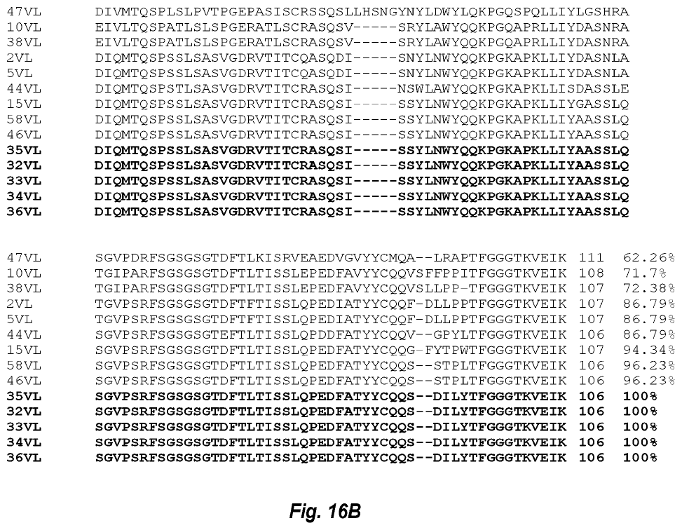

In some embodiments, the anti-CD112R antibodies share structural features, such as, for example, those shown in FIGS. 14 and 15. When a “class” or “members of a class” of antibodies is described herein, it is to be understood that embodiments describing a single anti-CD112R antibody or multiple anti-CD112R antibodies is encompassed/envisioned. In some embodiments, the anti-CD112R antibodies comprise identical HCDR3s. In some embodiments, the anti-CD112R antibodies comprise identical LCDR1s. In some embodiments, the anti-CD112R antibodies comprise identical LCDR2s. In some embodiments, the anti-CD112R antibodies comprise identical LCDR3s. In some embodiments, the anti-CD112R antibodies comprise identical HCDR3s and identical LCDR1s, LCDR2s, and/or LCDR3s. In some embodiments, the anti-CD112R antibodies comprise HCDR3 comprising the amino acid sequence of SEQ ID NO: 1003, and/or LCDR1 comprising the amino acid sequence of SEQ ID NO: 1004, and/or LCDR2 comprising the amino acid sequence of SEQ ID NO: 1005, and/or LCDR3 comprising the amino acid sequence of SEQ ID NO: 1006.

In some embodiments, each member of the class of antibodies comprises heavy chain framework regions comprising the amino acid sequences of SEQ ID NOs: 1007, 1008, 1009, and 1010. In some embodiments, each member of the class of antibodies comprises a heavy chain variable region amino acid sequence that is at least 90%, at least 91%, at least 92%, or at least 93% identical to the amino acid sequence of SEQ ID NO: 1012, wherein any and all of the sequence variation relative to SEQ ID NO: 1012 is in HCDR1 and/or HCDR2. In some embodiments, each member of the class of antibodies comprises light chain framework regions comprising the amino acid sequences of SEQ ID NOs: 1013, 1014, 1015, and 1016. In some embodiments, each member of the class of antibodies comprises the light chain variable region amino acid sequence of SEQ ID NO: 1018.

Members of this exemplary class of antibodies may comprise some variation in the amino acid sequences of HCDR1 and HCDR2. In some embodiments, each member of the class of antibodies comprise HCDR1 comprising the amino acid sequence of SEQ ID NO: 1001 or the amino acid sequence of SEQ ID NO: 1001 with 1, 2, or 3 amino acid changes to positions 4, 5, and/or 6 of SEQ ID NO: 1001. In some embodiments, the class of antibodies comprise HCDR1 comprising the amino acid sequence of SEQ ID NO: 1001 with 1, 2, or 3 amino acid changes to positions that vary between the amino acids of SEQ ID NO: 1001, 2001, 3001, 4001, and 701 as shown in FIG. 14. In some embodiments, one or more of the 1, 2, or 3 amino acid changes are not conservative substitutions. In some embodiments, one or more of the 1, 2, or 3 amino acid changes are conservative substitutions. In certain embodiments, a member of the class of antibodies comprises HCDR1 comprising the amino acid sequence of SEQ ID NO: 2001, 3001, 701, or 4001.

In some embodiments, each member of the class of antibodies comprise HCDR2 comprising the amino acid sequence of SEQ ID NO: 1002 or the amino acid sequence of SEQ ID NO: 1002 with 1, 2, 3, 4, or 5 amino acid changes to positions 1, 3, 5, 6, and/or 8 of SEQ ID NO: 1002. In some embodiments, the class of antibodies comprise HCDR2 comprising the amino acid sequence of SEQ ID NO: 1002 with 1, 2, 3, 4, or 5 amino acid changes to positions that vary between the amino acids of SEQ ID NO: 1002, 2002, 3002, 4002, and 702 as shown in FIG. 14. In some embodiments, one or more of the 1, 2, 3, 4, or 5 amino acid changes are not conservative substitutions. In some embodiments, one or more of the 1, 2, 3, 4, or 5 amino acid changes are conservative substitutions. In certain embodiments, a member of the class of antibodies comprises HCDR2 comprising the amino acid sequence of SEQ ID NO: 2002, 3002, 702, or 4002.

In some embodiments, each member of the class of antibodies comprise identical heavy chain and light chain framework regions.

b. Functional Features of Exemplary Class of Antibodies

In some embodiments, anti-CD112R antibodies are provided, wherein the antibodies share a special technical effect of binding to human CD112R, blocking the interaction of human CD112R to CD112, and failing to block the interaction of mouse CD112R to CD112. In some embodiments, each member of a class of antibodies binds to human CD112R and blocks the binding interaction between human CD112 and human CD112R. In some embodiments, each member of the class of antibodies does not block the binding interaction between mouse CD112 and mouse CD112R. Although the members of the class of antibodies do not block the interaction between mouse CD112 and mouse CD112R, the members of the class of antibodies either partially inhibits the binding interaction between mouse CD112 and mouse CD112R or does not inhibit the binding interaction between mouse CD112 and mouse CD112R. In some such embodiments, no member of the class of antibodies inhibits the interaction between mouse CD112R and mouse CD112 by more than 50%. In some embodiments, each member of the class of antibodies exhibits at least some binding to soluble mouse CD112R. In some embodiments, the antibody is fully human or humanized. In some embodiments, each member of the class of antibodies binds the same epitope on human CD112R.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E, coli or phage), as described herein.

### 3. Multispecific Antibodies

In certain embodiments, an antibody provided herein is a multispecific antibody, e.g, a bispecific antibody. Multispecific antibodies are monoclonal antibodies that have binding specificities for at least two different sites. In certain embodiments, one of the binding specificities is for CD112R and the other is for any other antigen. In certain embodiments, one of the binding specificities is for CD112R and the other is for selected independently from one (in the case of bispecific) or more (in the case of multispecific) of PD-1, PD-L1, CTLA-4, Lag-3, TIM-3, TIGIT, CD96, PVRL1, PVRL2, PVRL3, PVRL4, CD155, STING, CD47, CD39, and IL-27. In certain embodiments, bispecific antibodies may bind to two different epitopes of CD112R. Bispecific antibodies may also be used to localize cytotoxic agents to cells which express CD112R. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)), WO 93/08829, and Traunecker et al., EMBO J. 10:3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147:60 (1991).

Engineered antibodies with three or more functional antigen binding sites, including “Octopus antibodies,” are also included herein (see, e.g. US 2006/0025576A1).

The antibody or fragment herein also includes a “Dual Acting Fantibody” or “DAF” comprising an antigen binding site that binds to CD112R as well as another, different antigen (see, US 2008/0069820, for example).

### 4. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### 5. Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 as are “exemplary substitutions. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al, in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### 6. Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g, complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y, et al., Biotechnol. Bioeng., 94 (4): 680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### 7. Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g, a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I, et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M, et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S, et al., Blood 101:1045-1052 (2003); and Cragg. M. S, and M. J. Glennie, Blood 103:2738-2743 (2004)). FcRn binding and in vivo clearance/half life determinations can also be performed using methods known in the art (see, e.g., Petkova, S. B, et al., Int'l. Immunol. 18 (12): 1759-1769 (2006)).

Antibodies with reduced effector function include those with substitution of one or more of Fc region residues 238, 265, 269, 270, 297, 327 and 329 (U.S. U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (e.g., U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some embodiments, an antibody is provided according to the Table of Sequences, wherein the isotype is human IgG1. In some embodiments, an antibody is provided according to the Table of Sequences, wherein the isotype is human IgG4. In some embodiments, an antibody is provided according to the Table of Sequences, wherein the isotype is human IgG4, wherein there is a single mutation at serine 228 to proline (S228P).

### 8. Cysteine Engineered Antibody Variants

In certain embodiments, it may be desirable to create cysteine engineered antibodies, e.g., “thioMantibodies,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### 9. Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods

Antibodies may be produced using recombinant methods and compositions, e.g., as described in U.S. Pat. No. 4,816,567. In some embodiments, isolated nucleic acid encoding an anti-CD112R antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In some embodiments, the host cell is eukaryotic, e.g, a Chinese Hamster Ovary (CHO) cell or lymphoid cell (e.g., Y0, NS0, Sp20 cell). In some embodiments, a method of making an anti-CD112R antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD112R antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology. Vol. 248 (B. K. C. Lo, ed., Humana Press, Totowa, NJ, 2003), pp. 245-254, describing expression of antibody fragments in E, coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology. Vol. 248 (B. K. C. Lo, ed., Humana Press, Totowa, NJ), pp. 255-268 (2003).

### C. Immunoconjugates

The invention also provides immunoconjugates comprising an anti-CD112R antibody herein conjugated to one or more other therapeutic agents or radioactive isotopes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody may be made using a variety of bifunctional protein coupling agents such as N-succinimidyl-3-(2-pyridyldithio) propionate (SPDP), succinimidyl-4-(N-maleimidomethyl) cyclohexane-1-carboxy late (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### D. Pharmaceutical Formulations and Compositions

Pharmaceutical formulations or compositions of an anti-CD112R antibody as described herein are prepared by mixing such antibody having the desired degree of purity with one or more optional pharmaceutically acceptable carriers, diluents, and/or excipients (Remington's Pharmaceutical Sciences 16th edition, Osol. A. Ed. (1980))), in the form of lyophilized formulations or aqueous solutions. Pharmaceutically acceptable carriers, diluents, and excipients are generally nontoxic to recipients at the dosages and concentrations employed, and include, but are not limited to: sterile water, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin. gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation or composition herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxy methylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations or compositions to be used for in vivo administration are generally sterile. Sterility may be readily accomplished, e.g., by filtration through sterile filtration membranes.

### E. Therapeutic Methods

Any of the anti-CD112R antibodies provided herein may be used in therapeutic methods. Throughout, where an “antibody” is discussed, it should also be appreciated that a composition comprising the antibody is also encompassed.

In one aspect, an anti-CD112R antibody for use as a medicament is provided. In some embodiments, an anti-CD112R antibody for use in enhancing, increasing and/or sustaining an anti-tumor immune response in a subject having a tumor is provided. In some embodiments, the tumor is cancerous. In some embodiments, an anti-CD112R antibody for use in treating cancer is provided. In some embodiments, an anti-CD112R antibody for use in enhancing CD226 interactions with CD112 is provided.

In a further aspect, the invention provides for the use of an anti-CD112R antibody in the manufacture or preparation of a medicament. In some embodiments, the medicament is for use in enhancing, increasing and/or sustaining an anti-tumor immune response in a subject having a tumor. In some embodiments, the tumor is cancerous. In some embodiments, the medicament is for treating cancer. In some embodiments, the medicament is for enhancing CD226 interactions with CD112.

In further aspects, the invention provides methods for treating diseases and/or disorders where blocking CD112R are desired. In some embodiments, methods for enhancing, increasing and/or sustaining an anti-tumor immune response in a subject having a tumor are provided comprising administering an anti-CD112R antibody as described herein. In some embodiments, the tumor is cancerous. In some embodiments, methods for treating cancer in a subject having cancer are provided comprising administering an anti-CD112R antibody as described herein. In some embodiments, methods for enhancing CD226 interactions with CD112 in a subject, optionally having cancer, are provided comprising administering an anti-CD112R antibody as described herein.

In some aspects, the invention provides a method for alleviating one or more symptoms of a CD112R protein associated disease or disorder; or an anti-CD112R antibody or a medicament comprising anti-CD112R antibody for alleviating one or more symptoms of a CD112R protein associated disease or disorder (such as any of the diseases or disorders described herein, for example, cancer). In some aspects, the invention provides a method for reducing the number of symptoms or the severity of one or more symptoms of a CD112R protein associated disease or disorder; or an anti-CD112R antibody or a medicament comprising anti-CD112R antibody for reducing the number of symptoms or the severity of one or more symptoms of a CD112R protein associated disease or disorder (such as any of the diseases or disorders described herein, for example, cancer). In a particular embodiment, the symptom of a CD112R protein associated disease or disorder is a tumor, and a reduction is a reduction in size of a tumor, the failure of the tumor to grow; or the elimination of the tumor.

The antibodies described herein may be used, for example, for treating cancer. In some embodiments, methods for treating cancer are provided, comprising administering an effective amount of an antibody described herein to a subject. In some embodiments, the antibodies may trigger or enhance an immune response in the subject, such as an antigen-specific immune response. In some embodiments, the antibodies may stimulate T cell activity. In some embodiments, the antibodies may inhibit the growth of at least one tumor in the subject.

Provided herein are methods for treating a subject having cancer, comprising administering to the subject a therapeutically effective amount of a CD112R antibody described herein, such that the subject is treated. A CD112R antibody can be used alone. Alternatively, a CD112R antibody can be used in conjunction with another agent, as described further below.

Cancers can be cancers with solid tumors or blood malignancies (e.g., liquid tumors).

Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma. Kaposi's sarcoma, epidermoid cancer, squamous cell cancer. T cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages. i.e., the myeloid cell line (which produces granulocytes, erythrocytes, thrombocytes, macrophages and mast cells) or lymphoid cell line (which produces B. T. NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (MI), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL). B cell hematologic malignancy, e.g., B cell lymphomas. T cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T cell lymphoma/leukemia, mantle cell lymphoma, angio immunoblastic T cell lymphoma, angiocentric lymphoma, intestinal T cell lymphoma, primary mediastinal B-cell lymphoma, precursor T-lymphoblastic lymphoma. T-lymphoblastic; and lymphoma/leukaemia (T-Lbly/T-ALL), peripheral T cell lymphoma, lymphoblastic lymphoma, post-transplantation lymphoproliferative disorder, true histiocytic lymphoma, primary central nervous system lymphoma, primary effusion lymphoma. B cell lymphoma, lymphoblastic lymphoma (LBL), hematopoietic tumors of lymphoid lineage, acute lymphoblastic leukemia, diffuse large B-cell lymphoma. Burkitt's lymphoma, follicular lymphoma, diffuse histiocytic lymphoma (DHL), immunoblastic large cell lymphoma, precursor B-lymphoblastic lymphoma, cutaneous T cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T cell and B cell tumors, including but not limited to T cell disorders such as T-prolymphocytic leukemia (T-PLL), including of the small cell and cerebriform cell type: large granular lymphocyte leukemia (LGL) of the T cell type; a/d T-NHL hepatosplenic lymphoma; peripheral/post-thymic T cell lymphoma (pleomorphic and immunoblastic subtypes); angiocentric (nasal) T cell lymphoma; cancer of the head or neck, renal cancer, rectal cancer, cancer of the thyroid gland; acute myeloid lymphoma, as well as any combinations of said cancers. The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

In certain embodiments, an antibody described herein is administered to subjects having a cancer that has exhibited an inadequate response to, or progressed on, a prior treatment, e.g., a prior treatment with an immuno-oncology or immunotherapy drug. In some embodiments, the cancer is refractory or resistant to a prior treatment, either intrinsically refractory or resistant (e.g., refractory to a PD-1 pathway antagonist), or a resistance or refractory state is acquired. For example, an antibody described herein may be administered to subjects who are not responsive or not sufficiently responsive to a first therapy or who have disease progression following treatment, e.g., anti-PD-1 pathway antagonist treatment, either alone or in combination with another therapy (e.g., with an anti-PD-1 pathway antagonist therapy). In other embodiments, an antibody described herein is administered to subjects who have not previously received (i.e., been treated with) an immuno-oncology agent, e.g., a PD-1 pathway antagonist.

### F. Combinations

Antibodies of the invention can be used either alone or in combination with other agents in a therapy. For instance, an antibody of the invention may be co-administered with at least one additional therapeutic agent (e.g., further comprising administering a second therapy).

In some embodiments, targeting an additional independent inhibitory pathway or combinations thereof has the potential to lead to further enhanced immune cell activation beyond monotherapy.

In some embodiments, the additional therapeutic agent or second agent is a chemotherapeutic agent, an opsonizing agent, a regulatory T cell (“Treg”) depleting agent, an antagonist of a target other than CD112R, or an agonist of a target other than CD112R. In certain embodiments, the second agent is a chemotherapeutic agent described herein or any known chemotherapeutic agent. In some embodiments, the second agent is an opsonizing agent, wherein the opsonizing agent is an antibody other than an anti-CD112R antibody that targets cancer or tumor cells. In some embodiments, the second agent is a Treg depleting agent described herein or any known Treg depleting agent. In some embodiments, the second agent is an antagonist of a target other than CD112R. In some embodiments, the second agent is an agonist of a target other than CD112R.

In some instances, the second agent targets an independent inhibitory pathway, such as, for example, a pathway involving PD-1, PD-L1, CTLA-4, Lag-3 or TIM-3. In some embodiments, the second agent antagonizes one or more of PD-1, PD-L1, CTLA-4, Lag-3 and TIM-3. Suitable antagonists for use in the combination therapy described herein, include, without limitation, ligands, antibodies (e.g., monoclonal antibodies and bispecific antibodies), and multivalent agents. In one embodiment, the antagonist is a fusion protein, e.g., an Fc fusion protein, such as AMP-244. In some embodiments, the PD-1 antagonist is an anti-PD-1 or anti-PD-L1 antibody.

An exemplary anti-PD-1 antibody is nivolumab (BMS-936558) or an antibody that comprises the CDRs or variable regions of one of antibodies 17D8, 2D3, 4H1, 5C4, 7D3, 5F4 and 4A11 described in WO 2006/121168. In certain embodiments, an anti-PD-1 antibody is MK-3475 (Lambrolizumab) described in WO2012/145493; AMP-514 described in WO 2012/145493; or PDR001. Further known PD-1 antibodies and other PD-1 inhibitors include those described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368. Any of the anti-PD-1 antibodies disclosed in WO2013/173223 can also be used. An anti-PD-1 antibody that competes for binding with, and/or binds to the same epitope on PD-1 as, as one of these antibodies can also be used in combination treatments.

In some embodiments, the anti-PD-L1 antibody useful for the combination therapy is BMS-936559 (referred to as 12A4 in WO 2007/005874 and U.S. Pat. No. 7,943,743), or an antibody that comprises the CDRs or variable regions of 3G10, 12A4, 10A5, 5F8, 10H10, 1B12, 7H1, 11E6, 12B7 and 13G4, which are described in PCT Publication WO 07/005874 and U.S. Pat. No. 7,943,743. In certain embodiment an anti-PD-L1 antibody is MEDI4736 (also known as durvalumab and Anti-B7-H1), MPDL3280A (also known as atezolizumab and RG7446), MSB0010718C (also known as avelumab; WO2013/79174), or rHigM12B7. Any of the anti-PD-L1 antibodies disclosed in WO2013/173223, WO2011/066389, WO2012/145493, U.S. Pat. Nos. 7,635,757 and 8.217.149 and U.S. Publication No. 2009/145493 can also be used. Anti-PD-L1 antibodies that compete with and/or bind to the same epitope as that of any of these antibodies can also be used in combination treatments.

In certain embodiments, the CD112R antibody of the disclosure can be used with a CTLA-4 antagonist, e.g., an anti-CTLA-4 antibody. In one embodiment, an anti-CTLA-4 antibody is an antibody selected from the group of: Yervoy® (ipilimumab or antibody 10D1, described in PCT Publication WO 01/14424), tremelimumab (formerly ticilimumab, CP-675,206), monoclonal or an anti-CTLA-4 antibody described in any of the following publications: WO 98/42752; WO 00/37504; U.S. Pat. No. 6,207,156; Hurwitz et al. (1998) Pro. Natl. Acad. Sci. USA 95 (17); 10067-10071; Camacho et al. (2004) J. Clin. Oncology 22 (145); antibodiestract No. 2505 (antibody CP-675206); and Mokyr et al. (1998) Cancer Res. 58:5301-5304. Any of the anti-CTLA-4 antibodies disclosed in WO2013/173223 can also be used.

In some embodiments, a CD112R antibody of the disclosure is used in combination with a LAG-3 (also referred to herein and by others as LAG3) antagonist. Examples of anti-LAG3 antibodies include antibodies comprising the CDRs or variable regions of antibodies 25F7, 26H10, 25E3, 8B7, 11F2 or 17E5, which are described in U.S. Patent Publication No. US2011/0150892, WO10/19570 and WO2014/008218. In one embodiment, an anti-LAG-3 antibody is BMS-986016. Other art recognized anti-LAG-3 antibodies that can be used include IMP731 and IMP-321, described in US 2011/007023, WO08/132601, and WO09/44273. Anti-LAG-3 antibodies that compete with and/or bind to the same epitope as that of any of these antibodies can also be used in combination treatments.

In some embodiments, targeting two or more of TIGIT, CD96 and CD112R receptors simultaneously increases CD226 mediated signaling beyond the anti-CD112R monotherapy. Therefore, in some embodiments, the second agent is an antagonist of TIGIT and/or CD96. Suitable antagonists for use in the combination therapy described herein, include, without limitation, ligands, antibodies (e.g., monoclonal antibodies and bispecific antibodies), and multivalent agents.

In some embodiments, members of the PVR gene family are upregulated on tumor cells and can exhibit intrinsic tumor-promoting properties. Targeting additional members of the PVR gene family in combination with anti-CD112R antibodies leads to enhanced sensitivity to tumors beyond monotherapy. Therefore, in some embodiments, the second agent is selected from one or more of an antagonist of PVRL1, PVRL2, PVRL3, PVRL4, and CD155. Suitable antagonists for use in the combination therapy described herein, include, without limitation, ligands, antibodies (e.g., monoclonal antibodies and bispecific antibodies), and multivalent agents.

STING agonists induce innate immune cell activation resulting in increased T cell priming and recruitment of immune cells into the tumor microenvironment. Targeting STING agonists in combination with CD112R has the potential to lead to an even further increase in T cell and NK cell recruitment and activation.

Increased anti-CD47 antibody mediated phagocytosis can lead to an increase in the presentation of cancer derived antigens by macrophages to T cells. Combination treatment with an anti-CD47 antibody and an anti-CD112R antibody, such as an anti-CD112R antibody provided herein provides an opportunity to enhance cancer antigen specific T cell responses and is fully encompassed herein.

Adenosine, via adenosine receptors expressed on immune cells, inhibits T cell and NK cell activation. Anti-CD39 antibodies inhibit the generation of adenosine by preventing hydrolysis of adenosine triphosphate (ATP). Combination treatment with an anti-CD39 antibody and an anti-CD112R antibody, such as an anti-CD112R antibody provided herein, provides an opportunity to further enhance CD112R therapy by inhibiting adenosine mediated cell signaling in immune cells.

Cytokines can effectively modulate T cell and NK cell activation. IL-27 is an immunosuppressive cytokine that inhibits T cell and NK cell mediated responses. Anti-IL-27 antibodies provide an opportunity to enhance CD112R therapy by limiting immunosuppressive cytokine signaling in immune cells. Thus, combination treatment with an anti-IL-27 antibody and an anti-CD112R antibody, such as an anti-CD112R antibody provided herein, is provided.

The antibodies herein may also be provided before, substantially contemporaneous with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof. For example, a CD112R antibody as described herein could be administered as adjunctive therapy when there is a risk that micrometastases can be present and/or in order to reduce the risk of a relapse.

For treatment of cancer, the combinations may be administered in conjunction with one or more additional anti-cancer agents, such as a chemotherapeutic agent, growth inhibitory agent, anti-cancer vaccine such as a gene therapy vaccine, anti-angiogenesis agent and/or anti-neoplastic composition.

In some embodiments, an anti-inflammatory drug may be administered with the combination, such as a steroid or a non-steroidal anti-inflammatory drug (NSAID). In cases where it is desirable to render aberrantly proliferative cells quiescent in conjunction with or prior to treatment with CD112R antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxy progesteroneacetate, Leuprolide, Flutamide, Toremifene, ZOLADEX®, can also be administered to the subject. When employing the methods or compositions described herein, other agents used in the modulation of tumor growth or metastasis in a clinical setting, such as antimimetics, can also be administered as desired.

Such combination therapies noted above encompass combined administration (where two or more therapeutic agents are included in the same or separate formulations or compositions), and separate administration, in which case, administration of the antibody of the invention can occur prior to, simultaneously, and/or following, administration of the additional therapeutic agent or agents. In some embodiments, administration of the anti-CD112R antibody and administration of an additional therapeutic agent occur within about one month, or within about one, two or three weeks, or within about one, two, three, four, five, or six days, of each other.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention can be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. As used herein, a “split dose” is the division of single unit dose or total daily dose into two or more doses, e.g., two or more administrations of the single unit dose. The antibody may be administered as “split dose.”

The antibody need not be but is optionally formulated with one or more agents currently used to prevent or treat the disorder in question. The effective amount of such other agents depends on the amount of antibody present in the formulation or composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate. In some embodiments, the antibody is provided in a formulation for immediate release and the other agent is formulated for extended release or vice versa.

### G. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-CD112R antibody.

### III. Examples

### Example 1. Anti-CD112R Antibody Generation

Antigens were biotinylated using the EZ-Link Sulfo-NHS-Biotinylation Kit from Pierce. Goat F(ab)2 anti-human kappa-FITC (LC-FITC), ExtrAvidin-PE (EA-PE) and Streptavidin-AF633 (SA-633) were obtained from Southern Biotech, Sigma, and Molecular Probes, respectively. Streptavidin MicroBeads and MACS LC separation columns were purchased from Miltenyi Biotec. Goat anti-human IgG-PE (Human-PE) was obtained from Southern Biotech.

### Primary Discovery.

Eight naïve human synthetic yeast libraries each of ˜109 diversity were propagated as previously described (see, e.g., Y. Xu et al, Addressing polyspecificity of antibodies selected from an in vitro yeast presentation system: a FACS-based, high-throughput selection and analytical tool. PEDS 26.10, 663-70 (2013); WO2009036379; WO2010105256; and WO2012009568.) For the first two rounds of selection, a magnetic bead sorting technique utilizing the Miltenyi MACS system was performed, as previously described (see, e.g., Siegel et al. High efficiency recovery and epitope-specific sorting of an scFv yeast display library.” J Immunol Methods 286 (1-2), 141-153 (2004).) Briefly, yeast cells (˜1010 cells/library) were incubated with 1.5 ml of 10 nM biotinylated Fc-fusion antigen for 15 min at 30° C. in wash buffer (phosphate-buffered saline (PBS)/0.1% bovine serum albumin (BSA)). After washing once with 40 ml ice-cold wash buffer, the cell pellet was resuspended in 20 mL wash buffer, and Streptavidin MicroBeads (500 μl) were added to the yeast and incubated for 15 min at 4° C. Next, the yeast were pelleted, resuspended in 20 mL wash buffer, and loaded onto a Miltenyi LS column. After the 20 mL were loaded, the column was washed 3 times with 3 mL wash buffer. The column was then removed from the magnetic field, and the yeast were eluted with 5 mL of growth media and then grown overnight. The following rounds of selection were performed using flow cytometry. Approximately 2×10⁷ yeast were pelleted, washed three times with wash buffer, and incubated at 30° C., with either decreasing concentrations of biotinylated antigen (100 to 1 nM) under equilibrium conditions, or with a poly-specificity depletion reagent (PSR) to remove non-specific antibodies from the selection. For the PSR depletion, the libraries were incubated with a 1:10 dilution of biotinylated PSR reagent as previously described (see, e.g., Y. Xu et al, Addressing polyspecificity of antibodies selected from an in vitro yeast presentation system: a FACS-based, high-throughput selection and analytical tool. PEDS 26.10, 663-70 (2013).) Yeast were then washed twice with wash buffer and stained with LC-FITC (diluted 1:100) and either SA-633 (diluted 1:500) or EAPE (diluted 1:50) secondary reagents for 15 min at 4° C. After washing twice with wash buffer, the cell pellets were resuspended in 0.3 mL wash buffer and transferred to strainer-capped sort tubes. Selections employing affinity pressure in order to select for and isolate higher affinity antibodies were performed by competing with cold (i.e., unlabeled) antigen.

Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast were plated and individual colonies were picked for characterization.

### Light Chain Batch Shuffle.

Light chain diversification protocol was used during the primary discovery phase for further discovery and improvement of antibodies.

Light chain batch diversification protocol: Heavy chains from a naïve selection output were extracted from the yeast via PCR and transformed into a light chain library with a diversity of 5×10⁶. Selections were performed with one round of MACS and three rounds of FACS as described in the naïve discovery. In the different FACS rounds the libraries were looked at for PSR binding, and affinity pressure by antigen titration down to 0.5 nM. Sorting was performed in order to obtain a population with the desired characteristics.

### Antibody Optimization

Optimization of antibodies was performed by introducing diversities into the heavy chain variable regions as described below.

CDRH1 and CDRH2 selection: The CDRH3 of a single antibody was recombined into a premade library with CDRH1 and CDRH2 variants of a diversity of 1×10⁸ and selections were performed with one round of MACS and three rounds of FACS as described in the naïve discovery. In the different FACS rounds the libraries were looked at for PSR binding, mouse cross-reactivity, and affinity pressure by titration or affinity pressure by pre-complexing the antigen with parental Fab or parental IgG to enrich for binders with higher affinity than the parental IgG. Sorting was performed in order to obtain a population with the desired characteristics.

### Antibody Production and Purification

Yeast clones were grown to saturation and then induced for 48 hours at 30° C., with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect (GE Healthcare LifeSciences).

ForteBio KD measurements

ForteBio affinity measurements were performed on an Octet RED384 generally as previously described (see, e.g., Estep et al, High throughput solution-based measurement of antibody-antigen affinity and epitope binning. Mabs 5 (2), 270-278 (2013)). Briefly, ForteBio affinity measurements were performed by loading IgGs on-line onto AHQ sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model.

### ForteBio Epitope Binning Ligand Blocking

Epitope binning/ligand blocking was performed using a standard sandwich format cross-blocking assay. Control anti-target IgG or receptor was loaded onto AHQ sensors and unoccupied Fc-binding sites on the sensor were blocked with an irrelevant (non-target) human IgG1 antibody. The sensors were then exposed to 100 nM target antigen followed by a second anti-target antibody. Additional binding by the second antibody after antigen association indicates an unoccupied epitope (non-competitor), while no binding indicates epitope blocking (competitor or ligand blocking).

### Biacore Kinetic Assay

For the Biacore-based measurements, the antigen was covalently coupled to a anti mouse-Fc capture C1 chip using an amine-coupling kit (GE Healthcare Bio-Sciences). Association between the antigen and a five-point three-fold titration of the antibody starting at 27 nM was measured for 300 sec. Subsequently, dissociation between the antigen and antibody was measured for 3600 sec. Kinetic data was analyzed and fitted globally using a 1:1 binding model.

### Example 2. Anti-CD112R Antibodies Bind to CD112R

### On Cell Binding Assay

The ability of anti-CD112R antibodies to bind to CD112R expressed on cells was evaluated. 1×10⁵ Jurkat cells (acute T cell leukemia cell line, ATCC #TIB-152) that were either wild type or engineered to over-express human CD112R (Jurkat-CD112R OE) were added to each well of a 96-well V bottom plate and stained with either anti-CD112R antibodies or an IgG1 isotype control (0.63 μg/mL) for 1 hour at 4° C. Cells were washed twice with PBS+2% FCS and resuspended with Alexa Fluor® 647 anti-human IgG Fc antibody (Biolegend, Cat #409320) diluted 1:100 in PBS+2% FCS and incubated at 4° C. for 30 minutes. Cells were subsequently washed twice and resuspended in PBS+2% FCS. Cellular data was acquired using a LSRFortessa X-20 (BD Biosciences) and analyzed with FlowJo software (Tree Star).

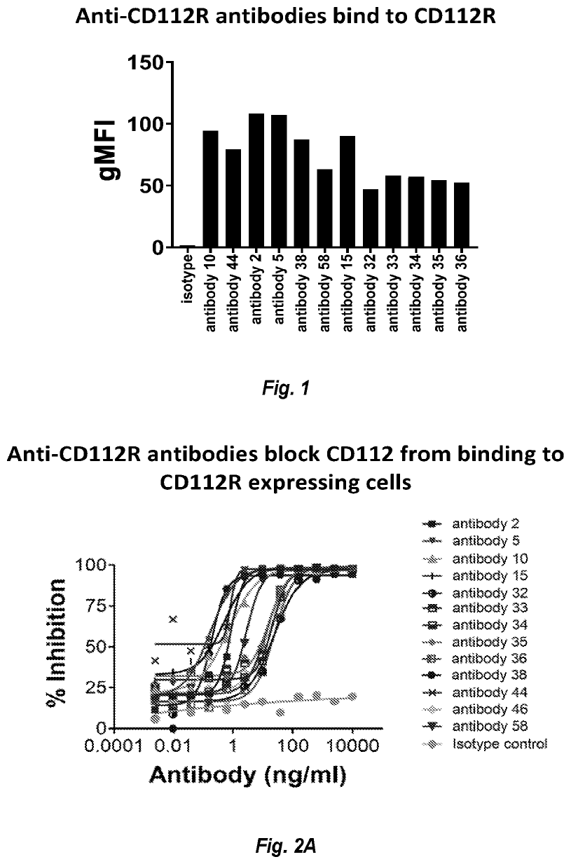

Results are depicted in FIG. 1. Quantitation of antibody binding to Jurkat-CD112R OE cells was assessed by the geometric mean fluorescent intensity (gMFI) of the Alexa Fluor® 647 signal. These results demonstrate that anti-CD112R antibodies bind to cells that express CD112R. A summary of antibody binding is shown in Table 2.

### Example 3. Anti-CD112R Antibodies Block CD112 from Binding to CD112R Expressing Cells

### On Cell Blocking Assay

The ability of anti-CD112R antibodies to block CD112 binding to CD112R expressing cells was evaluated. 1×10⁵ Jurkat cells (acute T cell leukemia cell line, ATCC #TIB-152) that were engineered to over-express human CD112R (Jurkat-CD112R OE) were added to each well of a 96-well V bottom plate and stained with serial dilutions of either anti-CD112R antibodies or an IgG1 isotype control (highest concentration, 10 μg/mL) for 1 hour at 4° C. Cells were washed twice with PBS+2% FCS and resuspended with biotinylated his-tagged human CD112 (3 μg/mL) (BPS Bioscience, #71234) and PE Streptavidin (5 μg/mL) (Biolegend, #405204) in PBS+2% FCS and incubated at 2 hours at 4° C. Cells were subsequently washed twice and resuspended in PBS+2% FCS. Cellular data was acquired using a LSRFortessa X-20 (BD Biosciences) and analyzed with FlowJo software (Tree Star).

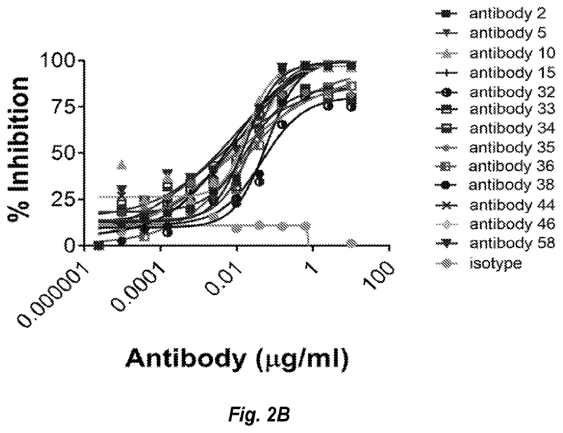

Results are depicted in FIG. 2A and Table 2. Quantitation of CD112 binding to cells was assessed by the geometric mean fluorescent intensity (gMFI) of the PE signal and displayed as percent inhibition. Percent inhibition was calculated as [100−((test sample MFI/Max MFI)*100%)] These results demonstrate that anti-CD112R antibodies inhibit the ability of CD112 to bind to CD112R expressing cells in a dose dependent fashion. A summary of antibody binding and blocking is shown in Table 2.

The ability of anti-CD112R antibodies to block human CD112R was also evaluated by ELISA. Briefly, 96 well Nunc Maxisorp plates were coated with 1 μg/mL of a CD112R-hIgG4 fusion protein in PBS overnight at 4° C. Plates were then washed 6× with PBS+0.01% Tween-20 (PBST) and subsequently blocked with 200 μl of PBS+1% BSA for 1.5 hours at room temperature. After blocking, plates were washed 6× with PBST. Next, 100 μl of anti-CD112R antibodies in PBS+1% BSA was added at a final starting concentration of 10 μg/mL, with 4-fold serial dilutions. Plates were incubated at room temperature for 1.5 hours. Next plates were washed 6× with PBST and then incubated with 1 μg/ml CD112 Fc protein (R&D Systems, Cat #9317-N2-050) that was biotinylated with a sulfo-NHS biotinylation kit (Thermo Fisher, Cat #21925) in 100 μl of PBS+1% BSA for 1 hour at room temperature. Plates were then washed 6× with PBST and subsequently incubated with streptavidin-HRP (Biolegend, Cat #405210) diluted in PBS+1% BSA according to the manufacturer recommendation for 1 hour at room temperature. Following this incubation, plates were then washed 6× with PBST and developed with TMB substrate (Life Technologies, Cat #002023). The reaction was stopped with an equal volume of stop solution (Life Technologies, Cat #SS04). Absorbance at 450 nm (O.D. 450) was measured on a SpectraMax plate reader.

Results are depicted in FIG. 2B and Table 2. These results demonstrate that anti-CD112R antibodies block human CD112R from binding to human CD112. Percent inhibition was calculated as [100−((test sample O.D. 450/Max 0.D. 450)*100%)] Max O.D. 450 was defined as absorbance at 450 nm in the absence of antibody.

TABLE 2
Summary of Antibody Binding and Blocking

| | | Jurkat- | | | |
|---|---|---|---|---|---|
| | | CD112R | | | |
| | | over- | | | |
| | | expressed | | CD112 | |
| | Jurkat | (OE) | CD112 | blocking | CD112 |
| | gMFI | gMFI | blocking | max | blocking |
| | (fold | (fold | IC50 on | inhibition | max |
| | increase | increase | Jurkat- | on Jurkat- | inhibition |
| | over | over | CD112R | CD112R | ELISA |
| Antibody | isotype) | isotype) | OE (ng/mL) | OE (%) | (%) |
| Antibody 2 | 4.7 | 63.9 | 0.83 | 94.1 | 97.0 |
| Antibody 5 | 3.8 | 63.3 | 0.17 | 97.0 | 97.4 |
| Antibody 10 | 4.6 | 55.7 | 0.69 | 96.8 | 96.3 |
| Antibody 15 | 4 | 53.2 | 0.51 | 97.9 | 97.0 |
| Antibody 32 | 2.5 | 27.9 | 23.57 | 96.0 | 75.1 |
| Antibody 33 | 3.4 | 34.2 | 21.89 | 97.4 | 78.8 |
| Antibody 34 | 3.8 | 33.8 | 13.22 | 96.9 | 80.0 |
| Antibody 35 | 3.3 | 32.1 | 16.19 | 97.4 | 81.7 |
| Antibody 36 | 3.5 | 31 | 24.37 | 97.5 | 86.2 |
| Antibody 38 | 3.8 | 51.7 | 0.19 | 95.5 | 97.2 |
| Antibody 44 | 4.8 | 46.9 | 0.81 | 97.7 | 96.8 |
| Antibody 46 | 4.2 | 23.8 | 16.22 | 95.5 | 97.6 |
| Antibody 58 | 4.5 | 37.3 | 2.8 | 96.3 | 97.3 |

### Example 4. Anti-CD112R Antibodies Enhance NK Cell Mediated Killing

### NK Cytotoxicity Assay

To determine the effect of anti-human CD112R antibodies on NK cell mediated cytotoxicity, human NK cells were cocultured with REH target cells (non-T/B cell acute lymphocytic leukemia cell line, ATCC #CRL-8286) in the presence of anti-CD112R antibody or isotype control.

Briefly, NK cells were isolated from the PBMCs of three healthy donors via negative selection (Easysep™ NK cell isolation kit, Stemcell #17955) and activated for 16 hours with IL-2 (10 units/mL) (Peprotech #200-02) and IL-12 (20 ng/ml (Peprotech #200-12) in RPMI+10% FBS+1% Penicillin-Streptomycyin (R10) (ThermoFisher). REH cells were washed, resuspended in PBS (ThermoFisher) and labeled with CellTrace™ violet (CTV) (ThermoFisher #C34557) for 12 minutes at 37° C. Subsequently REH cells were washed with PBS+10% FBS and then resuspended in R10. Following activation, NK cells were washed and resuspended in R10. 2.5×10⁵ NK cells and 5×10⁴ REH cells were added to each well of a 96-well flat bottom plate for an effector-target cell ratio of 5:1. Anti-CD112R and an IgG1 isotype antibody were diluted in R10 and also added to each well at a final concentration of 10 μg/mL. Each condition was run in duplicate. The plates were then incubated for 4 hours at 37° C. Cells were then washed and incubated at room temperature for 30 minutes in the dark with 7-AAD viability dye (1 μg/mL) (Biolegend #420404) to specifically label dead cells. Cell viability data was acquired using a LSRFortessa X-20 (BD Biosciences) and analyzed with FlowJo software (Tree Star). Dead REH cells were defined as CTV and 7-AAD double positive cells.

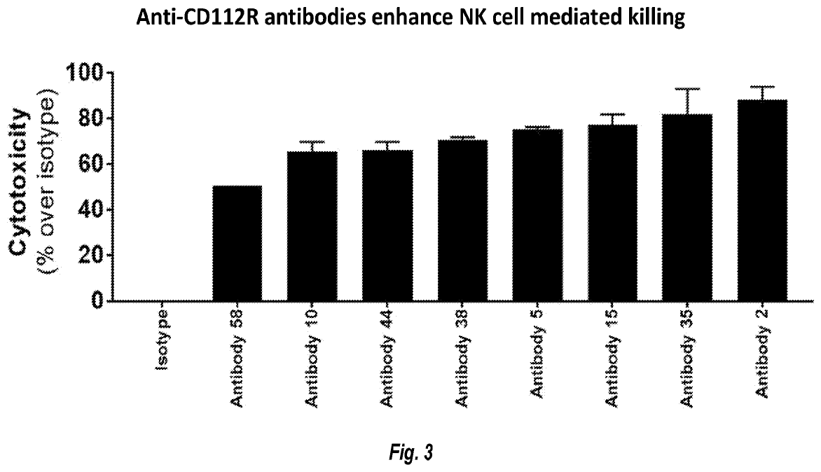

Results are presented in FIG. 3. Cytotoxicity (percent-over isotype) was calculated as ((test percent dead minus isotype percent dead) divided by isotype percent dead)×100. Treatment of NK cells with each of the anti-CD112R antibodies described herein resulted in increased cell mediated cytotoxicity against REH cells compared with an isotype control. These results demonstrate that anti-CD112R antibodies enhance NK cell mediated killing.

### Example 5. Anti-CD112R Antibodies Enhance Antigen Driven Activation of CD8+ T Cells

### Antigen Specific CD8+ T Cell Assay.

The effect of Anti-CD112R antibodies on antigen driven activation of CD8+ T cells was assessed. A primary HLA-A*0201 restricted cytomegalovirus (CMV) specific CD8+ T cell line (Astarte Biologics #1049, Lot #3782DE17) was incubated with peptide pulsed Colo205 cells (Colon Adenocarcinoma cell line, ATCC #CCL-222) in the presence of anti-CD112R antibody or isotype control.

Briefly, CMV specific T cells were thawed, washed and resuspended in X-VIVO 10 (ThermoFisher #BW04380Q). 2×10⁴ CMV T cells were added to each well of a 96-well round bottom plate and rested for 4 hours at 37° C. After the initial rest period, 5×10⁴ Colo205 cells and CMV pp65 peptide (1 ng/ml) (Anaspec #AS-63937) were added to each well. Next, anti-CD112R and isotype antibodies were diluted in X-VIVO 10 and added to each well at a final concentration of 10 μg/mL. Each condition was run in duplicate. Plates were then incubated for 16 hours at 37° C. Supernatants from each well were harvested and subjected to one freeze/thaw cycle prior to cytokine evaluation. After thawing, assay supernatants were diluted 1:5 in X-VIVO 10 and interferon gamma (IFNg) was then measured in the assay supernatant by Luminex Human CD8+ T cell magnetic bead panel multiplex assay (Millipore Sigma #HCD8MAG-15K) and run on a Millipore FlexMap 3D.

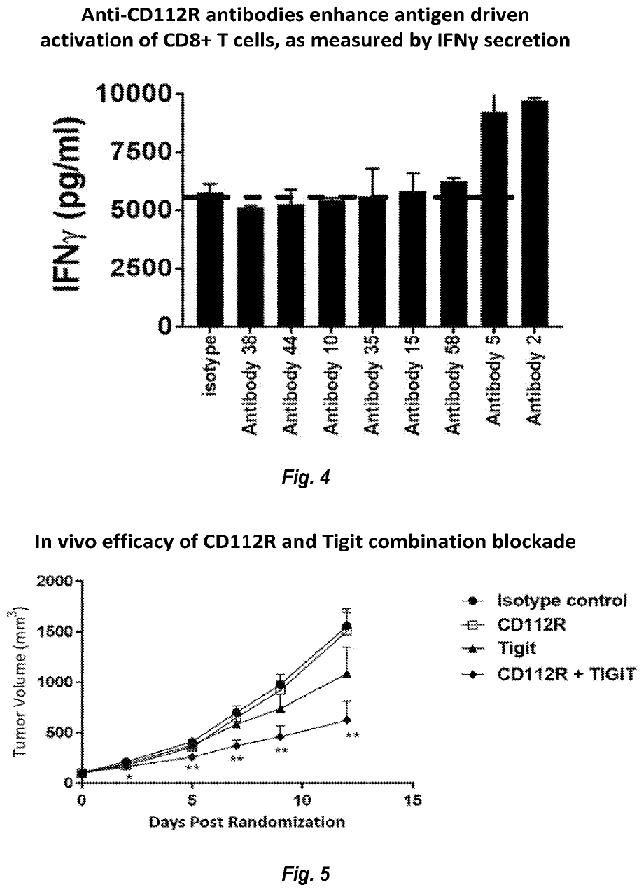

Results are presented in FIG. 4. Test condition IFNg levels were quantitated based on a standard curve generated with defined IFNg concentrations. CD8+ T cells treated with anti-CD112R antibodies 2 and 5 resulted in greater IFNg secretion than observed with isotype control. These results demonstrate that anti-CD112R antibodies enhance antigen driven CD8+ T cell activation.

### Example 6. Combination of Anti-Mouse CD112R and Anti-Mouse TIGIT Antibodies has Therapeutic Effect in the Mouse CT-26 Tumor Model

### In Vivo Efficacy of CD112R and Tigit Combination Blockade

Efficacy of CD112R and TIGIT blockade as single and combination agents was tested in a CT26 colon adenocarcinoma syngeneic mouse tumor model. Balb/c female mice of 7 weeks of age (Charles River Laboratories, #028) were implanted subcutaneously with 0.1×10⁶ CT26. WT cells (ATCC #CRL-2638) in 0.1 mL 50% matrigel inoculation matrix. Mice were randomized into groups of 10 mice each (total of 40 mice) in a stratified manner at tumor volume range of 80-120 mm³ and treated twice weekly for two weeks by intraperitoneal injection as in Table 3.

TABLE 3
Treatment group details

| | Treatment 1 | | | |
|---|---|---|---|---|
| | | Dose | Treatment 2 | |
| | | (μg/ | | Dose |
| Group | Antibody | mouse) | Antibody | (μg/mouse) |
| Isotype | Isotype 1 | 500 | Isotype 2 | 500 |
| CD112R | Isotype 1 | 500 | Anti-CD112R | 500 |
| TIGIT | Anti-TIGIT | 500 | Isotype 2 | 500 |
| CD112R + | Anti-CD112R | 500 | Anti-TIGIT | 500 |
| TIGIT | | | | |

Tumor volumes were measured every 2-3 days until tumors reached IACUC limit size (<2000 mm³).

TABLE 4
Antibody details

| Group | Clone |
|---|---|
| Isotype 1 | Clone C1.18.4, Mouse |
| | IgG2a Isotype Control |
| Isotype 2 | Polyclonal Human IgG |
| | Isotype control |
| Anti-Tigit | 10A7, mouse IgG2a |
| Anti-CD112R | Anti-CD112R human |
| | IgG4 |

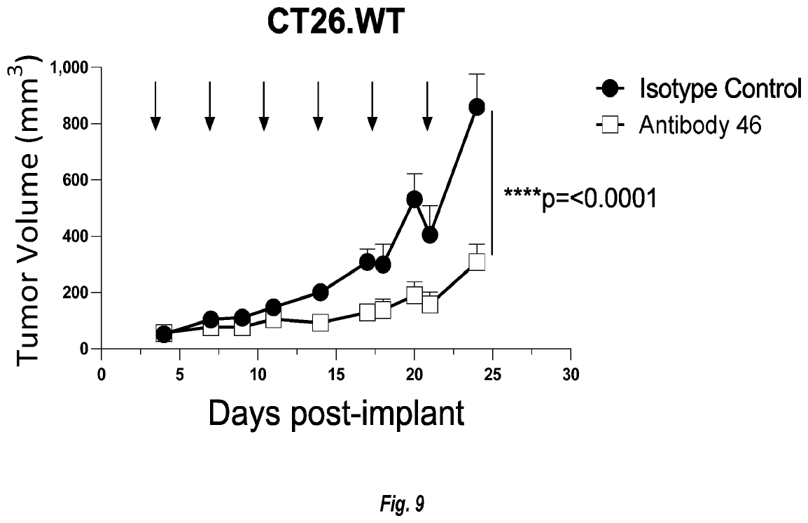

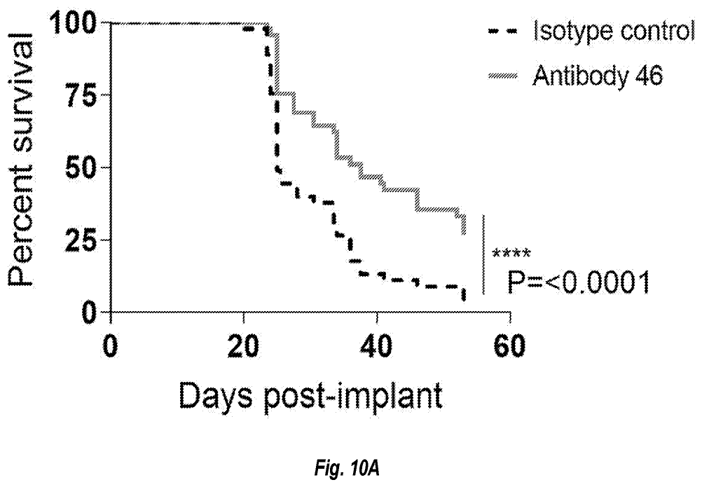

Results are presented in FIG. 5. Graph depicts mean tumor volumes for each treatment group as a function of time. The results demonstrate that the combination of anti-CD112R with anti-TIGIT was effective at reducing tumor growth compared to isotype treated animals while anti-CD112R or anti-TIGIT monotherapies showed either no activity or only a modest effect on reducing tumor growth. While anti-CD112R alone did not show activity in this assay, other experiments presented herein show benefit of anti-CD112R monotherapy. See, for example, FIG. 9.

### Example 7. Increased Expression of CD112R in PBMC Following Anti-CD3 Activation

### CD112R is Upregulated in Activated PBMCs In Vitro

To determine the effect of cellular activation on CD112R expression, human PBMCs were stimulated in vitro with anti-CD3 antibody. Peripheral blood mononuclear cells (PBMCs) from healthy donors were isolated from buffy coats (Research Blood Components). Individual buffy coats were processed separately. 15 mL of buffy coat was added to each 50 ml conical tubes (Corning #430290) and diluted with 15 mL PBS (Thermofisher #14190144)+2 mM EDTA (Fisher Scientific #BP2482-500) for a total volume of 30 mL each tube. Diluted buffy coats were underlain with 14 mL of Ficolpaque (GE Healthcare Life Science #17-544203) and centrifuged at 2000 RPM for 20 minutes at room temperature with the brake turned off. Gradient interphase was collected and washed twice with PBS+2 mM EDTA. Isolated cells were counted and resupended at 2.5-5×10⁷ cell/mL in 10% DMSO (Sigma-Aldrich #472301)+90% heat-inactivated FBS (ThermoFisher #16140-071).

Frozen PBMCs were thawed quickly and resuspended in supplemented RPMI media, which contained RPMI+GlutaMax (1×) (ThermoFisher #61870-036), 10% heat-inactivated FBS, 1×MEM Non-essential amino acids solution (ThermoFisher #15140-122), 1 mM Sodium Pyruvate (ThermoFisher #11360070), 100 U/mL Pen/Strep (ThermoFisher #15140-122), 1×2-mercaptoethanol (ThermoFisher #21985023), 10 mM Hepes (ThermoFisher #15630-080). Isolated PBMCs were washed, counted and resuspended at concentration 0.5×10⁶ cells/mL. 1×10⁶ cells per well were placed in a 24-well flat-bottom plate (Corning #3526) and stimulated with 0.25 μg/mL of anti-CD3 antibody (clone UCHT1, Biolegend #300414). Cells were collected at the indicated time-points, washed in FACS buffer containing 1×PBS, 2% FBS and 2 mM EDTA, and transferred onto a 96-well V-bottom plate (Costar #3894) for antibody staining.

Cells were spun down at 1500 RPM for 3 minutes and the supernatant was removed by flicking. Cells were resuspended in FACS Buffer and incubated for 1 hour at 4° C., with primary antibodies in Table 5 as follows:

TABLE 5

| Antibody | Clone | Company |
|---|---|---|
| CD3-A700 | SK7 | Biolegend # 344822 |
| CD8-FITC | RPA-T8 | Biolegend # 301006 |
| CD4-PEcy7 | RPA-T4 | Biolegend # 300511 |
| CD19-PEdazzle | HIB19 | Biolegend # 302251 |
| NKp46-PE | 9E2 | Biolegend # 331907 |
| CD11b-BV785 | ICRF44 | Biolegend # 301345 |
| PD1-BV421 | EH12.1 | Biolegend #565935 |
| CD226-BV711 | DX11 | BD Biosciences # 564796 |
| CD112R | Internal | Internal |
| Human IgG Fc | HP6017 | Biolegend # 409320 |

Cells were washed twice and incubated for 30 minutes at 4° C., with 1:100 diluted Alexa 647-conjugated anti-Human IgG antibody (clone HP6017, Biolegend #409320). Cells were washed once with FACS buffer and stained with 1:500 diluted Live/Dead Aqua viability dye in 1×PBS (Thermofisher #L34966) for 10 minutes at 4° C. Cells were washed once and acquired directly on flow cytometer X-20 Fortessa (BD Biosciences). Data was analyzed using Flowjo (TreeStar) and Graphpad prism (Graphpad Software).

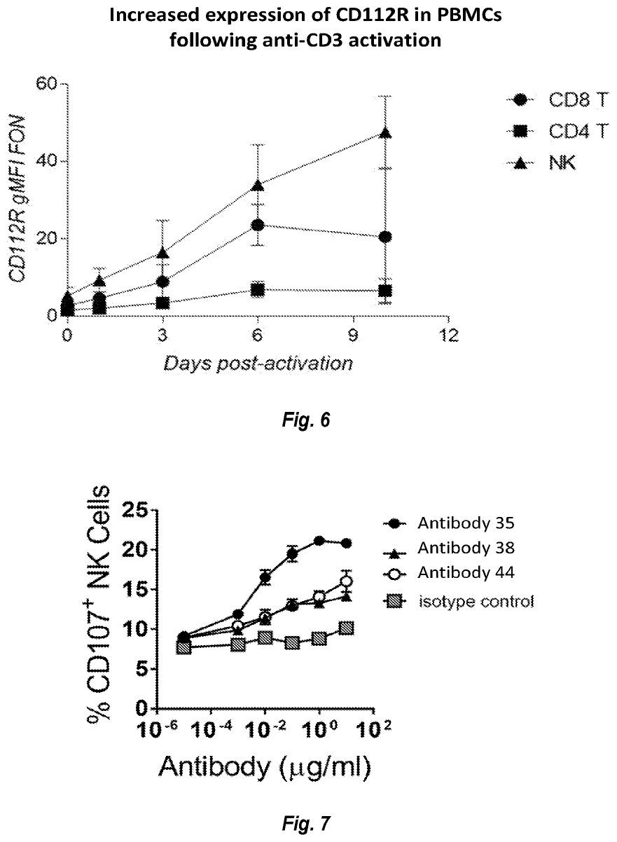

Results are depicted in FIG. 6. Quantitation of CD112R antibody binding was assessed by the geometric mean fluorescent intensity (gMFI) of the Alexa Fluor® 647 signal for the indicated cell type. Anti-CD112R binding is depicted as fold over negative (FON. (CD112R gMFI divided by isotype gMFI)). These results demonstrate that CD112R expression increases on NK cells and T cells following anti-CD3 activation.

### Example 8. Anti-CD112R Antibodies Enhance NK Cell Degranulation in Tumor Cell Co-Cultures

To determine the effect of anti-CD112R antibody on NK cell mediated degranulation, human NK cells were cocultured with Raji target cells (Burkitt lymphoma cell line. ATCC #CCL-86) that had been previously transduced with lentivirus to express CD112 (Origene. #RC213693L2), in the presence of antibodies 35, 38, 44 and isotype control.

Briefly. NK cells were isolated and pooled from the PBMCs of three healthy donors via negative selection (Easysep™ NK cell isolation kit. Stemcell #17955) and cultured for 16 hours in DMEM+10% FBS+1% Penicillin-Streptomycyin (D10) (ThermoFisher). Following overnight incubation at 37° C. NK cells were washed and resuspended in D10. Raji.CD112 cells were harvested, washed and then resuspended in D10. 1×10⁵ NK cells and 5×10⁴ Raji.CD112 cells were added to each well of a 96-well flat bottom plate for an effector-target cell ratio of 2:1. Anti-CD112R antibodies and an IgG1 isotype control antibody were diluted in D10 and added to each well at starting concentration of 10 μg/mL, with 10-fold serial dilutions. Each condition was run in duplicate. PE anti-CD107a antibody (Biolegend. #328608) and Monensin (Biolegend. #420701) were also added to each well at the manufacturer's indicated concentrations. The final volume for each well was 200 μl. The plates were then incubated for 4 hours at 37° C. After 4 hours. Anti-CD3 FITC (Biolegend. #300306) and Anti-NKp46 APC (Biolegend #331914) antibodies were diluted in D10 and 50 μl was added to each well. The plates were then incubated for an additional 30 minutes at 4° C., to stain. Cells were then transferred to V bottom plates and washed twice and resuspended in PBS+2% FBS. Data was acquired using a LSRFortessa X-20) (BD Biosciences) flow cytometer and analyzed with FlowJo software (Tree Star). NK degranulation was defined as the frequency of CD107a cells within the CD3⁻ NKp46⁺ lymphocyte gate.

Results are presented in FIG. 7. Treatment of NK cells with anti-CD112R antibodies described herein resulted in increased NK degranulation, as measured by CD107a staining, compared with an isotype control.

### Example 9. Anti-CD112R Antibodies Increase NK Cell Activation in PBMC Tumor Cell Cocultures

To assess the impact of CD112R antibodies on NK cell activation, several antibodies were evaluated in PBMC-tumor cell cocultures. Upregulation of CD137 (4-1BB), which has been previously established as a marker of NK cell activation (Baessler et al. (2010) Blood 115 (15); André et al. (2018) Cell 175, 1731-1743) was measured on the NK cells from PBMCs cocultured with K562 target cells (chronic myelogenous leukemia cell line. ATCC #CCL-243) with anti-CD112R or isotype control antibodies.

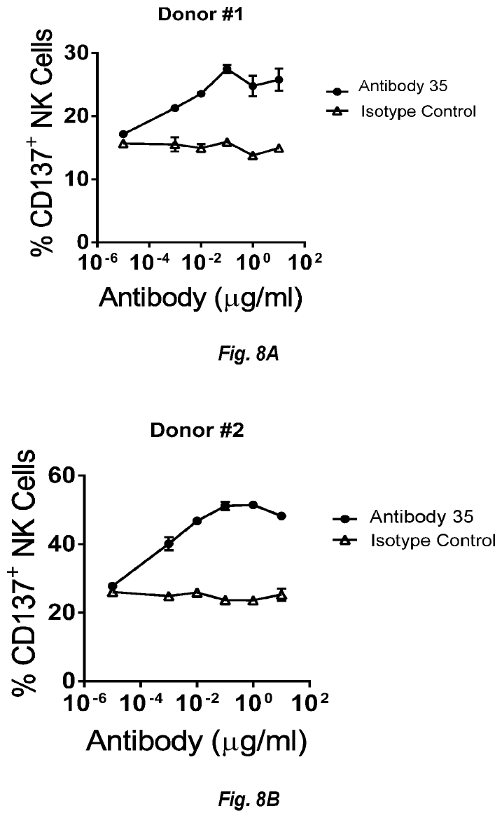

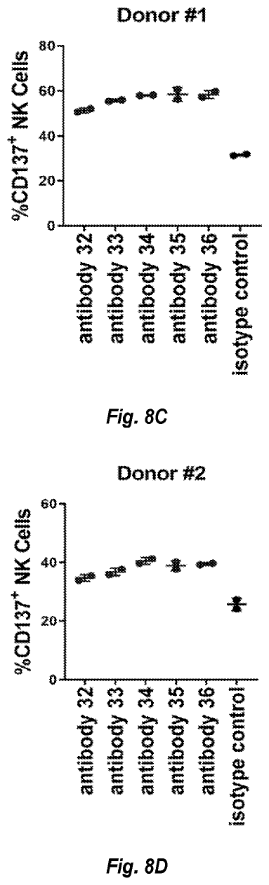

Briefly, frozen PBMCs isolated from the buffy coats of healthy donors were thawed, washed, resuspended in DMEM+10% FBS+1% Penicillin-Streptomycyin (D10) and plated into 96 well flat bottom plates at a concentration of 5×10⁵ cells per well and rested for 4 hours at 37° C. prior to adding target cells and antibodies. Next, in a first experiment (FIG. 8A-8B) CD112R antibodies and an IgG1 isotype control antibody were diluted in D10 and added to each well at starting concentration of 10 μg/mL, with 10-fold serial dilutions. In a next experiment (FIG. 8C-8D) a single concentration (1 μg/mL) of anti-CD112R or IgG1 isotype control antibody was added to each well. For both experiments, each condition was run in duplicate. K562 cells were then harvested, washed and resuspended in D10 and added to each well at a concentration of 5×10⁴ cells per well. The final volume for each well was 200 μl. The plates were then incubated for 16 hours at 37° C. After 16 hours, cells were then transferred to V bottom plates and washed twice in PBS+2% FBS. Cells were stained with Anti-CD3 FITC (Biolegend. #300306). Anti-NKp46 BV421 (Biolegend #331914) and anti-CD137 APC (Biolegend. #309810) in PBS+2% FBS for 30 minutes at 4° C. Cells were subsequently washed twice and resuspended in PBS+2% FBS. Data was acquired using a LSRFortessa X-20 (BD Biosciences) flow cytometer and analyzed with FlowJo software (Tree Star). NK cell activation was defined as the frequency of CD137+ cells within the CD3− NKp46+ lymphocyte gate.

Results from two individual donors from two independent experiments are presented in FIG. 8A-8D. The addition of anti-CD112R antibody to PBMC-K562 cell cocultures resulted in significant activation of NK cells compared to isotype control, as measured by CD137 upregulation on NK cells.

### Example 10. Anti-CD112R Decreases Tumor Growth in CT-26 Model

In vivo efficacy of CD112R blockade was evaluated in the CT26. WT colon adenocarcinoma syngeneic mouse tumor model. BALB/cAnNTac female mice of 7 weeks of age (Taconic Biosciences. Catalog #BALB-F) were implanted subcutaneously in the right flank with 0.2×10⁶ CT26. WT (ATCC. Catalog #CRL-2638) in 0.1 mL 50% Geltrex (GIBCO, catalog #A1432-02) and 50% RPMI-1640) serum-free media (GIBCO, catalog #A10491-01). Mice with palpable tumors were randomized on day 4 post-implantation and treated intraperitoneally twice weekly for three weeks starting on the day of randomization as follows in Table 6.

TABLE 6

| Group | Treatment | Dose (μg/mouse) |
|---|---|---|
| Isotype control | Mouse IgG2a | 500 |
| | isotype control | |
| Antibody 46 | Anti-CD112R | 500 |
| | mouse IgG2a | |

Tumor volumes were measured with a caliper every 2-3 days until tumors reached IACUC limit size (<2000 mm³). Tumor volume (mm³) was calculated as follows: width (mm)×[length (mm)]2×0.5.

Results are presented in FIG. 9. The graph depicts pooled data from three independent experiments showing mean tumor volumes for each treatment group as a function of time. These results demonstrate that the treatment of tumor bearing mice with CD112R antibody resulted in significant inhibition of tumor growth as measured on day 24 post-inoculation.

### Example 11. CD112R Blockade Results in Anti-Tumor Immunity in Mice with Complete Tumor Rejection in the CT26 Model

Anti-tumor immunity was evaluated in anti-CD112R treated mice that exhibited complete responses from primary CT26. WT tumor challenges. For the primary challenge, BALB/cAnNTac female mice of 7 weeks of age (Taconic Biosciences, Catalog #BALB-F) were implanted subcutaneously in the right flank with 0.2×10⁶ CT26.WT (ATCC, Catalog #CRL-2638) in 0.1 mL 50% Geltrex (GIBCO, catalog #A1432-02) and 50% RPMI-1640 serum-free media (GIBCO, catalog #A10491-01). Mice with palpable tumors were randomized on day 4 post-implantation and treated intraperitoneally twice weekly for three weeks starting on the day of randomization as follows in Table 7.

TABLE 7

| Group | Treatment | Dose (μg/mouse) |
|---|---|---|
| Isotype control | Mouse IgG2a | 500 |
| | isotype control | |
| Antibody 46 | Anti-CD112R | 500 |
| | mouse IgG2a | |

Tumor volumes were measured with a caliper every 2-3 days until tumors reached IACUC limit size (<2000 mm³). Tumor volume (mm³) was calculated as follows: width (mm)×[length (mm)]2×0.5.

All Surviving mice at day 50 post implantation that lacked any discernable tumors were considered to be survivors/complete responders. Complete responder mice (n=8) from the anti-CD112R treated group were re-challenged via inoculation in the left flank with 1×10⁶ CT26. WT cells (ATCC. Catalog #CRL-2638) in 0.1 mL 50% Geltrex (GIBCO, catalog #A1432-02) and 50% RPMI-1640) serum-free media (GIBCO, catalog #A10491-01), a five-fold increase from the primary inoculation dose. As a control, age-matched naïve Balb/c female mice (n=5) were also similarly inoculated in the left flank with 1×10⁶ CT26. WT cells in 0.1 mL 50% Geltrex and 50% RPMI-1640) serum-free media. Mice did not receive any further treatment. Tumor volumes were measured every 2-3 days until tumors reached IACUC limit size (<2000 mm3). Tumor volume (mm3) was calculated as follows: width (mm)×[length (mm)]2×0.5.

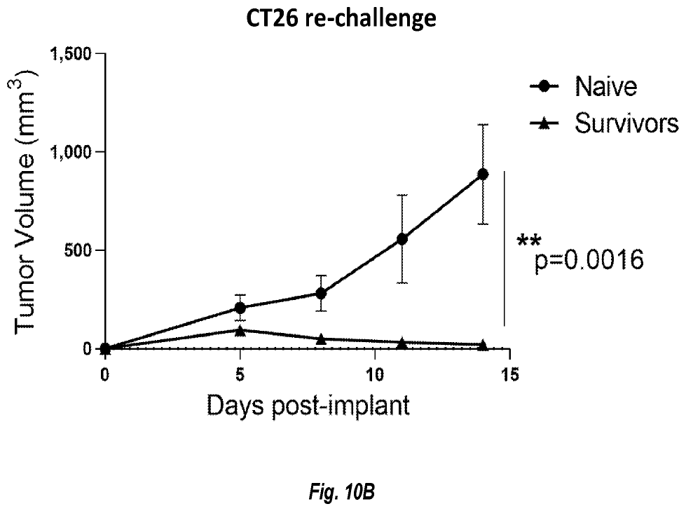

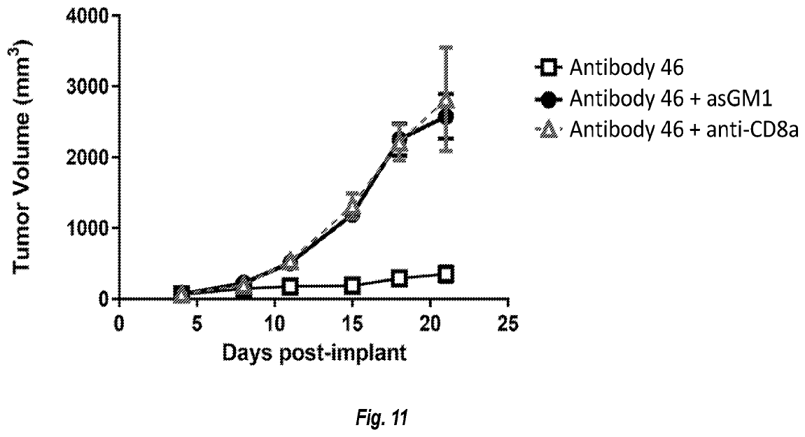

Results are presented in FIG. 10A-10B. FIG. 10A depicts pooled data from 3 independent experiments showing the survival frequency of mice implanted with CT26 primary tumors treated with anti-CD112R. FIG. 11B depicts mean tumor volumes for anti-CD112R treated complete responders following tumor re-challenged and naïve challenged controls as a function of time. Statistical analysis was performed by Mantel-Cox test on day 50) post implant (FIG. 10A) and by Mann-Whitney test on day 15 post implant (FIG. 10B). These results demonstrate that mice treated with anti-CD112R exhibited complete responses following primary tumor challenge and the subsequent rapid rejection upon tumor re-challenge in these mice also demonstrate that treatment with anti-CD112R antibody leads to the development of immunological memory and protective immunity.

### Example 12. Both NK Cells and CD8 T Cells Contribute to Therapeutic Activity of Anti-CD112R in CT26 Tumor Challenge

In vivo efficacy of CD112R blockade was evaluated in the CT26 syngeneic mouse tumor model following NK cell or CD8 T cell depletion. To deplete NK and CD8 T cells, mice were treated twice weekly for three weeks starting at randomization with Asialo-GM1 antibody (“asGM1” in FIG. 11; Biolegend; cat #146002; dose 100 uL/mouse; intraperitoneally) and anti-CD8a antibody (Bioxcell; cat #BE0085; 200 μg/mouse; intraperitoneally) respectively.

BALB/cAnNTac female mice of 7 weeks of age (Taconic Biosciences. Catalog #BALB-F) were implanted subcutaneously in the right flank with 0.2×10⁶ CT26.WT (ATCC, Catalog #CRL-2638) in 0.1 mL 50% Geltrex (GIBCO, catalog #A1432-02) and 50% RPMI-1640 serum-free media (GIBCO, catalog #A10491-01). Mice with palpable tumors were randomized on day 4 post-implantation and treated intraperitoneally twice weekly for three weeks starting on the day of randomization with antibody 46 (anti-CD112R mouse IgG2a; 12.5 mg/kg; intraperitoneally).

Tumor volumes were measured with a caliper every 2-3 days until tumors reached IACUC limit size (<2000 mm³). Tumor volume (mm³) was calculated as follows: width (mm)×[length (mm)]²×0.5.

Results are presented in FIG. 11. The graph depicts mean tumor volumes for each treatment group as a function of time. These results demonstrate that the therapeutic effect of anti-CD112R is significantly diminished following NK cell or CD8 T cell depletion. These results indicate that both CD8 T cells and NK cells are required for effective tumor growth inhibition mediated by anti-CD112R.

### Example 13. CD112R Blockade Activates Tumor NK Cells In Vivo

Ex Vivo Assessment of NK Activation Markers after Dosing with Anti-CD112R Monotherapy.

To determine the effects of anti-CD112R antibody on NK cell activation in vivo, BALB/cAnNTac female mice of 7 weeks of age (Taconic Biosciences, Catalog #BALB-F) were implanted subcutaneously in the right flank with 0 2×10⁶ CT26 WT (ATCC. Catalog #CRL-2638) in 0.1 mL 50% Geltrex (GIBCO, catalog #A1432-02). Mice with palpable tumors were randomized on day 4 post-implantation and administered with 500 μg of either isotype control antibody (clone C1.18.4, BioXcell, cat #0085) or antibody 46 (anti-CD112R mouse IgG2a). Both groups were also co-adminstered with 500 μg of an isotype control (clone MOPC-21, Bioxcell, cat #0083). Treatments were prepared in sterile 1×PBS (GIBCO cat #14190-136) and a total volume of 100 μL was injected intraperitoneally

Tumor processing: Mice were euthanized and tumors were resected 24 hours post-treatment. Tumor were processed into single-cell suspensions by breaking tissue over a 440-micron mesh filter (Costar #3480) placed over a 50 mL centrifuge tube (Falcon #352350) using the rough end of a 3 mL syringe plunger (BD 301077) in FACS buffer (1×PBS, 2% heat-inactivated FBS, GIBCO cat #16140-071; 2 mM EDTA, Fisher Bioreagents, cat #BP2482-500). Dislodged tumors were filtered once more over a 70 micron strainer (Falcon cat #352350) and any remaining tissue was further broken down using a 3 mL syringe plunger. Cell were spun at 800 g for 10 minutes. Cell pellets were resuspended in FACS buffer.

Ex vivo re-stimulation: Roughly half of the single-cell suspension was transferred into a 96-well U-bottom polypropylene 2 mL deep plates (Thermofisher #AB-0932) and centrifuged at 1000 g for 5 minutes at 4° C. Cells were resuspended in pre-warmed 1×RPMI+Glutamax (GIBCO #61870-035) medium with 10% heat-inactivated FBS, containing 20 ng/mL PMA (Abcam #ab120297), 500 ng/ml Ionomycin Ca²⁺ salt (Abcam #ab 120116), 5 μg/mL Brefeldin A (Biolegend #420601) and 2 μM Monensin (Biolegend #420701). Cells were incubated for 3.5 hours at 37° C., 5% CO₂. Cells were centrifuged again as described above.

Surface antigen antibody staining for FACS: Cell pellets were washed once with 500 μL of cold FACS buffer. Cells were resuspended in 100 μL of FACS buffer with TruStain fcX™ (anti-mouse CD16/32, at dilution indicated in Table 8. Cells were incubated on ice for 15 minutes. Surface antibody cocktail was prepared (see Table 8 for details) and added directly to pre-blocked cells. Cells were incubated for 1 hour on ice. Cells were then washed twice with 500 μL of FACS buffer.

Viability dyes staining: Cells stained with Live/Dead Aqua viability dye diluted 1:500 in 1×PBS (Thermofisher #L34966) for 10 minutes at 4° C.

Fixation: Cells were washed once and fixed with 200 μL of eBioscience Foxp3 Fixative/permeabilization buffer (Thermofisher #00-5523-00, using manufacturer's protocol for dilution guidelines) overnight at 4° C.

Intracellular antigen antibody staining for FACS: Cells were permeabilized by adding directly 200 μL of 1× eBioscience permeabilization buffer (see manufacturer's protocol for dilution guidelines). Cells were centrifuged at 1000g for 5 minutes and stained with intracellular panel of antibodies at final dilution in 1× eBioscience permeabilization buffer. Cells were incubated for 1 hour at room temperature. Cells were washed twice with 500 μL of permeabilization buffer, resuspended in 150 μL of FACS buffer.

Cells were acquired on X-20 Fortessa flow cytometer (BD Biosciences). Data was analyzed using Flowjo (Flowjo, LLC) and Graphpad prism (Graphpad Software).

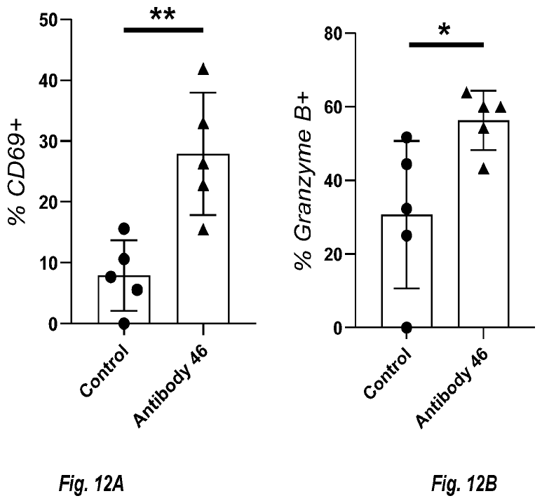

TABLE 8
Antibodies used in FIG. 12 A-B.

| | | | | | | Type of |
|---|---|---|---|---|---|---|
| Fluorophore | Antibody | Clone | Company | Catalog # | Dilution | stain |
| N/A | FcBlock | 93 | Biolegend | 101320 | 1:100 | Fc block |
| Alexa700 | CD45 | I3/2.3 | Biolegend | 147716 | 1:100 | Surface |
| PEDazzle | NKp46 | 29A1.4 | Biolegend | 137630 | 1:100 | Surface |
| APC/Fire ™ | TCRbeta | H57- | Biolegend | 109246 | 1:100 | Surface |
| | | 597 | | | | |
| BV785 | CD69 | H1.2F3 | Biolegend | 101243 | 1:100 | Surface |
| BV510 | Live/Dead | | Thermofisher | L34966 | 1:500 | Viability |
| APC | Gzmb | NGZB | Thermofisher | 17-8898-82 | 1:100 | Intracellular |

Results are depicted in FIG. 12A-B. FIG. 12A shows the frequency of tumor infiltrating NK cells expressing CD69. FIG. 12B shows the frequency of tumor infiltrating NK cells expressing Granzyme B following ex vivo restimulation. NK cells were gated as follows: CD45⁺, CD45⁺ SSC-A^{Low}, Live, Singlets, NKp46⁺ TCRb⁻ population. Positive gate was set based on negative controls fluorescence-minus one (FMO). P-value was derived using unpaired t-test (*, p<0.05; **, p<0.01, ***, p<0.001). These results demonstrate that CD112R blockade significantly increases expression of the early activation marker CD69 and the cytotoxic granule protein, granzyme B, in intratumoral NK cells in CT-26 tumor model.

### Example 14. Combination of Anti-CD112R and Anti-PD1 Antibodies has Therapeutic Effect and Increased Tumor-Free Survival in the Mouse CT-26 Tumor Model

### In Vivo Efficacy of CD112R and PD-1 Combination Blockade

Efficacy of CD112R and PD-1 blockade as a monotherapy and as a combination therapy was tested in CT-26 colon adenocarcinoma syngeneic tumor model. BALB/cAnNTac female mice of 6 weeks of age (Taconic Biosciences, Balb-F) were implanted subcutaneously in the right flank with 0.2×10⁶ CT26. WT (ATCC, Catalog #CRL-2638) in 0.1 mL 50% Geltrex (GIBCO, catalog #A1432-02) and 50% RPMI-1640 serum-free media (GIBCO, catalog #A10491-01). Mice were randomized into groups of 10 mice each with mean tumor volume of 90 mm³ using the matched distribution randomization method. Mice were treated twice weekly for two weeks by intraperitoneal injection as in Table 9. Details about treatment agents are included in Table 10.

TABLE 9
Treatment group details

| | Treatment 1 | | Treatment 2 | |
|---|---|---|---|---|
| | | Dose | | Dose |
| Group | Antibody | (mg/kg) | Antibody | (mg/kg) |
| Isotype | Isotype 1 | 12.5 | Isotype 2 | 10 |
| CD112R | Anti-CD112R | 12.5 | Isotype 2 | 10 |
| PD-1 | Isotype 1 | 12.5 | Anti-PD-1 | 10 |
| CD112R + PD-1 | Anti-CD112R | 12.5 | Anti-PD-1 | 10 |

TABLE 10
Antibody details

| Group | Clone |
|---|---|
| Isotype 1 | Isotype Control Clone C1.18.4, |
| | Mouse IgG2a |
| Isotype 2 | Isotype Control Clone 2A3, Rat |
| | IgG2a |
| Anti-CD112R | Antibody 46, Mouse IgG2a |
| Anti-PD-1 | RMP1-14, Rat IgG2a |

Tumor volumes were measured twice weekly until tumors reached IACUC limit size (<2000 mm3). Tumor volume (mm3) was calculated as follows: width (mm)×[length (mm)]²×0.52.

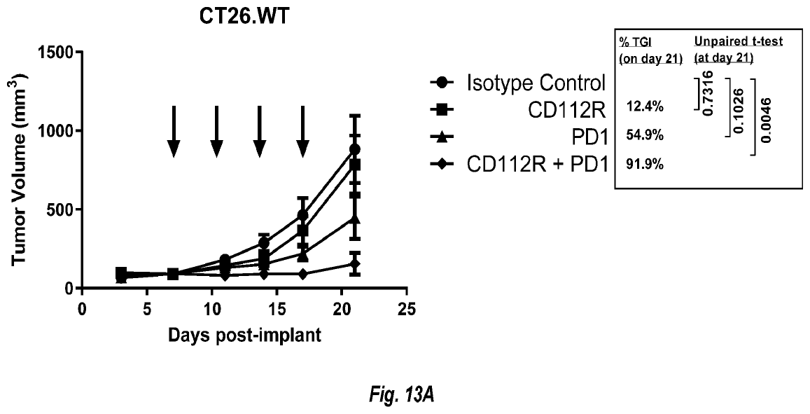

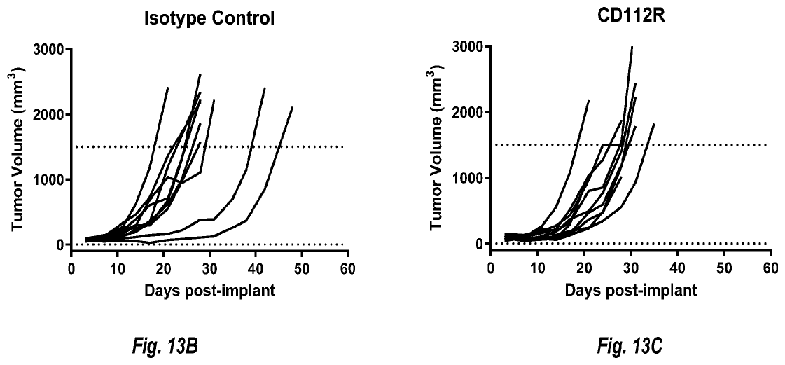

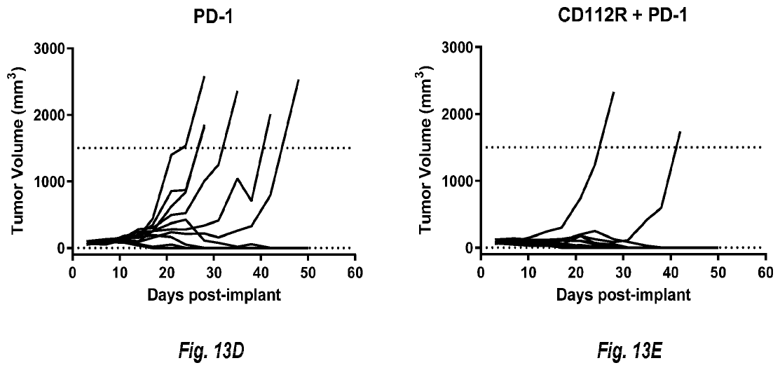

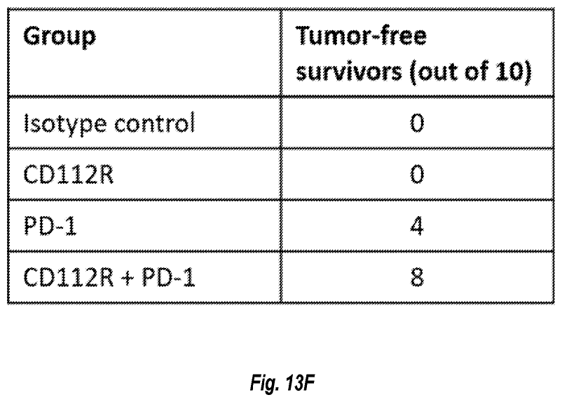

Results are presented in FIG. 13A-F. FIGS. 13A-E depict mean and individual tumor volume measurements respectively for each treatment group as a function of time. The results shown in FIG. 13A demonstrate that the combination of anti-CD112R with anti-PD-1 was effective and statistically significant (as measured on day 21 by unpaired t-test) at reducing tumor growth compared to isotype treated animals. Anti-CD112R or anti-PD-1 monotherapies also showed activity in reducing tumor growth. FIG. 13F depicts overall tumor-free survival on day 50 post-implantation indicated as fraction of tumor-free survivors per group after treatment as described above. These results demonstrate that the combination of anti-CD112R with anti-PD-1 confers higher tumor-free survival rate than isotype control or Either Monotherapeutic Agent.

### Example 15. Binding of anti-CD112R antibodies to cells expressing murine CD112R

The ability of anti-CD112R antibodies to bind to mouse CD112R was evaluated on cells overexpressing mouse CD112R. 0.8×10⁵ 293T cells (ATCC CRL-3216) that were engineered to overexpress mouse CD112R (293T.mCD112R) were added to each well of a 96-well V bottom plate and stained with either anti-CD112R antibodies or an IgG1 isotype control at a starting concentration of 10 μg/mL, with 3-fold serial dilutions for 30 minutes at 4° C. Cells were washed twice with PBS+2% FCS and resuspended with Alexa Fluor® 647 anti-human IgG Fc antibody (Biolegend, Cat #409320) diluted 1:100 in PBS+2% FCS and incubated at 4° C. for 20 minutes. Cells were subsequently washed twice and resuspended in PBS+2% FCS. Cellular data was acquired using a LSRFortessa X-20 (BD Biosciences) and analyzed with FlowJo software (Tree Star).

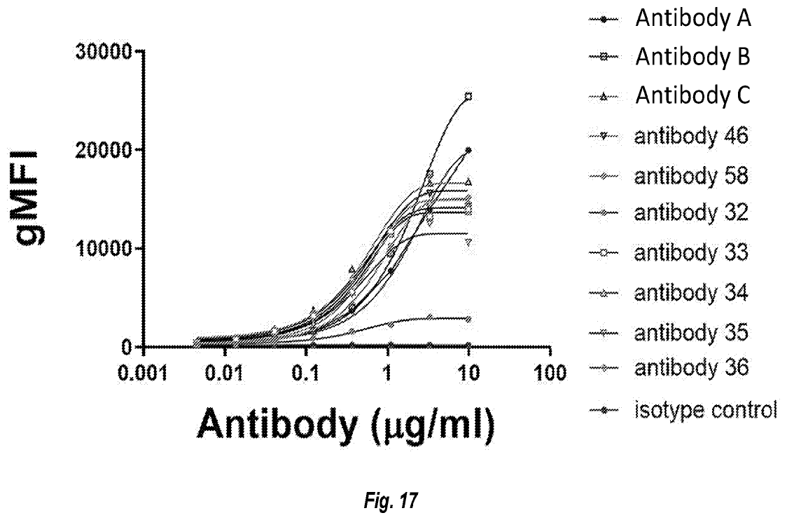

Results are depicted in FIG. 17 and Table 11. Quantitation of antibody binding to 293T.mCD112R cells was assessed by the geometric mean fluorescent intensity (gMFI) of the Alexa Fluor® 647 signal. These results demonstrate that several anti-CD112R antibodies bound to cells that expressed mouse CD112R.

### Example 16. Binding of Anti-CD112R Antibodies to Soluble Murine CD112R

The ability of anti-CD112R antibodies to bind to soluble mouse CD112R was evaluated by ELISA. Briefly. 96 well Nunc Maxisorp plates were coated with 1 μg/mL of anti-CD112R antibodies or isotype control (Biolegend. Cat #403502) in PBS overnight at 4° C. Plates were then washed 6× with PBS+0.01% Tween-20 (PBST) and subsequently blocked with 200 μL of PBS+1% BSA for 1.5 hours at room temperature. After blocking, plates were washed 6× with PBST. Next. 100 μL of a mouse CD112R-hIgG4 fusion protein in PBS+1% BSA was added at a final starting concentration of 10 μg/mL, with 4-fold serial dilutions. Plates were incubated at room temperature for 1.5 hours. Next plates were washed 6× with PBST and then incubated with anti-IgG4 HRP (Thermo Fisher, Cat #MA1-33437) in 100 μL for 1 hour at room temperature. Plates were then washed 6× with PBST and developed with TMB substrate (Life Technologies. Cat #002023). The reaction was stopped with an equal volume of stop solution (Life Technologies. Cat #SS04). Absorbance at 450 nm (O.D. 450) was measured on a SpectraMax plate reader.

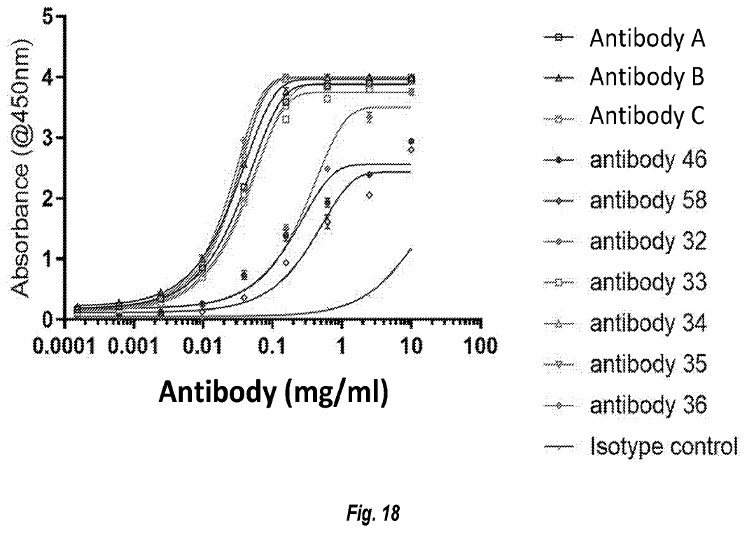

Results are depicted in FIG. 18 and Table 11. These results demonstrate that anti-CD112R antibodies bound to soluble mouse CD112R.

### Example 17. Inhibition or Blocking of Murine CD112R Binding to CD112

The ability of species cross reactive anti-CD112R antibodies to block mouse CD112R binding to mouse CD112 was evaluated by ELISA. Briefly. 96 well Nunc Maxisorp plates were coated with 1 μg/mL of mouse CD112 (Sino Biological. Cat #50318-M08H) in PBS overnight at 4° C. Plates were then washed 6× with PBS+0.01% Tween-20 (PBST) and subsequently blocked with 200 μL of PBS+1% BSA for 1.5 hours at room temperature. After blocking, plates were washed 6× with PBST. Next. 50 μL of anti-CD112R antibodies or isotype control (Biolegend. Cat #403502) in PBS+1% BSA were added at a final starting concentration of 40 μg/mL, with 2-fold serial dilutions. 50 μL of a mouse CD112R-hIgG4 fusion protein was also added to each well at a final concentration of 2 μg/mL. Plates were incubated at room temperature for 1.5 hours. Next plates were washed 6× with PBST and then incubated with anti-IgG4 HRP (Thermo Fisher, Cat #MA1-33437) in 100 μL for 1 hour at room temperature. Plates were then washed 6× with PBST and developed with TMB substrate (Life Technologies. Cat #002023). The reaction was stopped with an equal volume of stop solution (Life Technologies. Cat #SS04). Absorbance at 450 nm (O.D. 450) was measured on a SpectraMax plate reader.

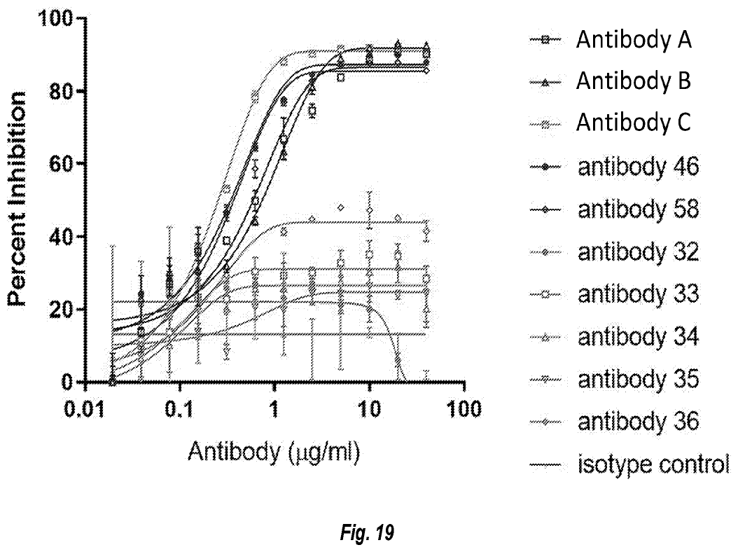

Results are depicted in FIG. 19 and Table 11. These results demonstrate that anti-CD112R antibodies inhibited mouse CD112R binding to mouse CD112 to varying degrees. Percent inhibition was calculated as [100−((test sample O.D. 450/Max O.D. 450)*100%)] Max O.D. 450 was defined as absorbance at 450 nm in the absence of antibody.

As shown in Table 11 and FIGS. 17-19, antibodies 32, 33, 34, 35 and 36 are capable of blocking the interaction of human CD112 to human CD112R, but according to the definition of blocking described herein, not capable of blocking the binding interaction between mouse CD112R and mouse CD112. See, e.g., the last two columns of Table 11 for antibodies 32, 33, 34, 35 and 36, showing % inhibition of the mouse interaction at 0, 28.3, 20.3, 24.2, and 41.6, respectively, as compared to the % inhibition of the human interaction at 75.1, 78.8, 80, 81.7, and 86.2, respectively. Antibodies not belonging to the exemplary class of antibodies related to antibody 32 do not exhibit such differential blocking.

TABLE 11
Summary of Antibody Binding and Blocking Antibody
Details (mouse)

| | | | | human |
|---|---|---|---|---|
| | 293T-mouse | Mouse | mouse | CD112/ |
| | CD112R | CD112R | CD112/ | CD112R |
| | gMFI (fold | Binding | CD112R | blocking |
| | increase | ELISA | blocking | ELISA max |
| | over | EC50 | ELISA max | inhibition |
| Antibody | isotype) | (ng/ml) | inhbition (%) | (%) |
| Antibody A | 104.6 | 34.5 | 90.3 | 90.6 |
| Antibody B | 133.3 | 27.4 | 92.5 | 88.9 |
| Antibody C | 88.1 | 41.0 | 90.1 | 97.8 |
| Antibody 32 | 14.9 | 269.5 | 0.0 | 75.1 |
| Antibody 33 | 73.3 | 38.3 | 28.3 | 78.8 |
| Antibody 34 | 72.2 | 24.2 | 20.3 | 80.0 |
| Antibody 35 | 55.6 | 21.5 | 24.2 | 81.7 |
| Antibody 36 | 76.3 | 25.1 | 41.6 | 86.2 |
| Antibody 46 | 74.8 | 180.6 | 88.0 | 97.6 |
| Antibody 58 | 79.4 | 411.7 | 85.7 | 97.3 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Table of Sequences

| SEQ | | | |
|---|---|---|---|
| ID | Clone | | |
| NO | No | Description | Sequence |
| 1 | 2 | VH CDR1 | FTFSEYTMN |
| 2 | 2 | VH CDR2 | AIVGSGDSTYYADSVKG |
| 3 | 2 | VH CDR3 | AKDYSSGDWIDYGMDV |
| 4 | 2 | VL CDR1 | QASQDISNYLN |
| 5 | 2 | VL CDR2 | DASNLAT |
| 6 | 2 | VL CDR3 | QQFDLLPPT |
| 7 | 2 | VH FR1 | EVQLVESGGGLVKPGGSLRLSCAASG |
| 8 | 2 | VH FR2 | WVRQAPGKGLEWVS |
| 9 | 2 | VH FR3 | RFTISRDNSKNTLYLQMNSLRAEDTAVYYC |
| 10 | 2 | VH FR4 | WGQGTTVTVSS |
| 11 | 2 | VH DNA | GAGGTGCAGCTGGTGGAGTCTGGGGGAGGCCTGGTCAAGCCTG |
| | | | GGGGGTCCCTGAGACTCTCCTGTGCAGCCTCTGGATTCACCTTC |
| | | | AGTGAATATACCATGAACTGGGTCCGCCAGGCTCCAGGGAAGG |
| | | | GGCTGGAGTGGGTCTCAGCTATTGTAGGTAGTGGTGACAGCAC |
| | | | ATACTACGCAGACTCCGTGAAGGGCCGGTTCACCATCTCCAGA |
| | | | GACAATTCCAAGAACACGCTGTATCTGCAAATGAACAGCCTGA |
| | | | GAGCCGAGGACACGGCGGTGTACTACTGCGCCAAGGACTACAG |
| | | | CTCCGGAGACTGGATCGATTATGGAATGGACGTATGGGGCCAG |
| | | | GGAACAACTGTCACCGTCTCCTCA |
| 12 | 2 | VH Protein | EVQLVESGGGLVKPGGSLRLSCAASGFTFSEYTMNWVRQAPGKG |
| | | | LEWVSAIVGSGDSTYYADSVKGRFTISRDNSKNTLYLQMNSLRAE |
| | | | DTAVYYCAKDYSSGDWIDYGMDVWGQGTTVTVSS |
| 13 | 2 | VL FR1 | DIQMTQSPSSLSASVGDRVTITC |
| 14 | 2 | VL FR2 | WYQQKPGKAPKLLIY |
| 15 | 2 | VL FR3 | GVPSRFSGSGSGTDFTFTISSLQPEDIATYYC |
| 16 | 2 | VL FR4 | FGGGTKVEIK |
| 17 | 2 | VL DNA | GACATCCAGATGACCCAGTCTCCATCCTCCCTGTCTGCATCTGT |
| | | | AGGAGACAGAGTCACCATCACTTGCCAGGCGAGTCAGGACATT |
| | | | AGCAACTATTTAAATTGGTATCAGCAGAAACCAGGGAAAGCCC |
| | | | CTAAGCTCCTGATCTACGATGCATCCAATTTGGCAACAGGGGTC |
| | | | CCATCAAGGTTCAGTGGAAGTGGATCTGGGACAGATTTTACTTT |
| | | | CACCATCAGCAGCCTGCAGCCTGAAGATATTGCAACATATTAC |
| | | | TGTCAGCAGTTCGATCTCCTCCCTCCTACTTTTGGCGGAGGGAC |
| | | | CAAGGTTGAGATCAAA |
| 18 | 2 | VL Protein | DIQMTQSPSSLSASVGDRVTITCQASQDISNYLNWYQQKPGKAPK |
| | | | LLIYDASNLATGVPSRFSGSGSGTDFTFTISSLQPEDIATYYCQQFD |
| | | | LLPPTFGGGTKVEIK |

19-100: Not used

| | | | |
|---|---|---|---|
| 101 | 5 | VH CDR1 | FTFSDYAMI |
| 102 | 5 | VH CDR2 | AISGGGESTYYADSVKG |
| 103 | 5 | VH CDR3 | AKDYSSGDWIDYGMDV |
| 104 | 5 | VL CDR1 | QASQDISNYLN |
| 105 | 5 | VL CDR2 | DASNLAT |
| 106 | 5 | VL CDR3 | QQFDLLPPT |
| 107 | 5 | VH FR1 | EVQLLESGGGLVQPGGSLRLSCAASG |
| 108 | 5 | VH FR2 | WVRQAPGKGLEWVS |
| 109 | 5 | VH FR3 | RFTISRDNSKNTLYLQMNSLRAEDTAVYYC |
| 110 | 5 | VH FR4 | WGQGTTVTVSS |
| 111 | 5 | VH DNA | GAGGTGCAGCTGTTGGAGTCTGGGGGAGGCTTGGTACAGCCTG |
| | | | GGGGGTCCCTGAGACTCTCCTGTGCAGCCTCTGGATTCACCTTT |
| | | | AGCGACTATGCCATGATATGGGTCCGCCAGGCTCCAGGGAAGG |
| | | | GGCTGGAGTGGGTCTCAGCTATTAGTGGTGGAGGTGAAAGCAC |
| | | | ATACTACGCAGACTCCGTGAAGGGCCGGTTCACCATCTCCAGA |
| | | | GACAATTCCAAGAACACGCTGTATCTGCAAATGAACAGCCTGA |
| | | | GAGCCGAGGACACGGCGGTGTACTACTGCGCCAAGGACTACAG |
| | | | CTCCGGAGACTGGATCGATTATGGAATGGACGTATGGGGCCAG |
| | | | GGAACAACTGTCACCGTCTCCTCA |
| 112 | 5 | VH Protein | EVQLLESGGGLVQPGGSLRLSCAASGFTFSDYAMIWVRQAPGKGL |
| | | | EWVSAISGGGESTYYADSVKGRFTISRDNSKNTLYLQMNSLRAED |
| | | | TAVYYCAKDYSSGDWIDYGMDVWGQGTTVTVSS |
| 113 | 5 | VL FR1 | DIQMTQSPSSLSASVGDRVTITC |
| 114 | 5 | VL FR2 | WYQQKPGKAPKLLIY |
| 115 | 5 | VL FR3 | GVPSRFSGSGSGTDFTFTISSLQPEDIATYYC |
| 116 | 5 | VL FR4 | FGGGTKVEIK |
| 117 | 5 | VL DNA | GACATCCAGATGACCCAGTCTCCATCCTCCCTGTCTGCATCTGT |
| | | | AGGAGACAGAGTCACCATCACTTGCCAGGCGAGTCAGGACATT |
| | | | AGCAACTATTTAAATTGGTATCAGCAGAAACCAGGGAAAGCCC |
| | | | CTAAGCTCCTGATCTACGATGCATCCAATTTGGCAACAGGGGTC |
| | | | CCATCAAGGTTCAGTGGAAGTGGATCTGGGACAGATTTTACTTT |
| | | | CACCATCAGCAGCCTGCAGCCTGAAGATATTGCAACATATTAC |
| | | | TGTCAGCAGTTCGATCTCCTCCCTCCTACTTTTGGCGGAGGGAC |
| | | | CAAGGTTGAGATCAAA |
| 118 | 5 | VL Protein | DIQMTQSPSSLSASVGDRVTITCQASQDISNYLNWYQQKPGKAPK |
| | | | LLIYDASNLATGVPSRFSGSGSGTDFTFTISSLQPEDIATYYCQQFD |
| | | | LLPPTEGGGTKVEIK |

119-200: Not used

| | | | |
|---|---|---|---|
| 201 | 44 | VH CDR1 | GTFDNYYIS |
| 202 | 44 | VH CDR2 | GIFPIFGTANYAQKFQG |
| 203 | 44 | VH CDR3 | AREVGHYSGSPYYMDV |
| 204 | 44 | VL CDR1 | RASQSINSWLA |
| 205 | 44 | VL CDR2 | DASSLES |
| 206 | 44 | VL CDR3 | QQVGPYLT |
| 207 | 44 | VH FR1 | QVQLVQSGAEVKKPGSSVKVSCKASG |
| 208 | 44 | VH FR2 | WVRQAPGQGLEWMG |
| 209 | 44 | VH FR3 | RVTITADESTSTAYMELSSLRSEDTAVYYC |
| 210 | 44 | VH FR4 | WGKGTTVTVSS |
| 211 | 44 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | GACAACTATTACATCAGCTGGGTGCGACAGGCCCCTGGACAAG |
| | | | GGCTTGAGTGGATGGGAGGGATCTTCCCTATCTTCGGTACCGCA |
| | | | AACTACGCACAGAAGTTCCAGGGCAGAGTCACGATTACCGCGG |
| | | | ACGAATCCACGAGCACAGCCTACATGGAGCTGAGCAGCCTGAG |
| | | | ATCTGAGGACACGGCGGTGTACTACTGCGCCAGAGAAGTCGGA |
| | | | CACTACTCCGGCAGCCCATACTACATGGACGTATGGGGCAAGG |
| | | | GTACAACTGTCACCGTCTCCTCA |
| 212 | 44 | VH Protein | QVQLVQSGAEVKKPGSSVKVSCKASGGTFDNYYISWVRQAPGQG |
| | | | LEWMGGIFPIFGTANYAQKFQGRVTITADESTSTAYMELSSLRSED |
| | | | TAVYYCAREVGHYSGSPYYMDVWGKGTTVTVSS |
| 213 | 44 | VL FR1 | DIQMTQSPSTLSASVGDRVTITC |
| 214 | 44 | VL FR2 | WYQQKPGKAPKLLIS |
| 215 | 44 | VL FR3 | GVPSRFSGSGSGTEFTLTTSSLQPDDFATYYC |
| 216 | 44 | VL FR4 | FGGGTKVEIK |
| 217 | 44 | VL DNA | GACATCCAGATGACCCAGTCTCCTTCCACCCTGTCTGCATCTGT |
| | | | AGGAGACAGAGTCACCATCACTTGCCGGGCCAGTCAGAGTATT |
| | | | AATAGCTGGTTGGCCTGGTATCAGCAGAAACCAGGGAAAGCCC |
| | | | CTAAGCTCCTGATCTCCGATGCCTCCAGTTTGGAAAGTGGGGTC |
| | | | CCATCAAGGTTCAGCGGCAGTGGATCTGGGACAGAATTCACTC |
| | | | TCACCATCAGCAGCCTGCAGCCTGATGATTTTGCAACTTATTAC |
| | | | TGCCAGCAGGTCGGCCCCTACCTCACTTTTGGCGGAGGGACCA |
| | | | AGGTTGAGATCAAA |
| 218 | 44 | VL Protein | DIQMTQSPSTLSASVGDRVTITCRASQSINSWLAWYQQKPGKAPK |
| | | | LLISDASSLESGVPSRFSGSGSGTEFTLTISSLQPDDFATYYCQQVG |
| | | | PYLTFGGGTKVEIK |

219-300: Not used

| | | | |
|---|---|---|---|
| 301 | 58 | VH CDR1 | FTFGDYAMS |
| 302 | 58 | VH CDR2 | FIGSKFYGGETEYTASVKG |
| 303 | 58 | VH CDR3 | ARGPRRYTYGMDV |
| 304 | 58 | VL CDR1 | RASQSISSYLN |
| 305 | 58 | VL CDR2 | AASSLQS |
| 306 | 58 | VL CDR3 | QQSSTPLT |
| 307 | 58 | VH FR1 | EVQLVESGGGLVQPGRSLRLSCTASG |
| 308 | 58 | VH FR2 | WFRQAPGKGLEWVG |
| 309 | 58 | VH FR3 | RFTISRDGSKSIAYLQMNSLKTEDTAVYYC |
| 310 | 58 | VH FR4 | WGQGTTVTVSS |
| 311 | 58 | VH DNA | GAGGTGCAGCTGGTGGAGTCTGGGGGAGGCTTGGTACAGCCAG |
| | | | GGCGGTCCCTGAGACTCTCCTGTACAGCTTCTGGATTCACCTTT |
| | | | GGTGATTATGCTATGAGCTGGTTCCGCCAGGCTCCAGGGAAGG |
| | | | GGCTGGAGTGGGTAGGTTTCATTGGAAGCAAATTCTATGGTGG |
| | | | GGAAACAGAATACACCGCGTCTGTGAAAGGCAGATTCACCATC |
| | | | TCAAGAGATGGTTCCAAAAGCATCGCCTATCTGCAAATGAACA |
| | | | GCCTGAAAACCGAGGACACGGCGGTGTACTACTGCGCCAGAGG |
| | | | ACCAAGACGCTACACATACGGAATGGACGTATGGGGCCAGGG |
| | | | AACAACTGTCACCGTCTCCTCA |
| 312 | 58 | VH Protein | EVQLVESGGGLVQPGRSLRLSCTASGFTFGDYAMSWFRQAPGKG |
| | | | LEWVGFIGSKFYGGETEYTASVKGRFTISRDGSKSIAYLQMNSLKT |
| | | | EDTAVYYCARGPRRYTYGMDVVVGQGTTVTVSS |
| 313 | 58 | VL FR1 | DIQMTQSPSSLSASVGDRVTITC |
| 314 | 58 | VL FR2 | WYQQKPGKAPKLLIY |
| 315 | 58 | VL FR3 | GVPSRFSGSGSGTDFTLTISSLQPEDFATYYC |
| 316 | 58 | VL FR4 | FGGGTKVEIK |
| 317 | 58 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGCTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAAGCTCCACCCCCCTCACTTTTGGCGGAGGGACCAA |
| | | | GGTTGAGATCAAA |
| 318 | 58 | VL Protein | DIQMTQSPSSLSASVGDRVTITCRASQSISSYLNWYQQKPGKAPKL |
| | | | LIYAASSLQSGVPSRFSGSGSGTDFTLTISSLQPEDFATYYCQQSSTP |
| | | | LTFGGGTKVEIK |

319-400: Not used

| | | | |
|---|---|---|---|
| 401 | 10 | VH CDR1 | FTFDDYAVH |
| 402 | 10 | VH CDR2 | GISWSSGLIGYADSVKG |
| 403 | 10 | VH CDR3 | AKGPPTYQDYFDL |
| 404 | 10 | VL CDR1 | RASQSVSRYLA |
| 405 | 10 | VL CDR2 | DASNRAT |
| 406 | 10 | VL CDR3 | QQVSFFPPIT |
| 407 | 10 | VH FR1 | EVQLVESGGGLVQPGRSLRLSCAASG |
| 408 | 10 | VH FR2 | WVRQAPGKGLEWVS |
| 409 | 10 | VH FR3 | RFTISRDNAKNSLYLQMNSLRAEDTAVYYC |
| 410 | 10 | VH FR4 | WGRGTLVTVSS |
| 411 | 10 | VH DNA | GAAGTGCAGCTGGTGGAGTCTGGGGGAGGCTTGGTACAGCCTG |
| | | | GCAGGTCCCTGAGACTCTCCTGTGCAGCCTCTGGATTCACCTTT |
| | | | GATGATTATGCCGTGCACTGGGTCCGGCAAGCTCCAGGGAAGG |
| | | | GCCTGGAGTGGGTCTCAGGTATTAGTTGGAGTAGTGGACTAAT |
| | | | AGGCTATGCGGACTCTGTGAAGGGCCGATTCACCATCTCCAGA |
| | | | GACAACGCCAAGAACTCCCTGTATCTGCAAATGAACAGTCTGA |
| | | | GAGCTGAGGACACGGCGGTGTACTACTGCGCCAAGGGCCCTCC |
| | | | TACCTACCAAGACTACTTCGACCTATGGGGGAGAGGTACCTTG |
| | | | GTCACCGTCTCCTCA |
| 412 | 10 | VH Protein | EVQLVESGGGLVQPGRSLRLSCAASGFTFDDYAVHWVRQAPGKG |
| | | | LEWVSGISWSSGLIGYADSVKGRFTISRDNAKNSLYLQMNSLRAE |
| | | | DTAVYYCAKGPPTYQDYFDLWGRGTLVTVSS |
| 413 | 10 | VL FR1 | EIVLTQSPATLSLSPGERATLSC |
| 414 | 10 | VL FR2 | WYQQKPGQAPRLLIY |
| 415 | 10 | VL FR3 | GIPARFSGSGSGTDFTLTISSLEPEDFAVYYC |
| 416 | 10 | VL FR4 | FGGGTKVEIK |
| 417 | 10 | VL DNA | GAAATTGTGTTGACACAGTCTCCAGCCACCCTGTCTTTGTCTCC |
| | | | AGGGGAAAGAGCCACCCTCTCCTGCAGGGCCAGTCAGAGTGTT |
| | | | AGCAGGTACTTAGCCTGGTACCAACAGAAACCTGGCCAGGCTC |
| | | | CCAGGCTCCTCATCTATGATGCATCCAACAGGGCCACTGGCATC |
| | | | CCAGCCAGGTTCAGTGGCAGTGGGTCTGGGACAGACTTCACTC |
| | | | TCACCATCAGCAGCCTAGAGCCTGAAGATTTTGCAGTTTATTAC |
| | | | TGTCAGCAGGTCAGTTTCTTCCCTCCTATCACTTTTGGCGGAGG |
| | | | GACCAAGGTTGAGATCAAA |
| 418 | 10 | VL Protein | EIVLTQSPATLSLSPGERATLSCRASQSVSRYLAWYQQKPGQAPRL |
| | | | LIYDASNRATGIPARFSGSGSGTDFTLTISSLEPEDFAVYYCQQVSF |
| | | | FPPITFGGGTKVEIK |

419-500: Not used

| | | | |
|---|---|---|---|
| 501 | 38 | VH CDR1 | FTFSGHLMS |
| 502 | 38 | VH CDR2 | AISGSAGETYYADSVKG |
| 503 | 38 | VH CDR3 | ARDAYYDDWSGWADWYFDL |
| 504 | 38 | VL CDR1 | RASQSVSRYLA |
| 505 | 38 | VL CDR2 | DASNRAT |
| 506 | 38 | VL CDR3 | QQVSLLPPT |
| 507 | 38 | VH FR1 | EVQLLESGGGLVQPGGSLRLSCAASG |
| 508 | 38 | VH FR2 | WVRQAPGKGLEWVS |
| 509 | 38 | VH FR3 | RFTISRDNSKNTLYLQMNSLRAEDTAVYYC |
| 510 | 38 | VH FR4 | WGRGTLVTVSS |
| 511 | 38 | VH DNA | GAGGTGCAGCTGTTGGAGTCTGGGGGAGGCTTGGTACAGCCTG |
| | | | GGGGGTCCCTGAGACTCTCCTGTGCAGCCTCTGGATTCACCTTT |
| | | | AGCGGACACCTAATGAGCTGGGTCCGCCAGGCTCCAGGGAAGG |
| | | | GGCTGGAGTGGGTCTCAGCTATTAGTGGATCCGCAGGTGAAAC |
| | | | ATACTACGCAGACTCCGTGAAGGGCCGGTTCACCATCTCCAGA |
| | | | GACAATTCCAAGAACACGCTGTATCTGCAAATGAACAGCCTGA |
| | | | GAGCCGAGGACACGGCGGTGTACTACTGCGCCAGAGATGCGTA |
| | | | CTACGACGACTGGAGCGGATGGGCCGATTGGTACTTCGATTTA |
| | | | TGGGGGAGAGGTACCTTGGTCACCGTCTCCTCA |
| 512 | 38 | VH Protein | EVQLLESGGGLVQPGGSLRLSCAASGFTFSGHLMSWVRQAPGKG |
| | | | LEWVSATSGSAGETYVADSVKGRFTTSRDNSKNTLYLQMNSLRAE |
| | | | DTAVYYCARDAYYDDWSGWADWYFDLWGRGTLVTVSS |
| 513 | 38 | VL FR1 | EIVLTQSPATLSLSPGERATLSC |
| 514 | 38 | VL FR2 | WYQQKPGQAPRLLIY |
| 515 | 38 | VL FR3 | GIPARFSGSGSGTDFTLTISSLEPEDFAVYYC |
| 516 | 38 | VL FR4 | FGGGTKVEIK |
| 517 | 38 | VL DNA | GAAATTGTGTTGACACAGTCTCCAGCCACCCTGTCTTTGTCTCC |
| | | | AGGGGAAAGAGCCACCCTCTCCTGCAGGGCCAGTCAGAGTGTT |
| | | | AGCAGGTACTTAGCCTGGTACCAACAGAAACCTGGCCAGGCTC |
| | | | CCAGGCTCCTCATCTATGATGCATCCAACAGGGCCACTGGCATC |
| | | | CCAGCCAGGTTCAGTGGCAGTGGGTCTGGGACAGACTTCACTC |
| | | | TCACCATCAGCAGCCTAGAGCCTGAAGATTTTGCAGTTTATTAC |
| | | | TGTCAGCAGGTCAGTCTCCTCCCTCCTACTTTTGGCGGAGGGAC |
| | | | CAAGGTTGAGATCAAA |
| 518 | 38 | VL Protein | EIVLTQSPATLSLSPGERATLSCRASQSVSRYLAWYQQKPGQAPRL |
| | | | LIYDASNRATGIPARFSGSGSGTDFTLTISSLEPEDFAVYYCQQVSL |
| | | | LPPTFGGGTKVEIK |

519-600: Not used

| | | | |
|---|---|---|---|
| 601 | 15 | VH CDR1 | FTFGDVAMS |
| 602 | 15 | VH CDR2 | YIGSKAYGGETEYTASVKG |
| 603 | 15 | VH CDR3 | ARAGHSYGSIASNWFDP |
| 604 | 15 | VL CDR1 | RASQSISSYLN |
| 605 | 15 | VL CDR2 | GASSLQS |
| 606 | 15 | VL CDR3 | QQGFYTPWT |
| 607 | 15 | VH FR1 | EVQLVESGGGLVQPGRSLRLSCTASG |
| 608 | 15 | VH FR2 | WFRQAPGKGLEWVG |
| 609 | 15 | VH FR3 | RFTISRDGSKSIAYLQMNSLKTEDTAVYYC |
| 610 | 15 | VH FR4 | WGQGTLVTVSS |
| 611 | 15 | VH DNA | GAGGTGCAGCTGGTGGAGTCTGGGGGAGGCTTGGTACAGCCAG |
| | | | GGCGGTCCCTGAGACTCTCCTGTACAGCTTCTGGATTCACCTTT |
| | | | GGTGATGTCGCTATGTCCTGGTTCCGCCAGGCTCCAGGGAAGG |
| | | | GGCTGGAGTGGGTAGGTTACATTGGAAGCAAAGCTTATGGTGG |
| | | | GGAAACAGAATACACCGCGTCTGTGAAAGGCAGATTCACCATC |
| | | | TCAAGAGATGGTTCCAAAAGCATCGCCTATCTGCAAATGAACA |
| | | | GCCTGAAAACCGAGGACACGGCGGTGTACTACTGCGCCAGAGC |
| | | | TGGACACAGCTACGGATCCATCGCCAGCAACTGGTTCGACCCA |
| | | | TGGGGACAGGGTACATTGGTCACCGTCTCCTCA |
| 612 | 15 | VH Protein | EVQLVESGGGLVQPGRSLRLSCTASGFTFGDVAMSWFRQAPGKG |
| | | | LEWVGYIGSKAYGGETEYTASVKGRFTISRDGSKSIAYLQMNSLK |
| | | | TEDTAVYYCARAGHSYGSIASNWFDPWGQGTLVTVSS |
| 613 | 15 | VL FR1 | DIQMTQSPSSLSASVGDRVTITC |
| 614 | 15 | VL FR2 | WYQQKPGKAPKLLIY |
| 615 | 15 | VL FR3 | GVPSRFSGSGSGTDFTLTISSLQPEDFATYYC |
| 616 | 15 | VL FR4 | FGGGTKVEIK |
| 617 | 15 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGGTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAGGATTCTACACTCCTTGGACTTTTGGCGGAGGGAC |
| | | | CAAGGTTGAGATCAAA |
| 618 | 15 | VL Protein | DIQMTQSPSSLSASVGDRVTITCRASQSISSYLNWYQQKPGKAPKL |
| | | | LIYGASSLQSGVPSRFSGSGSGTDFTLTISSLQPEDFATYYCQQGFY |
| | | | TPWTFGGGTKVEIK |

619-700: Not used

| | | | |
|---|---|---|---|
| 701 | 35 | VH CDR1 | GTFSSAAIS |
| 702 | 35 | VH CDR2 | NIIPIVGIANYAQKFQG |
| 703 | 35 | VH CDR3 | ARDTGRGYTRHFWFDP |
| 704 | 35 | VL CDR1 | RASQSISSYLN |
| 705 | 35 | VL CDR2 | AASSLQS |
| 706 | 35 | VL CDR3 | QQSDILYT |
| 707 | 35 | VH FR1 | QVQLVQSGAEVKKPGSSVKVSCKASG |
| 708 | 35 | VH FR2 | WVRQAPGQGLEWMG |
| 709 | 35 | VH FR3 | RVTITADESTSTAYMELSSLRSEDTAVYYC |
| 710 | 35 | VH FR4 | WGQGTLVTVSS |
| 711 | 35 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | AGCTCCGCCGCTATCAGCTGGGTGCGACAGGCCCCTGGACAAG |
| | | | GGCTTGAGTGGATGGGAAACATCATCCCTATCGTAGGTATAGC |
| | | | AAACTACGCACAGAAGTTCCAGGGCAGAGTCACGATTACCGCG |
| | | | GACGAATCCACGAGCACAGCCTACATGGAGCTGAGCAGCCTGA |
| | | | GATCTGAGGACACGGCGGTGTACTACTGCGCCAGAGACACGGG |
| | | | ACGGGGATACACCAGACACTTCTGGTTTGACCCCTGGGGACAG |
| | | | GGTACATTGGTCACCGTCTCCTCA |
| 712 | 35 | VH Protein | QVQLVQSGAEVKKPGSSVKVSCKASGGTFSSAAISWVRQAPGQG |
| | | | LEWMGNIIPIVGIANYAQKFQGRVTITADESTSTAYMELSSLRSED |
| | | | TAVYYCARDTGRGYTRHFWFDPWGQGTLVTVSS |
| 713 | 35 | VL FR1 | DIQMTQSPSSLSASVGDRVTITC |
| 714 | 35 | VL FR2 | WYQQKPGKAPKLLIY |
| 715 | 35 | VL FR3 | GVPSRFSGSGSGTDFTLTISSLQPEDFATYYC |
| 716 | 35 | VL FR4 | FGGGTKVEIK |
| 717 | 35 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGCTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAAGCGACATCCTCTACACTTTTGGCGGAGGGACCAA |
| | | | GGTTGAGATCAAA |
| 718 | 35 | VL Protein | DIQMTQSPSSLSASVGDRVTITCRASQSISSYLNWYQQKPGKAPKL |
| | | | LIYAASSLQSGVPSRFSGSGSGTDFTLTISSLQPEDFATYYCQQSDIL |
| | | | YTFGGGTKVEIK |

719-800: Not used

| | | | |
|---|---|---|---|
| 801 | 47 | VH CDR1 | GTFSNYAIS |
| 802 | 47 | VH CDR2 | GIIPIFGTANYAQKFQG |
| 803 | 47 | VH CDR3 | ARGRGALALVGPYYGMDV |
| 804 | 47 | VL CDR1 | RSSQSLLHSNGYNYLD |
| 805 | 47 | VL CDR2 | LGSHRAS |
| 806 | 47 | VL CDR3 | MQALRAPT |
| 807 | 47 | VH FR1 | EVQLVQSGAEVKKPGSSVKVSCKASG |
| 808 | 47 | VH FR2 | WVRQAPGQGLEWMG |
| 809 | 47 | VH FR3 | RVTITADESTSTAYMELSSLRSEDTAVYYC |
| 810 | 47 | VH FR4 | WGQGTTVTVSS |
| 811 | 47 | VH DNA | GAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | AGCAACTATGCTATCAGCTGGGTGCGACAGGCCCCTGGACAAG |
| | | | GGCTTGAGTGGATGGGAGGGATCATCCCTATCTTTGGTACAGC |
| | | | AAACTACGCACAGAAGTTCCAGGGCAGAGTCACGATTACCGCG |
| | | | GACGAATCCACGAGCACAGCCTACATGGAGCTGAGCAGCCTGA |
| | | | GATCTGAGGACACGGCGGTGTACTACTGCGCCAGAGGCAGAGG |
| | | | CGCTCTGGCACTCGTCGGACCATACTACGGAATGGACGTATGG |
| | | | GGCCAGGGAACAACTGTCACCGTCTCCTCA |
| 812 | 47 | VH Protein | EVQLVQSGAEVKKPGSSVKVSCKASGGTFSNYAISWVRQAPGQG |
| | | | LEWMGGIIPIFGTANYAQKFQGRVTITADESTSTAYMELSSLRSED |
| | | | TAVYYCARGRGALALVGPYYGMDVWGQGTTVTVSS |
| 813 | 47 | VL FR1 | DIVMTQSPLSLPVTPGEPASISC |
| 814 | 47 | VL FR2 | WYLQKPGQSPQLLIY |
| 815 | 47 | VL FR3 | GVPDRFSGSGSGTDFTLKISRVEAEDVGVYYC |
| 816 | 47 | VL FR4 | FGGGTKVEIK |
| 817 | 47 | VL DNA | GATATTGTGATGACTCAGTCTCCACTCTCCCTGCCCGTCACCCC |
| | | | TGGAGAGCCGGCCTCCATCTCCTGCAGGTCTAGTCAGAGCCTCC |
| | | | TGCATAGTAATGGATACAACTATTTGGATTGGTACCTGCAGAA |
| | | | GCCAGGGCAGTCTCCACAGCTCCTGATCTATTTGGGTTCTCATC |
| | | | GGGCCTCCGGGGTCCCTGACAGGTTCAGTGGCAGTGGATCAGG |
| | | | CACAGATTTTACACTGAAAATCAGCAGAGTGGAGGCTGAGGAT |
| | | | GTTGGGGTTTATTACTGCATGCAGGCACTCCGAGCCCCTACTTT |
| | | | TGGCGGAGGGACCAAGGTTGAGATCAAA |
| 818 | 47 | VL Protein | DIVMTQSPLSLPVTPGEPASISCRSSQSLLHSNGYNYLDWYLQKPG |
| | | | QSPQLLTYLGSHRASGVPDRFSGSGSGTDFTLKTSRVEAEDVGVYY |
| | | | CMQALRAPTFGGGTKVEIK |

819-900 not used

| | | | |
|---|---|---|---|
| 901 | 46 | VH CDR1 | FTFGDYAMS |
| 902 | 46 | VH CDR2 | FIGSKAYGGTTEYTASVKG |
| 903 | 46 | VH CDR3 | ARGPRRYTYGMDV |
| 904 | 46 | VL CDR1 | RASQSISSYLN |
| 905 | 46 | VL CDR2 | AASSLQS |
| 906 | 46 | VL CDR3 | QQSSTPLT |
| 907 | 46 | VH FR1 | EVQLVESGGGLVQPGRSLRLSCTASG |
| 908 | 46 | VH FR2 | WFRQAPGKGLEWVG |
| 909 | 46 | VH FR3 | RFTISRDGSKSIAYLQMNSLKTEDTAVYYC |
| 910 | 46 | VH FR4 | WGQGTTVTVSS |
| 911 | 46 | VH DNA | GAGGTGCAGCTGGTGGAGTCTGGGGGAGGCTTGGTACAGCCAG |
| | | | GGCGGTCCCTGAGACTCTCCTGTACAGCTTCTGGATTCACCTTT |
| | | | GGTGATTATGCTATGAGCTGGTTCCGCCAGGCTCCAGGGAAGG |
| | | | GGCTGGAGTGGGTAGGTTTCATTGGAAGCAAAGCTTATGGTGG |
| | | | GACAACAGAATACACCGCGTCTGTGAAAGGCAGATTCACCATC |
| | | | TCAAGAGATGGTTCCAAAAGCATCGCCTATCTGCAAATGAACA |
| | | | GCCTGAAAACCGAGGACACGGCGGTGTACTACTGCGCCAGAGG |
| | | | ACCAAGACGCTACACATACGGAATGGACGTATGGGGCCAGGG |
| | | | AACAACTGTCACCGTCTCCTCA |
| 912 | 46 | VH Protein | EVQLVESGGGLVQPGRSLRLSCTASGFTFGDYAMSWFRQAPGKG |
| | | | LEWVGFIGSKAYGGTTEYTASVKGRFTISRDGSKSIAYLQMNSLKT |
| | | | EDTAVYYCARGPRRYTYGMDVWGQGTTVTVSS |
| 913 | 46 | VL FR1 | DIQMTQSPSSLSASVGDRVTITC |
| 914 | 46 | VL FR2 | WYQQKPGKAPKLLIY |
| 915 | 46 | VL FR3 | GVPSRFSGSGSGTDFTLTISSLQPEDFATYYC |
| 916 | 46 | VL FR4 | FGGGTKVEIK |
| 917 | 46 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGCTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAAGCTCCACCCCCCTCACTTTTGGCGGAGGGACCAA |
| | | | GGTTGAGATCAAA |
| 918 | 46 | VL Protein | DIQMTQSPSSLSASVGDRVTITCRASQSISSYLNWYQQKPGKAPKL |
| | | | LIYAASSLQSGVPSRFSGSGSGTDFTLTISSLQPEDFATYYCQQSSTP |
| | | | LTFGGGTKVEIK |

919-1000 not used

| | | | |
|---|---|---|---|
| 1001 | 32 | VH CDR1 | GTFSSYAIS |
| 1002 | 32 | VH CDR2 | GIIPISGTANYAQKFQG |
| 1003 | 32 | VH CDR3 | ARDTGRGYTRHFWFDP |
| 1004 | 32 | VL CDR1 | RASQSISSYLN |
| 1005 | 32 | VL CDR2 | AASSLQS |
| 1006 | 32 | VL CDR3 | QQSDILYT |
| 1007 | 32 | VH FR1 | QVQLVQSGAEVKKPGSSVKVSCKASG |
| 1008 | 32 | VH FR2 | WVRQAPGQGLEWMG |
| 1009 | 32 | VH FR3 | RVTITADESTSTAYMELSSLRSEDTAVYYC |
| 1010 | 32 | VH FR4 | WGQGTLVTVSS |
| 1011 | 32 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | AGCAGCTATGCTATCAGCTGGGTGCGACAGGCCCCTGGACAAG |
| | | | GGCTTGAGTGGATGGGAGGGATCATCCCTATCTCTGGTACAGC |
| | | | AAACTACGCACAGAAGTTCCAGGGCAGAGTCACGATTACCGCG |
| | | | GACGAATCCACGAGCACAGCCTACATGGAGCTGAGCAGCCTGA |
| | | | GATCTGAGGACACGGCGGTGTACTACTGCGCCAGAGACACGGG |
| | | | ACGGGGATACACCAGACACTTCTGGTTTGACCCCTGGGGACAG |
| | | | GGTACATTGGTCACCGTCTCCTCA |
| 1012 | 32 | VH Protein | QVQLVQSGAEVKKPGSSVKVSCKASGGTFSSYAISWVRQAPGQG |
| | | | LEWMGGIIPISGTANYAQKFQGRVTITADESTSTAYMELSSLRSED |
| | | | TAVYYCARDTGRGYTRHFWFDPWGQGTLVTVSS |
| 1013 | 32 | VL FR1 | DIQMTQSPSSLSASVGDRVTITC |
| 1014 | 32 | VL FR2 | WYQQKPGKAPKLLIY |
| 1015 | 32 | VL FR3 | GVPSRFSGSGSGTDFTLTISSLQPEDFATYYC |
| 1016 | 32 | VL FR4 | FGGGTKVEIK |
| 1017 | 32 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGCTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAAGCGACATCCTCTACACTTTTGGCGGAGGGACCAA |
| | | | GGTTGAGATCAAA |
| 1018 | 32 | VL Protein | DIQMTQSPSSLSASVGDRVTITCRASQSISSYLNWYQQKPGKAPKL |
| | | | LIYAASSLQSGVPSRFSGSGSGTDFTLTISSLQPEDFATYYCQQSDIL |
| | | | YTFGGGTKVEIK |

1019-2000 not used

| | | | |
|---|---|---|---|
| 2001 | 33 | VH CDR1 | GTFGNYAIS |
| 2002 | 33 | VH CDR2 | GIIPIPGIANYAQKFQG |
| 2003 | 33 | VH CDR3 | ARDTGRGYTRHFWFDP |
| 2004 | 33 | VL CDR1 | RASQSISSYLN |
| 2005 | 33 | VL CDR2 | AASSLQS |
| 2006 | 33 | VL CDR3 | QQSDILYT |
| 2007 | 33 | VH FR1 | QVQLVQSGAEVKKPGSSVKVSCKASG |
| 2008 | 33 | VH FR2 | WVRQAPGQGLEWMG |
| 2009 | 33 | VH FR3 | RVTITADESTSTAYMELSSLRSEDTAVYYC |
| 2010 | 33 | VH FR4 | WGQGTLVTVSS |
| 2011 | 33 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | GGAAACTATGCTATCAGCTGGGTGCGACAGGCCCCTGGACAAG |
| | | | GGCTTGAGTGGATGGGAGGGATCATCCCTATCCCAGGTATCGC |
| | | | AAACTACGCACAGAAGTTCCAGGGCAGAGTCACGATTACCGCG |
| | | | GACGAATCCACGAGCACAGCCTACATGGAGCTGAGCAGCCTGA |
| | | | GATCTGAGGACACGGCGGTGTACTACTGCGCCAGAGACACGGG |
| | | | ACGGGGATACACCAGACACTTCTGGTTTGACCCCTGGGGACAG |
| | | | GGTACATTGGTCACCGTCTCCTCA |
| 2012 | 33 | VH Protein | QVQLVQSGAEVKKPGSSVKVSCKASGGTFGNYAISWVRQAPGQG |
| | | | LEWMGGIIPIPGIANYAQKFQGRVTITADESTSTAYMELSSLRSEDT |
| | | | AVYYCARDTGRGYTRHFWFDPWGQGTLVTVSS |
| 2013 | 33 | VL FR1 | DIQMTQSPSSLSASVGDRVTITC |
| 2014 | 33 | VL FR2 | WYQQKPGKAPKLLIY |
| 2015 | 33 | VL FR3 | GVPSRFSGSGSGTDFTLTTSSLQPEDFATYYC |
| 2016 | 33 | VL FR4 | FGGGTKVEIK |
| 2017 | 33 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGCTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAAGCGACATCCTCTACACTTTTGGCGGAGGGACCAA |
| | | | GGTTGAGATCAAA |
| 2018 | 33 | VL Protein | DIQMTQSPSSLSASVGDRVTITCRASQSISSYLNWYQQKPGKAPKL |
| | | | LIYAASSLQSGVPSRFSGSGSGTDFTLTISSLQPEDFATYYCQQSDIL |
| | | | YTFGGGTKVEIK |

2019-3000 not used

| | | | |
|---|---|---|---|
| 3001 | 34 | VH CDR1 | GTFSSAAIS |
| 3002 | 34 | VH CDR2 | GIFPISGHANYAQKFQG |
| 3003 | 34 | VH CDR3 | ARDTGRGYTRHFWFDP |
| 3004 | 34 | VL CDR1 | RASQSISSYLN |
| 3005 | 34 | VL CDR2 | AASSLQS |
| 3006 | 34 | VL CDR3 | QQSDILYT |
| 3007 | 34 | VH FR1 | QVQLVQSGAEVKKPGSSVKVSCKASG |
| 3008 | 34 | VH FR2 | WVRQAPGQGLEWMG |
| 3009 | 34 | VH FR3 | RVTITADESTSTAYMELSSLRSEDTAVYYC |
| 3010 | 34 | VH FR4 | WGQGTLVTVSS |
| 3011 | 34 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | AGCAGCGCCGCTATCAGCTGGGTGCGACAGGCCCCTGGACAAG |
| | | | GGCTCGAGTGGATGGGAGGGATCTTCCCTATCTCCGGTCACGC |
| | | | AAACTACGCACAGAAGTTCCAGGGCAGAGTCACGATTACCGCG |
| | | | GACGAATCCACGAGCACAGCCTACATGGAGCTGAGCAGCCTGA |
| | | | GATCTGAGGACACGGCGGTGTACTACTGCGCCAGAGACACGGG |
| | | | ACGGGGATACACCAGACACTTCTGGTTTGACCCCTGGGGACAG |
| | | | GGTACATTGGTCACCGTCTCCTCA |
| 3012 | 34 | VH Protein | QVQLVQSGAEVKKPGSSVKVSCKASGGTFSSAAISWVRQAPGQG |
| | | | LEWMGGIFPISGHANYAQKFQGRVTITADESTSTAYMELSSLRSED |
| | | | TAVYYCARDTGRGYTRHFWFDPWGQGTLVTVSS |
| 3013 | 34 | VL FR1 | DIQMTQSPSSLSASVGDRVTITC |
| 3014 | 34 | VL FR2 | WYQQKPGKAPKLLIY |
| 3015 | 34 | VL FR3 | GVPSRFSGSGSGTDFTLTISSLQPEDFATYYC |
| 3016 | 34 | VL FR4 | FGGGTKVEIK |
| 3017 | 34 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGCTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAAGCGACATCCTCTACACTTTTGGCGGAGGGACCAA |
| | | | GGTTGAGATCAAA |
| 3018 | 34 | VL Protein | DIQMTQSPSSLSASVGDRVTITCRASQSISSYLNWYQQKPGKAPKL |
| | | | LIYAASSLQSGVPSRFSGSGSGTDFTLTISSLQPEDFATYYCQQSDIL |
| | | | YTFGGGTKVEIK |

3019-4000 not used

| | | | |
|---|---|---|---|
| 4001 | 36 | VH CDR1 | GTFATYAIS |
| 4002 | 36 | VH CDR2 | GIFPLSGTANYAQKFQG |
| 4003 | 36 | VH CDR3 | ARDTGRGYTRHFWFDP |
| 4004 | 36 | VL CDR1 | RASQSISSYLN |
| 4005 | 36 | VL CDR2 | AASSLQS |
| 4006 | 36 | VL CDR3 | QQSDILYT |
| 4007 | 36 | VH FR1 | QVQLVQSGAEVKKPGSSVKVSCKASG |
| 4008 | 36 | VH FR2 | WVRQAPGQGLEWMG |
| 4009 | 36 | VH FR3 | RVTITADESTSTAYMELSSLRSEDTAVYYC |
| 4010 | 36 | VH FR4 | WGQGTLVTVSS |
| 4011 | 36 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | GCAACCTATGCTATCAGCTGGGTGCGACAGGCCCCTGGACAAG |
| | | | GGCTTGAGTGGATGGGAGGGATCTTCCCTCTCTCCGGTACAGC |
| | | | AAACTACGCACAGAAGTTCCAGGGCAGAGTCACGATTACCGCG |
| | | | GACGAATCCACGAGCACAGCCTACATGGAGCTGAGCAGCCTGA |
| | | | GATCTGAGGACACGGCGGTGTACTACTGCGCCAGAGACACGGG |
| | | | ACGGGGATACACCAGACACTTCTGGTTTGACCCCTGGGGACAG |
| | | | GGTACATTGGTCACCGTCTCCTCA |
| 4012 | 36 | VH Protein | QVQLVQSGAEVKKPGSSVKVSCKASGGTFATYAISWVRQAPGQG |
| | | | LEWMGGIFPLSGTANYAQKFQGRVTITADESTSTAYMELSSLRSE |
| | | | DTAVYYCARDTGRGYTRHFWFDPWGQGTLVTVSS |
| 4013 | 36 | VL FR1 | DIQMTQSPSSLSASVGDRVTITC |
| 4014 | 36 | VL FR2 | WYQQKPGKAPKLLIY |
| 4015 | 36 | VL FR3 | GVPSRFSGSGSGTDFTLTISSLQPEDFATYYC |
| 4016 | 36 | VL FR4 | FGGGTKVEIK |
| 4017 | 36 | VL DNA | GACATCCAGATGACCCAGTCTCCATCCTCCCTGTCTGCATCTGT |
| | | | AGGAGACAGAGTCACCATCACTTGCCGGGCAAGTCAGAGCATT |
| | | | AGCAGCTATTTAAATTGGTATCAGCAGAAACCAGGGAAAGCCC |
| | | | CTAAGCTCCTGATCTATGCTGCATCCAGTTTGCAAAGTGGGGTC |
| | | | CCATCAAGGTTCAGTGGCAGTGGATCTGGGACAGATTTCACTCT |
| | | | CACCATCAGCAGTCTGCAACCTGAAGATTTTGCAACTTACTACT |
| | | | GTCAGCAAAGCGACATCCTCTACACTTTTGGCGGAGGGACCAA |
| | | | GGTTGAGATCAAA |
| 4018 | 36 | VL Protein | DIQMTQSPSSLSASVGDRVTITCRASQSISSYLNWYQQKPGKAPKL |
| | | | LIYAASSLQSGVPSRFSGSGSGTDFTLTISSLQPEDFATYYCQQSDIL |
| | | | YTFGGGTKVEIK |

1. An isolated antibody comprising:
i) (a) HCDR1 comprising the amino acid sequence of SEQ ID NO: 1; (b) HCDR2 comprising the amino acid sequence of SEQ ID NO: 2; (c) HCDR3 comprising the amino acid sequence of SEQ ID NO: 3; (d) LCDR1 comprising the amino acid sequence of SEQ ID NO: 4; (e) LCDR2 comprising the amino acid sequence of SEQ ID NO: 5; and (f) LCDR3 comprising the amino acid sequence of SEQ ID NO: 6; or
ii) (a) HCDR1 comprising the amino acid sequence of SEQ ID NO: 101; (b) HCDR2 comprising the amino acid sequence of SEQ ID NO: 102; (c) HCDR3 comprising the amino acid sequence of SEQ ID NO: 103; (d) LCDR1 comprising the amino acid sequence of SEQ ID NO: 104; (e) LCDR2 comprising the amino acid sequence of SEQ ID NO: 105; and (f) LCDR3 comprising the amino acid sequence of SEQ ID NO: 106; or
iii) (a) HCDR1 comprising the amino acid sequence of SEQ ID NO: 201; (b) HCDR2 comprising the amino acid sequence of SEQ ID NO: 202; (c) HCDR3 comprising the amino acid sequence of SEQ ID NO: 203; (d) LCDR1 comprising the amino acid sequence of SEQ ID NO: 204; (e) LCDR2 comprising the amino acid sequence of SEQ ID NO: 205; and (f) LCDR3 comprising the amino acid sequence of SEQ ID NO: 206; or
iv) (a) HCDR1 comprising the amino acid sequence of SEQ ID NO: 301; (b) HCDR2 comprising the amino acid sequence of SEQ ID NO: 302; (c) HCDR3 comprising the amino acid sequence of SEQ ID NO: 303; (d) LCDR1 comprising the amino acid sequence of SEQ ID NO: 304; (e) LCDR2 comprising the amino acid sequence of SEQ ID NO: 305; and (f) LCDR3 comprising the amino acid sequence of SEQ ID NO: 306; or
v) (a) HCDR1 comprising the amino acid sequence of SEQ ID NO: 401; (b) HCDR2 comprising the amino acid sequence of SEQ ID NO: 402; (c) HCDR3 comprising the amino acid sequence of SEQ ID NO: 403; (d) LCDR1 comprising the amino acid sequence of SEQ ID NO: 404; (e) LCDR2 comprising the amino acid sequence of SEQ ID NO: 405; and (f) LCDR3 comprising the amino acid sequence of SEQ ID NO: 406; or
vi) (a) HCDR1 comprising the amino acid sequence of SEQ ID NO: 501; (b) HCDR2 comprising the amino acid sequence of SEQ ID NO: 502; (c) HCDR3 comprising the amino acid sequence of SEQ ID NO: 503; (d) LCDR1 comprising the amino acid sequence of SEQ ID NO: 504; (e) LCDR2 comprising the amino acid sequence of SEQ ID NO: 505; and (f) LCDR3 comprising the amino acid sequence of SEQ ID NO: 506; or
vii) (a) HCDR1 comprising the amino acid sequence of SEQ ID NO: 601; (b) HCDR2 comprising the amino acid sequence of SEQ ID NO: 602; (c) HCDR3 comprising the amino acid sequence of SEQ ID NO: 603; (d) LCDR1 comprising the amino acid sequence of SEQ ID NO: 604; (e) LCDR2 comprising the amino acid sequence of SEQ ID NO: 605; and (f) LCDR3 comprising the amino acid sequence of SEQ ID NO: 606; or
viii) (a) HCDR1 comprising the amino acid sequence of SEQ ID NO: 701; (b) HCDR2 comprising the amino acid sequence of SEQ ID NO: 702; (c) HCDR3 comprising the amino acid sequence of SEQ ID NO: 703; (d) LCDR1 comprising the amino acid sequence of SEQ ID NO: 704; (e) LCDR2 comprising the amino acid sequence of SEQ ID NO: 705; and (f) LCDR3 comprising the amino acid sequence of SEQ ID NO: 706; or
ix) (a) HCDR1 comprising the amino acid sequence of SEQ ID NO: 801; (b) HCDR2 comprising the amino acid sequence of SEQ ID NO: 802; (c) HCDR3 comprising the amino acid sequence of SEQ ID NO: 803; (d) LCDR1 comprising the amino acid sequence of SEQ ID NO: 804; (e) LCDR2 comprising the amino acid sequence of SEQ ID NO: 805; and (f) LCDR3 comprising the amino acid sequence of SEQ ID NO: 806; or
x) (a) HCDR1 comprising the amino acid sequence of SEQ ID NO: 901; (b) HCDR2 comprising the amino acid sequence of SEQ ID NO: 902; (c) HCDR3 comprising the amino acid sequence of SEQ ID NO: 903; (d) LCDR1 comprising the amino acid sequence of SEQ ID NO: 904; (e) LCDR2 comprising the amino acid sequence of SEQ ID NO: 905; and (f) LCDR3 comprising the amino acid sequence of SEQ ID NO: 906; or
xi) (a) HCDR1 comprising the amino acid sequence of SEQ ID NO: 1001; (b) HCDR2 comprising the amino acid sequence of SEQ ID NO: 1002; (c) HCDR3 comprising the amino acid sequence of SEQ ID NO: 1003; (d) LCDR1 comprising the amino acid sequence of SEQ ID NO: 1004; (e) LCDR2 comprising the amino acid sequence of SEQ ID NO: 1005; and (f) LCDR3 comprising the amino acid sequence of SEQ ID NO: 1006; or
xii) (a) HCDR1 comprising the amino acid sequence of SEQ ID NO: 2001; (b) HCDR2 comprising the amino acid sequence of SEQ ID NO: 2002; (c) HCDR3 comprising the amino acid sequence of SEQ ID NO: 2003; (d) LCDR1 comprising the amino acid sequence of SEQ ID NO: 2004; (e) LCDR2 comprising the amino acid sequence of SEQ ID NO: 2005; and (f) LCDR3 comprising the amino acid sequence of SEQ ID NO: 2006; or
xiii) (a) HCDR1 comprising the amino acid sequence of SEQ ID NO: 3001; (b) HCDR2 comprising the amino acid sequence of SEQ ID NO: 3002; (c) HCDR3 comprising the amino acid sequence of SEQ ID NO: 3003; (d) LCDR1 comprising the amino acid sequence of SEQ ID NO: 3004; (e) LCDR2 comprising the amino acid sequence of SEQ ID NO: 3005; and (f) LCDR3 comprising the amino acid sequence of SEQ ID NO: 3006; or
xiv) (a) HCDR1 comprising the amino acid sequence of SEQ ID NO: 4001; (b) HCDR2 comprising the amino acid sequence of SEQ ID NO: 4002; (c) HCDR3 comprising the amino acid sequence of SEQ ID NO: 4003; (d) LCDR1 comprising the amino acid sequence of SEQ ID NO: 4004; (e) LCDR2 comprising the amino acid sequence of SEQ ID NO: 4005; and (f) LCDR3 comprising the amino acid sequence of SEQ ID NO: 4006.

2. The isolated antibody of claim 1, wherein the antibody comprises a heavy chain variable region (VH) and a light chain variable region (VL), wherein:
i) the VH is at least 90%, 91%, 92%, 93%, 94%, 95%, 96%, 97%, 98%, 99%, or 100% identical to the amino acid sequence of SEQ ID NO: 12 and the VL is at least 90%, 91%, 92%, 93%, 94%, 95%, 96%, 97%, 98%, 99%, or 100% identical to the amino acid sequence of SEQ ID NO: 18; or
ii) the VH is at least 90%, 91%, 92%, 93%, 94%, 95%, 96%, 97%, 98%, 99%, or 100% identical to the amino acid sequence of SEQ ID NO: 112 and the VL is at least 90%, 91%, 92%, 93%, 94%, 95%, 96%, 97%, 98%, 99%, or 100% identical to the amino acid sequence of SEQ ID NO: 118; or
iii) the VH is at least 90%, 91%, 92%, 93%, 94%, 95%, 96%, 97%, 98%, 99%, or 100% identical to the amino acid sequence of SEQ ID NO: 212 and the VL is at least 90%, 91%, 92%, 93%, 94%, 95%, 96%, 97%, 98%, 99%, or 100% identical to the amino acid sequence of SEQ ID NO: 218; or
iv) the VH is at least 90%, 91%, 92%, 93%, 94%, 95%, 96%, 97%, 98%, 99%, or 100% identical to the amino acid sequence of SEQ ID NO: 312 and the VL is at least 90%, 91%, 92%, 93%, 94%, 95%, 96%, 97%, 98%, 99%, or 100% identical to the amino acid sequence of SEQ ID NO: 318; or
v) the VH is at least 90%, 91%, 92%, 93%, 94%, 95%, 96%, 97%, 98%, 99%, or 100% identical to the amino acid sequence of SEQ ID NO: 412 and the VL is at least 90%, 91%, 92%, 93%, 94%, 95%, 96%, 97%, 98%, 99%, or 100% identical to the amino acid sequence of SEQ ID NO: 418; or
vi) the VH is at least 90%, 91%, 92%, 93%, 94%, 95%, 96%, 97%, 98%, 99%, or 100% identical to the amino acid sequence of SEQ ID NO: 512 and the VL is at least 90%, 91%, 92%, 93%, 94%, 95%, 96%, 97%, 98%, 99%, or 100% identical to the amino acid sequence of SEQ ID NO: 518; or
vii) the VH is at least 90%, 91%, 92%, 93%, 94%, 95%, 96%, 97%, 98%, 99%, or 100% identical to the amino acid sequence of SEQ ID NO: 612 and the VL is at least 90%, 91%, 92%, 93%, 94%, 95%, 96%, 97%, 98%, 99%, or 100% identical to the amino acid sequence of SEQ ID NO: 618; or
viii) the VH is at least 90%, 91%, 92%, 93%, 94%, 95%, 96%, 97%, 98%, 99%, or 100% identical to the amino acid sequence of SEQ ID NO: 712 and the VL is at least 90%, 91%, 92%, 93%, 94%, 95%, 96%, 97%, 98%, 99%, or 100% identical to the amino acid sequence of SEQ ID NO: 718; or
ix) the VH is at least 90%, 91%, 92%, 93%, 94%, 95%, 96%, 97%, 98%, 99%, or 100% identical to the amino acid sequence of SEQ ID NO: 812 and the VL is at least 90%, 91%, 92%, 93%, 94%, 95%, 96%, 97%, 98%, 99%, or 100% identical to the amino acid sequence of SEQ ID NO: 818; or
x) the VH is at least 90%, 91%, 92%, 93%, 94%, 95%, 96%, 97%, 98%, 99%, or 100% identical to the amino acid sequence of SEQ ID NO: 912 and the VL is at least 90%, 91%, 92%, 93%, 94%, 95%, 96%, 97%, 98%, 99%, or 100% identical to the amino acid sequence of SEQ ID NO: 918; or
xi) the VH is at least 90%, 91%, 92%, 93%, 94%, 95%, 96%, 97%, 98%, 99%, or 100% identical to the amino acid sequence of SEQ ID NO: 1012 and the VL is at least 90%, 91%, 92%, 93%, 94%, 95%, 96%, 97%, 98%, 99%, or 100% identical to the amino acid sequence of SEQ ID NO: 1018; or
xii) the VH is at least 90%, 91%, 92%, 93%, 94%, 95%, 96%, 97%, 98%, 99%, or 100% identical to the amino acid sequence of SEQ ID NO: 2012 and the VL is at least 90%, 91%, 92%, 93%, 94%, 95%, 96%, 97%, 98%, 99%, or 100% identical to the amino acid sequence of SEQ ID NO: 2018; or
xiii) the VH is at least 90%, 91%, 92%, 93%, 94%, 95%, 96%, 97%, 98%, 99%, or 100% identical to the amino acid sequence of SEQ ID NO: 3012 and the VL is at least 90%, 91%, 92%, 93%, 94%, 95%, 96%, 97%, 98%, 99%, or 100% identical to the amino acid sequence of SEQ ID NO: 3018; or
xiv) the VH is at least 90%, 91%, 92%, 93%, 94%, 95%, 96%, 97%, 98%, 99%, or 100% identical to the amino acid sequence of SEQ ID NO: 4012 and the VL is at least 90%, 91%, 92%, 93%, 94%, 95%, 96%, 97%, 98%, 99%, or 100% identical to the amino acid sequence of SEQ ID NO: 4018.

3. The isolated antibody of claim 1, wherein the antibody comprises a heavy chain variable region (VH) and a light chain variable region (VL), wherein:
i) the VH comprises the amino acid sequence of SEQ ID NO: 12 and the VL comprises the amino acid sequence of SEQ ID NO: 18; or
ii) the VH comprises the amino acid sequence of SEQ ID NO: 112 and the VL comprises the amino acid sequence of SEQ ID NO: 118; or
iii) the VH comprises the amino acid sequence of SEQ ID NO: 212 and the VL comprises the amino acid sequence of SEQ ID NO: 218; or
iv) the VH comprises the amino acid sequence of SEQ ID NO: 312 and the VL comprises the amino acid sequence of SEQ ID NO: 318; or
v) the VH comprises the amino acid sequence of SEQ ID NO: 412 and the VL comprises the amino acid sequence of SEQ ID NO: 418; or
vi) the VH comprises the amino acid sequence of SEQ ID NO: 512 and the VL comprises the amino acid sequence of SEQ ID NO: 518; or
vii) the VH comprises the amino acid sequence of SEQ ID NO: 612 and the VL comprises the amino acid sequence of SEQ ID NO: 618; or
viii) the VH comprises the amino acid sequence of SEQ ID NO: 712 and the VL comprises the amino acid sequence of SEQ ID NO: 718; or
ix) the VH comprises the amino acid sequence of SEQ ID NO: 812 and the VL comprises the amino acid sequence of SEQ ID NO: 818; or
x) the VH comprises the amino acid sequence of SEQ ID NO: 912 and the VL comprises the amino acid sequence of SEQ ID NO: 918; or
xi) the VH comprises the amino acid sequence of SEQ ID NO: 1012 and the VL comprises the amino acid sequence of SEQ ID NO: 1018; or
xii) the VH comprises the amino acid sequence of SEQ ID NO: 2012 and the VL comprises the amino acid sequence of SEQ ID NO: 2018; or
xiii) the VH comprises the amino acid sequence of SEQ ID NO: 3012 and the VL comprises the amino acid sequence of SEQ ID NO: 3018; or
xiv) the VH comprises the amino acid sequence of SEQ ID NO: 4012 and the VL comprises the amino acid sequence of SEQ ID NO: 4018.

4. The isolated antibody of claim 1, wherein the antibody is a monoclonal antibody.

5. The isolated antibody of claim 1, wherein the antibody is an antibody fragment.

6. The isolated antibody of claim 5, wherein the fragment is a Fab, Fab′, Fv, scFv or (Fab′)₂.

7. The isolated antibody of claim 1, wherein the antibody is a full-length antibody.

8. The isolated antibody of claim 1, wherein the Fc region of the antibody comprises IgG1, IgG2, IgG3, or IgG4.

9. The isolated antibody of claim 1, wherein the antibody comprises a human IgG1 heavy chain constant region.

10. The isolated antibody of claim 1, wherein the antibody comprises a human IgG4 heavy chain constant region.

11. The isolated antibody of claim 10, wherein the antibody comprises a mutant human IgG4 heavy chain constant region.

12. The isolated antibody of claim 11, wherein the mutant IgG4 heavy chain constant region comprises a mutation selected from a substitution at Ser228, a substitution at Leu235, a substitution at Asn297, or a combination thereof, numbering according to EU numbering.

13. The isolated antibody of claim 11, wherein the mutant IgG4 heavy chain constant region comprises an S228P substitution and an L235E substitution, numbering according to EU numbering.

14. The isolated antibody of claim 1, wherein the antibody comprises a human IgG1 heavy chain constant region, and wherein the antibody
i) increases NK cell degranulation; and/or
ii) increases activation of NK cells; and/or
iii) increases activation of intra-tumoral NK cells when presented in combination with an anti-TIGIT antibody; and/or
iv) inhibits tumor growth in vivo; and/or
v) prevents tumor engraftment upon re-challenge with tumor.

15. A humanized or fully human version of the antibody of claim 1.

16. A composition comprising the antibody of claim 1 and a pharmaceutically acceptable carrier.

17. The composition of claim 16 further comprising an antagonist of PD-1, PD-L1, CTLA-4, Lag-3 or TIM-3.

18. A method of enhancing, increasing and/or sustaining an anti-tumor immune response in a subject comprising administering the antibody of claim 1 to a subject having a tumor.

19. A method of enhancing CD8 T cell activation in a subject comprising administering the antibody of claim 1 to a subject in need of CD8 T cell activation.

20. A method of enhancing CD8 T cell interferon gamma production in a subject comprising administering the antibody of claim 1 to a subject in need of CD8 T cell interferon gamma production.

21. A method of enhancing NK cell activation in a subject comprising administering the antibody of claim 1 to a subject in need of NK cell activation.

22. A method of enhancing NK cell mediated cytotoxicity in a subject comprising administering the antibody of claim 1 to a subject in need of increasing NK cell mediated cytotoxicity.

23. A method of treating cancer in a subject comprising administering the antibody of claim 1 to a subject having cancer.

24. The method of claim 23, wherein the cancer is carcinoma, lymphoma, blastoma, sarcoma, or leukemia.

25. The method of claim 23, wherein the cancer is squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck).

26. A method of enhancing CD226 interactions with CD112 in a subject comprising administering the antibody of claim 1 to a subject.

27. The method of claim 18, wherein the method further comprises administering a second therapy.

28. The method of claim 27, wherein the second therapy is radiotherapy or surgery.

29. The method of claim 27, wherein the second therapy is administration of a chemotherapy, an opsonizing agent, or a regulatory T cell depleting agent.

30. The method of claim 27, wherein the second therapy is administration of an antagonist of PD-1, PD-L1, CTLA-4, Lag-3 or TIM-3.

31. The method of claim 27, wherein the second therapy is administration of an antagonist of TIGIT or CD96.

32. The method of claim 27, wherein the second therapy is administration of an antagonist of PVRL1, PVRL2, PVRL3, PVRL4, and CD155.

33. The method of claim 27, wherein the second therapy is administration of an antagonist of CD47.

34. The method of claim 27, wherein the second therapy is administration of an antagonist of CD39.

35. The method of claim 27, wherein the second therapy is administration of an antagonist of IL-27.

36. The method of claim 27, wherein the second therapy is administration of a STING agonist.

37. The method of claim 30, wherein the antagonist is an antibody.

38. A nucleic acid encoding the antibody of claim 1.

39. A host cell comprising the nucleic acid of claim 38.

40. A method of producing the antibody of claim 1 comprising culturing the host cell comprising a nucleic acid encoding the antibody of claim 1 under conditions wherein the antibody is expressed.

41. The method of claim 40, further comprising purifying the antibody.
